(19) United States
(12) Patent Application Publication
(10) Pub. No.: US 2022/0132846 A1
(43) Pub. Date: May 5, 2022

(54) 1-PYRIDYL-PYRAZOLYL AMIDE COMPOUNDS AND PREPARATION AND USES THEREOF

(71) Applicant: HUNAN RESEARCH INSTITUTE OF CHEMICAL INDUSTRY CO., LTD., Hunan (CN)

(72) Inventors: Aiping LIU, Hunan (CN); Yeguo REN, Hunan (CN); Weidong LIU, Hunan (CN); Chuyun LONG, Hunan (CN); Xiaoming OU, Hunan (CN); Chunge ZHOU, Hunan (CN); Lizhong LI, Hunan (CN); Li HU, Hunan (CN); Xingping LIU, Hunan (CN); Xuanqing KONG, Hunan (CN)

(73) Assignee: HUNAN RESEARCH INSTITUTE OF CHEMICAL INDUSTRY CO., LTD., Hunan (CN)

(21) Appl. No.: 17/437,030

(22) PCT Filed: Apr. 28, 2020
(86) PCT No.: PCT/CN2020/087491

§ 371 Date: Sep. 7, 2021

(30) Foreign Application Priority Data
Mar. 7, 2019 (CN) 201910172195.0

(51) Int. Cl. A01N 43/56; C07D 401/04; A01P 7/04

(52) U.S. Cl. CPC A01N 43/56; A01P 7/04; C07D 401/04

(57) ABSTRACT

This invention provides 1-pyridyl pyrazole amide compounds represented by Formula (I) and preparation and uses thereof, wherein each substituent is defined in the disclosure. The compounds of Formula (I) of this invention possess excellent insecticidal activities, especially very strong activities against pests such as aphids, armyworms and diamondback moths, etc., and can be used to control various pests. This invention also provides synthetic intermediates represented by Formula (II) for preparing compounds of Formula (I) wherein each substituent is defined in the specification.

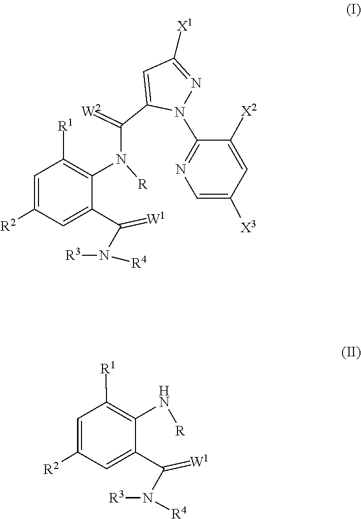

### BACKGROUND

### Field of the Disclosure

This invention belongs to the field of insecticide, specifically relates to 1-pyridyl-pyrazolyl amide compounds with insecticidal activity and preparation thereof, insecticidal compositions containing the compounds, and uses and methods for controlling pests with these compounds.

### Description of Related Art

1-pyridyl-pyrazolyl amides have broad-spectrum activity. 1-pyridyl-pyrazolyl amides, which are commercialized as insecticides, include chlorantraniliprole (D1), cyantraniliprole (D2), cyclaniliprole (D3), etc. As the leading insecticide for controlling lepidopteran pests, chlorantraniliprole (D1) has been on the market for only 10 years, but its sales have already reached $1.5 billion, ranking first in insecticide sales. As 1-pyridyl-pyrazolyl amide insecticides, chlorantraniliprole (D1), cyantraniliprole (D2), cyclaniliprole (D3), etc. have their own characteristics and advantages. Chlorantraniliprole (D1) has excellent controlling effect on lepidopteran pests such as Mythimna separata, Plutella xylostella, etc., but its controlling effect on homoptera pests such as Aphis fabae is not worth mentioning, and it does not have the potential and value as an insecticide for control homopteran pests such as Aphis fabae. The activities of cyantraniliprole (D2) and cyclaniliprole (D3) against homopteran pests such as Aphis fabae are significantly higher than those of chlorantraniliprole (D1), but their controlling effect on lepidopteran pests such as Mythimna separata, Plutella xylostella, etc. are no match for chlorantraniliprole (D1). As we all know, the leading insecticide for controlling homopteran pests such as Aphis fabae is neonicotine insecticides, and imidacloprid (D4) is its outstanding representative.

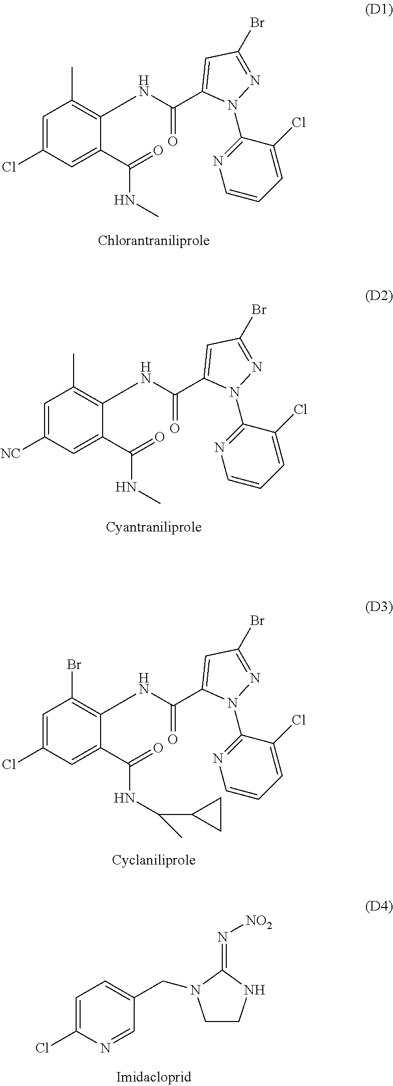 [SMILES: CNC(=O)c1cc(Cl)cc(C)c1NC(=O)c1cc(Br)nn1-c1ncccc1Cl ; CNC(=O)c1cc(C#N)cc(C)c1NC(=O)c1cc(Br)nn1-c1ncccc1Cl ; CC(NC(=O)c1cc(Cl)cc(Br)c1NC(=O)c1cc(Br)nn1-c1ncccc1Cl)C1CC1 ; O=[N+]([O-])/N=C1\NCCN1Cc1ccc(Cl)nc1]

To search for 1-pyridyl-pyrazolyl amides with more effective or broader-spectrum insecticidal activity, the inventors have designed and synthesized a series of novel 1-pyridyl-pyrazolyl amides with high-efficiency and broad-spectrum insecticidal activity by introducing specific substituents. Compared with chlorantraniliprole (D1), cyantraniliprole (D2) and cyclaniliprole (D3) etc., the compounds of this invention have the characteristics of broader-spectrum or more effective insecticidal activity.

### SUMMARY OF THE DISCLOSURE

This invention provides insecticidal 1-pyridyl-pyrazolyl amide compounds represented by Formula (I) and their isomers thereof:

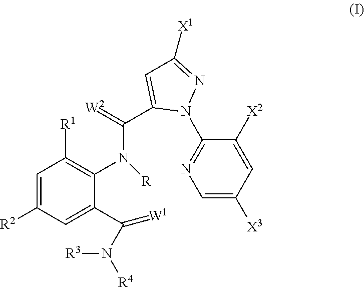

wherein:

I. R is C₁-C₁₂ alkyl, C₂-C₁₂ alkenyl, C₂-C₁₂ alkynyl, C₃-C₈ cycloalkyl or C₃-C₈ heterocyclic group;

II. R¹ is halogen, C₁-C₆ alkyl or C₁-C₆ alkoxy;

III. R² is halogen, cyano, C₁-C₆ alkyl, C₁-C₆ alkoxy or nitro;

IV. R³ is H, C₁-C₁₂ alkyl, C₂-C₁₂ alkenyl, C₂-C₁₂ alkynyl, C₃-C₈ cycloalkyl or C₃-C₈ heterocyclic group;

V. R⁴ is C₁-C₁₂ alkyl, C₂-C₁₂ alkenyl, C₂-C₁₂ alkynyl, C₃-C₈ cycloalkyl or C₃-C₈ heterocyclic group;

VI. X¹, X² and X³ may be identical or different, and they represent H, halogen, C₁-C₆ alkyl or C₁-C₆ alkoxy;

VII. W¹ and W² may be identical or different, and they represent oxygen or sulfur.

The hydrogen atoms in alkyl, alkenyl, alkynyl, cycloalkyl or heterocyclic group defined in I, II, III, IV, V and VI may be partially or entirely substituted by identical or different substituting groups selected from the following: halogen, cyano, hydroxyl, amino, mercapto, C₁-C₆ alkyl, C₁-C₆ alkoxy, C₁-C₆ alkylcarbonyl, C₁-C₆ alkoxycarbonyl, C₁-C₆ alkylthio, C₁-C₆ alkylamino, C₁-C₆ alkylsulfinyl, C₁-C₆ alkylsulfonyl or C₃-C₈ heterocyclic group.

In above definition of Formula (I), the terms used whether alone or in compound words, represent the following substituents:

halogen: represents fluorine, chlorine, bromine or iodine;

alkyl: represents straight-chain or branched-chain alkyl;

cycloalkyl: represents saturated or unsaturated cycloalkyl;

alkenyl: represents straight-chain or branched-chain alkenyl;

alkynyl: represents straight-chain or branched-chain alkynyl;

halo-: represents the hydrogen atoms may be partially or entirely substituted by halogen atoms;

heterocyclic group: represents saturated or unsaturated heterocyclic group containing one or more than one (identical or different) hetero-atoms selected from N, O and/or S, such as furanyl, thiophenyl, pyrrolyl, piperidinyl, piperazinyl, pyrazinyl, pyridazinyl, oxazolyl, thiazolyl, pyrazolyl, imidazolyl, tetrazolyl or triazolyl.

Preferred compounds of this invention are the compounds of Formula (I), wherein:

I. R is C₁-C₁₂ alkyl or C₃-C₅ cycloalkyl;

II. R¹ is halogen or CH₃;

III. R² is halogen, CF₃, NO₂ or CN;

IV. R³ is H, C₁-C₁₂ alkyl or C₃-C₅ cycloalkyl;

V. R⁴ is C₁-C₁₂ alkyl or C₃-C₅ cycloalkyl;

VI. X¹, X² and X³ may be identical or different, and they represent H, halogen or CF₃;

VII. W¹ and W² may be identical or different, and they represent oxygen or sulfur.

Further preferred compounds of this invention are the compounds of Formula (I), wherein:

I. R is C₁-C₄ alkyl or cyclopropyl;

II. R¹ is halogen or CH₃;

III. R² is halogen;

IV. R³ is H, C₁-C₆ alkyl or cyclopropyl;

V. R⁴ is C₁-C₆ alkyl or cyclopropyl;

VI. X¹, X² and X³ may be identical or different, and they represent H or halogen;

VII. W¹ and W² represent oxygen or sulfur.

Particularly preferred compounds of this invention are the compounds of Formula (I), wherein:

I. R is C₁-C₄ alkyl or cyclopropyl;

II. R¹ is chlorine or bromine;

III. R² is chlorine;

IV. R³ is H, C₁-C₄ alkyl or cyclopropyl;

V. R⁴ is C₁-C₄ alkyl or cyclopropyl;

VI. X¹ is chlorine or bromine, X² is chlorine, X³ is H or chlorine;

VII. W¹ and W² represent oxygen.

Specifically preferred compound of Formula (I) of this invention represents:
N-[2,4-dichloro-6-[(methylamino)carbonyl]phenyl]-N-methyl-1-(3-chloro-2-pyridinyl)-3-bromo-1H-pyrazole-5-carboxamide;
N-[2,4-dichloro-6-[(ethylamino)carbonyl]phenyl]-N-methyl-1-(3-chloro-2-pyridinyl)-3-bromo-1H-pyrazole-5-carboxamide;
N-[2,4-dichloro-6-[(propylamino)carbonyl]phenyl]-N-methyl-1-(3-chloro-2-pyridinyl)-3-bromo-1H-pyrazole-5-carboxamide;
N-[2,4-dichloro-6-[(isopropylamino)carbonyl]phenyl]-N-methyl-1-(3-chloro-2-pyridinyl)-3-bromo-1H-pyrazole-5-carboxamide;
N-[2,4-dichloro-6-[(cyclopropylamino)carbonyl]phenyl]-N-methyl-1-(3-chloro-2-pyridinyl)-3-bromo-1H-pyrazole-5-carboxamide;
N-[2,4-dichloro-6-[(butylamino)carbonyl]phenyl]-N-methyl-1-(3-chloro-2-pyridinyl)-3-bromo-1H-pyrazole-5-carboxamide;
N-[2,4-dichloro-6-[(tert-butylamino)carbonyl]phenyl]-N-methyl-1-(3-chloro-2-pyridinyl)-3-bromo-1H-pyrazole-5-carboxamide;
N-[2,4-dichloro-6-[(methylamino)carbonyl]phenyl]-N-ethyl-1-(3-chloro-2-pyridinyl)-3-bromo-1H-pyrazole-5-carboxamide;
N-[2,4-dichloro-6-[(ethylamino)carbonyl]phenyl]-N-ethyl-1-(3-chloro-2-pyridinyl)-3-bromo-1H-pyrazole-5-carboxamide;
N-[2,4-dichloro-6-[(dimethylamino)carbonyl]phenyl]-N-methyl-1-(3-chloro-2-pyridinyl)-3-bromo-1H-pyrazole-5-carboxamide;
N-[2,4-dichloro-6-[(methyl(ethyl)amino)carbonyl]phenyl]-N-methyl-1-(3-chloro-2-pyridinyl)-3-bromo-1H-pyrazole-5-carboxamide;
N-[2,4-dichloro-6-[(methyl(isopropyl)amino)carbonyl]phenyl]-N-methyl-1-(3-chloro-2-pyridinyl)-3-bromo-1H-pyrazole-5-carboxamide;
N-[2,4-dichloro-6-[(methyl(cyclopropyl)amino)carbonyl]phenyl]-N-methyl-1-(3-chloro-2-pyridinyl)-3-bromo-1H-pyrazole-5-carboxamide;
N-[2,4-dichloro-6-[(diethylamino)carbonyl]phenyl]-N-methyl-1-(3-chloro-2-pyridinyl)-3-bromo-1H-pyrazole-5-carboxamide;
N-[2,4-dichloro-6-[(dipropylamino)carbonyl]phenyl]-N-methyl-1-(3-chloro-2-pyridinyl)-3-bromo-1H-pyrazole-5-carboxamide;
N-[2,4-dichloro-6-[(dimethylamino)carbonyl]phenyl]-N-ethyl-1-(3-chloro-2-pyridinyl)-3-bromo-1H-pyrazole-5-carboxamide;
N-[2,4-dichloro-6-[(methyl(ethyl)amino)carbonyl]phenyl]-N-ethyl-1-(3-chloro-2-pyridinyl)-3-bromo-1H-pyrazole-5-carboxamide;
N-[2,4-dichloro-6-[(dimethylamino)carbonyl]phenyl]-N-propyl-1-(3-chloro-2-pyridinyl)-3-bromo-1H-pyrazole-5-carboxamide;
N-[2,4-dichloro-6-[(methyl(propyl)amino)carbonyl]phenyl]-N-propyl-1-(3-chloro-2-pyridinyl)-3-bromo-1H-pyrazole-5-carboxamide;
N-[2,4-dichloro-6-[(dimethylamino)thioxomethyl]phenyl]-N-methyl-1-(3-chloro-2-pyridinyl)-3-bromo-1H-pyrazole-5-carboxamide;
N-[2,4-dichloro-6-[(methyl(isopropyl)amino)thioxomethyl]phenyl]-N-methyl-1-(3-chloro-2-pyridinyl)-3-bromo-1H-pyrazole-5-carboxamide;
N-[2,4-dichloro-6-[(diethylamino)thioxomethyl]phenyl]-N-methyl-1-(3-chloro-2-pyridinyl)-3-bromo-1H-pyrazole-5-carboxamide;
N-[2,4-dichloro-6-[(diethylamino)thioxomethyl]phenyl]-N-methyl-1-(3-chloro-2-pyridinyl)-3-bromo-1H-pyrazole-5-carbothioamide;
N-[2-bromo-4-chloro-6-[(methylamino)carbonyl]phenyl]-N-methyl-1-(3-chloro-2-pyridinyl)-3-bromo-1H-pyrazole-5-carboxamide;
N-[2-bromo-4-chloro-6-[(ethylamino)carbonyl]phenyl]-N-methyl-1-(3-chloro-2-pyridinyl)-3-bromo-1H-pyrazole-5-carboxamide;
N-[2-bromo-4-chloro-6-[(propylamino)carbonyl]phenyl]-N-methyl-1-(3-chloro-2-pyridinyl)-3-bromo-1H-pyrazole-5-carboxamide;
N-[2-bromo-4-chloro-6-[(isopropylamino)carbonyl]phenyl]-N-methyl-1-(3-chloro-2-pyridinyl)-1H-pyrazole-5-carboxamide;
N-[2-bromo-4-chloro-6-[(isopropylamino)carbonyl]phenyl]-N-methyl-1-(3-chloro-2-pyridinyl)-3-bromo-1H-pyrazole-5-carboxamide;
N-[2-bromo-4-chloro-6-[(cyclopropylamino)carbonyl]phenyl]-N-methyl-1-(3-chloro-2-pyridinyl)-3-bromo-1H-pyrazole-5-carboxamide;
N-[2-bromo-4-chloro-6-[(butylamino)carbonyl]phenyl]-N-methyl-1-(3-chloro-2-pyridinyl)-1H-pyrazole-5-carboxamide;
N-[2-bromo-4-chloro-6-[(butylamino)carbonyl]phenyl]-N-methyl-1-(3-chloro-2-pyridinyl)-3-bromo-1H-pyrazole-5-carboxamide;
N-[2-bromo-4-chloro-6-[(dimethylamino)carbonyl]phenyl]-N-methyl-1-(3-chloro-2-pyridinyl)-3-bromo-1H-pyrazole-5-carboxamide;
N-[2-bromo-4-chloro-6-[(methyl(ethyl)amino)carbonyl]phenyl]-N-methyl-1-(3-chloro-2-pyridinyl)-3-bromo-1H-pyrazole-5-carboxamide;
N-[2-bromo-4-chloro-6-[(methyl(propyl)amino)carbonyl]phenyl]-N-methyl-1-(3-chloro-2-pyridinyl)-3-bromo-1H-pyrazole-5-carboxamide;
N-[2-bromo-4-chloro-6-[(methyl(isopropyl)amino)carbonyl]phenyl]-N-methyl-1-(3-chloro-2-pyridinyl)-3-bromo-1H-pyrazole-5-carboxamide;
N-[2-bromo-4-chloro-6-[(methyl(cyclopropyl)amino)carbonyl]phenyl]-N-methyl-1-(3-chloro-2-pyridinyl)-3-bromo-1H-pyrazole-5-carboxamide;
N-[2-bromo-4-chloro-6-[(methyl(tert-butyl)amino)carbonyl]phenyl]-N-methyl-1-(3-chloro-2-pyridinyl)-3-bromo-1H-pyrazole-5-carboxamide;
N-[2-bromo-4-chloro-6-[(diethylamino)carbonyl]phenyl]-N-methyl-1-(3-chloro-2-pyridinyl)-3-bromo-1H-pyrazole-5-carboxamide;
N-[2-bromo-4-chloro-6-[(dimethylamino)carbonyl]phenyl]-N-ethyl-1-(3-chloro-2-pyridinyl)-3-bromo-1H-pyrazole-5-carboxamide;
N-[2-bromo-4-chloro-6-[(methyl(ethyl)amino)carbonyl]phenyl]-N-ethyl-1-(3-chloro-2-pyridinyl)-3-bromo-1H-pyrazole-5-carboxamide;
N-[2-bromo-4-chloro-6-[(diethylamino)carbonyl]phenyl]-N-ethyl-1-(3-chloro-2-pyridinyl)-3-bromo-1H-pyrazole-5-carboxamide;
N-[2-bromo-4-chloro-6-[(dimethylamino)carbonyl]phenyl]-N-propyl-1-(3-chloro-2-pyridinyl)-3-bromo-1H-pyrazole-5-carboxamide;
N-[2-methyl-4-chloro-6-[(methylamino)carbonyl]phenyl]-N-methyl-1-(3-chloro-2-pyridinyl)-3-bromo-1H-pyrazole-5-carboxamide;
N-[2-methyl-4-chloro-6-[(ethylamino)carbonyl]phenyl]-N-methyl-1-(3-chloro-2-pyridinyl)-3-bromo-1H-pyrazole-5-carboxamide;
N-[2-methyl-4-chloro-6-[(dimethylamino)carbonyl]phenyl]-N-methyl-1-(3-chloro-2-pyridinyl)-3-bromo-1H-pyrazole-5-carboxamide;
N-[2-methyl-4-chloro-6-[(methyl(ethyl)amino)carbonyl]phenyl]-N-methyl-1-(3-chloro-2-pyridinyl)-3-bromo-1H-pyrazole-5-carboxamide;
N-[2-methyl-4-chloro-6-[(methyl(propyl)amino)carbonyl]phenyl]-N-methyl-1-(3-chloro-2-pyridinyl)-3-bromo-1H-pyrazole-5-carboxamide;
N-[2-methyl-4-chloro-6-[(dimethylamino)thioxomethyl]phenyl]-N-methyl-1-(3-chloro-2-pyridinyl)-3-bromo-1H-pyrazole-5-carboxamide;
N-[2-methyl-4-bromo-6-[(diethylamino)carbonyl]phenyl]-N-methyl-1-(3-chloro-2-pyridinyl)-3-bromo-1H-pyrazole-5-carboxamide;
N-[2-methyl-4-bromo-6-[(diethylamino)carbonyl]phenyl]-N-ethyl-1-(3-chloro-2-pyridinyl)-3-bromo-1H-pyrazole-5-carboxamide.

The compounds of Formula (II) are important synthetic intermediates for preparing the compounds of Formula (I) of this invention, and their structures and physicochemical properties have not been reported. Therefore, this invention also relates to the synthetic intermediates represented by Formula (II):

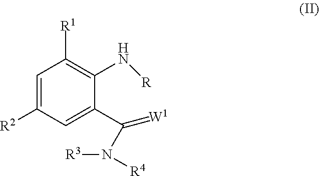

wherein:

R is C₁-C₁₂ alkyl, C₂-C₁₂ alkenyl, C₂-C₁₂ alkynyl, C₃-C₈ cycloalkyl or C₃-C₈ heterocyclic group;

R¹ is halogen, C₁-C₆ alkyl or C₁-C₆ alkoxy;

R² is halogen, cyano, C₁-C₆ alkyl, C₁-C₆ alkoxy or nitro;

R³ is H, C₁-C₁₂ alkyl, C₂-C₁₂ alkenyl, C₂-C₁₂ alkynyl, C₃-C₈ cycloalkyl or C₃-C₈ heterocyclic group;

R⁴ is C₁-C₁₂ alkyl, C₂-C₁₂ alkenyl, C₂-C₁₂ alkynyl, C₃-C₈ cycloalkyl or C₃-C₈ heterocyclic group;

W¹ represents oxygen or sulfur;

The hydrogen atoms in alkyl, alkenyl, alkynyl, cycloalkyl or heterocyclic group defined in R, R¹, R², R³ and R⁴ may be partially or entirely substituted by identical or different substituting groups selected from the following: halogen, cyano, hydroxyl, amino, mercapto, C₁-C₆ alkyl, C₁-C₆ alkoxy, C₁-C₆ alkylcarbonyl, C₁-C₆ alkoxycarbonyl, C₁-C₆ alkylthio, C₁-C₆ alkylamino, C₁-C₆ alkylsulfinyl, C₁-C₆ alkylsulfonyl or C₃-C₈ heterocyclic group.

In above definition of Formula (II), the terms used whether alone or in compound words, represent the following substituents:

halogen: represents fluorine, chlorine, bromine or iodine;

alkyl: represents straight-chain or branched-chain alkyl;

cycloalkyl: represents saturated or unsaturated cycloalkyl;

alkenyl: represents straight-chain or branched-chain alkenyl;

alkynyl: represents straight-chain or branched-chain alkynyl;

halo-: represents the hydrogen atoms may be partially or entirely substituted by halogen atoms;

heterocyclic group: represents saturated or unsaturated heterocyclic group containing one or more than one (identical or different) hetero-atoms selected from N, O and/or S, such as furanyl, thiophenyl, pyrrolyl, piperidinyl, piperazinyl, pyrazinyl, pyridazinyl, oxazolyl, thiazolyl, pyrazolyl, imidazolyl, tetrazolyl or triazolyl.

The preferred compound of Formula (II) of this invention represents:
N-methyl-3,5-dichloro-2-(methylamino)benzamide;
N-ethyl-3,5-dichloro-2-(methylamino)benzamide;
N-propyl-3,5-dichloro-2-(methylamino)benzamide;
N-isopropyl-3,5-dichloro-2-(methylamino)benzamide;
N-cyclopropyl-3,5-dichloro-2-(methylamino)benzamide;
N-butyl-3,5-dichloro-2-(methylamino)benzamide;
N-methyl-3,5-dichloro-2-(ethylamino)benzamide;
N-ethyl-3,5-dichloro-2-(ethylamino)benzamide;
N,N-dimethyl-3,5-dichloro-2-(methylamino)benzamide;
N-methyl-N-ethyl-3,5-dichloro-2-(methylamino)benzamide;
N-methyl-N-isopropyl-3,5-dichloro-2-(methylamino)benzamide;
N-methyl-N-cyclopropyl-3,5-dichloro-2-(methylamino)benzamide;
N,N-diethyl-3,5-dichloro-2-(methylamino)benzamide;
N,N-dipropyl-3,5-dichloro-2-(methylamino)benzamide;
N,N-dimethyl-3,5-dichloro-2-(ethylamino)benzamide;
N-methyl-N-ethyl-3,5-dichloro-2-(ethylamino)benzamide;
N,N-dimethyl-3,5-dichloro-2-(propylamino)benzamide;
N-methyl-N-propyl-3,5-dichloro-2-(propylamino)benzamide;
N,N-dimethyl-3,5-dichloro-2-(methylamino)benzothioamide;
N-methyl-N-isopropyl-3,5-dichloro-2-(methylamino)benzothioamide;
N,N-diethyl-3,5-dichloro-2-(methylamino)benzothioamide;
N-methyl-3-bromo-5-chloro-2-(methylamino)benzamide;
N-ethyl-3-bromo-5-chloro-2-(methylamino)benzamide;
N-propyl-3-bromo-5-chloro-2-(methylamino)benzamide;
N-isopropyl-3-bromo-5-chloro-2-(methylamino)benzamide;
N-cyclopropyl-3-bromo-5-chloro-2-(methylamino)benzamide;
N-butyl-3-bromo-5-chloro-2-(methylamino)benzamide;
N,N-dimethyl-3-bromo-5-chloro-2-(methylamino)benzamide;
N-methyl-N-ethyl-3-bromo-5-chloro-2-(methylamino)benzamide;
N-methyl-N-propyl-3-bromo-5-chloro-2-(methylamino)benzamide;
N-methyl-N-isopropyl-3-bromo-5-chloro-2-(methylamino)benzamide;
N-methyl-N-cyclopropyl-3-bromo-5-chloro-2-(methylamino)benzamide;
N-methyl-N-tert-butyl-3-bromo-5-chloro-2-(methylamino)benzamide;
N,N-diethyl-3-bromo-5-chloro-2-(methylamino)benzamide;
N,N-dimethyl-3-bromo-5-chloro-2-(ethylamino)benzamide;
N-methyl-N-ethyl-3-bromo-5-chloro-2-(ethylamino)benzamide;
N,N-diethyl-3-bromo-5-chloro-2-(ethylamino)benzamide;
N,N-dimethyl-3-bromo-5-chloro-2-(propylamino)benzamide;
N-methyl-3-methyl-5-chloro-2-(methylamino)benzamide;
N-ethyl-3-methyl-5-chloro-2-(methylamino)benzamide;
N,N-dimethyl-3-methyl-5-chloro-2-(methylamino)benzamide;
N-methyl-N-ethyl-3-methyl-5-chloro-2-(methylamino)benzamide;
N,N-dimethyl-3-methyl-5-chloro-2-(methylamino)benzothioamide;
N,N-diethyl-3-methyl-5-bromo-2-(methylamino)benzamide;
N,N-diethyl-3-methyl-5-bromo-2-(ethylamino)benzamide;
N-methyl-N-propyl-3-methyl-5-chloro-2-(methylamino)benzamide.

Compounds of this invention can exist as one or more isomers. Isomers of compounds of Formula (I) or compounds of Formula (II) include enantiomers, diastereomers, geometric isomers, rotational isomers and tautomers. The compounds of Formula (I) or the compounds of Formula (II) of this invention can exist as geometric isomers due to different substituents on carbon carbon double bonds, and the geometric isomer is represented by Z or E, this invention includes Z-isomer or E-isomer and their mixture in any proportion. The compounds of Formula (I) or the compounds of Formula (II) of this invention can exist as stereoisomers because of four different substituents on the same carbon atom or three different substituents on the same nitrogen atom, and the stereoisomer is represented by R or S, this invention includes R-isomer or S-isomer and their mixture in any proportion. The compounds of Formula (I) or the compounds of Formula (II) of this invention can exist as rotational isomers due to the partial double-bond feature of the amide bond, and the rotational isomer is represented by I or I′, this invention includes I-isomer or I′-isomer and their mixture in any proportion. The compounds of Formula (I) or the compounds of Formula (II) of this invention can exist as keto-enol tautomers due to the tautomerism of carbonyl bond, and the keto-enol tautomer is represented by I or I″, this invention includes I-isomer or I″ isomers and their mixture in any proportion.

This invention also relates to a composition for controlling pests comprising a biologically effective amount of a compound of Formula (I) and at least one additional component selected from the group consisting of surfactants, solid diluents and liquid diluents.

This invention also relates to a composition for controlling pests comprising a biologically effective amount of a compound of Formula (I) and an effective amount of at least one additional biologically active compound or formulation.

This invention also relates to a method for controlling pests comprising contacting the pest or its environment with a biologically effective amount of a compound of Formula (I). This invention also relates to such a method wherein the pest or its environment is contacted with a biologically effective amount of a compound of Formula (I) or a composition comprising a compound of Formula (I) and a biologically effective amount of at least one additional compound or formulation.

The compounds of Formula (I) of this invention have broad-spectrum activity: these compounds can be used to control pests. Moreover, some compounds have very high biological activity against some pests, so that very good efficacy can be obtained at very low concentration.

The preferred compositions of this invention are those which comprise the above preferred compounds. The preferred methods are those involving the above preferred compounds.

This invention can be further explained with the compounds of Formula (I) listed in Table 1-2 and the compounds of Formula (II) listed in Table 3-4, but this invention is not limited to the compounds in Table 1-2 and Table 3-4. The melting points given in this invention are not corrected. When the compounds of Formula (I) and Formula (II) synthesized of this invention are viscous liquid or viscous solid, some viscous liquid or viscous solid will be solidified after being placed. Compounds in Table 1 were identified by LC-MS (APCI, Pos) (Agilent 1260/6120 series LC/MSD) and the molecular ion M⁺+1 can be observed. Compounds in Table 3 were identified by LC-MS (APCI, Pos) (Agilent 1260/6120 series LC/MSD) and GC-MS (EI, 70 Ev m/z) (Agilent 7890-5975C Series), the molecular ion M⁺+1 in LC-MS or M⁺ in GC-MS can be observed; ¹H NMR (Varian INOVA-300 spectrometer or Varian INOVA-500 spectrometer) of the compounds in Table 1 and Table 3 use tetramethylsilane (TMS) as the internal standard and deuterated chloroform (CDCl₃) or deuterated dimethyl sulfoxide (DMSO) as the solvent.

For the compounds of Formula (I) with W¹=O and X¹≠H, although their purity of HPLC analysis are very high and their LC-MS are reasonable, there are mostly two groups of peaks in their ¹H NMR measured at 25° C. There are still two groups of peaks in their variable-temperature ¹H NMR. The peak width of hydrogen on pyrazole ring decreases with the increase of temperature. For simplicity, except compounds 1, 2, 11, 12 and 15, only one group of peaks in ¹H NMR are listed in Table 2. The reason for two groups of peaks in ¹H NMR probably be rotational isomerism of the compounds of Formula (I) because of the partial double-bond feature of the amide bond. Two groups of peaks probably correspond to the compounds of Formula (I) and its rotational isomer of formula (P′) respectively. For compound 11, etc., their single crystals were cultured (dichloromethane/ether) and their structure were confirmed by single-crystal X-ray diffraction structural studies. The structures of the compounds of Formula (I) can be confirmed by combining their HPLC analysis results, MS, NMR and single-crystal X-ray diffraction structural studies. The structures of the compounds in Table 3 were confirmed by MS, and most of them were further confirmed by NMR. For simplicity, Table 4 only lists out the NMR and physical properties of some compounds of Formula (II).

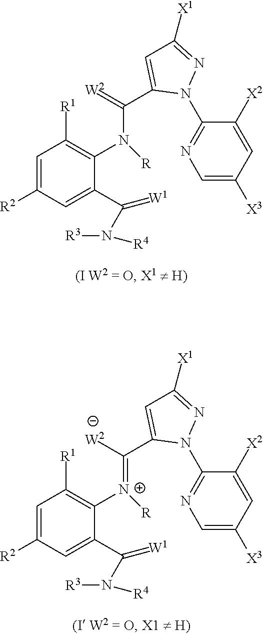

TABLE 1

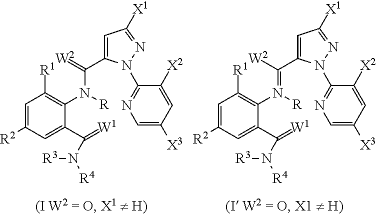

| No | R | R¹ | R² | R³ | R⁴ | W¹ | W² | X¹ | X² | X³ |
|---|---|---|---|---|---|---|---|---|---|---|
| 1 | CH₃ | Cl | Cl | H | CH₃ | O | O | Br | Cl | H |
| 2 | CH₃ | Cl | Cl | H | CH₂CH₃ | O | O | Br | Cl | H |
| 3 | CH₃ | Cl | Cl | H | CH₂CH₂CH₃ | O | O | Br | Cl | H |
| 4 | CH₃ | Cl | Cl | H | CH(CH₃)₂ | O | O | Cl | Cl | H |
| 5 | CH₃ | Cl | Cl | H | CH(CH₃)₂ | O | O | Br | Cl | H |
| 6 | CH₃ | Cl | Cl | H | 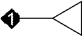 | O | O | Br | Cl | H |
| 7 | CH₃ | Cl | Cl | H | CH₂CH₂CH₂CH₃ | O | O | Br | Cl | H |
| 8 | CH₃ | Cl | Cl | H | C(CH₃)₃ | O | O | Br | Cl | H |
| 9 | CH₂CH₃ | Cl | Cl | H | CH₃ | O | O | Br | Cl | H |
| 10 | CH₂CH₃ | Cl | Cl | H | CH₂CH₃ | O | O | Br | Cl | H |
| 11 | CH₃ | Cl | Cl | CH₃ | CH₃ | O | O | Br | Cl | H |
| 12 | CH₃ | Cl | Cl | CH₃ | CH₂CH₃ | O | O | Br | Cl | H |
| 13 | CH₃ | Cl | Cl | CH₃ | CH(CH₃)₂ | O | O | Br | Cl | H |
| 14 | CH₃ | Cl | Cl | CH₃ | 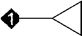 | O | O | Br | Cl | H |
| 15 | CH₃ | Cl | Cl | CH₂CH₃ | CH₂CH₃ | O | O | Br | Cl | H |
| 16 | CH₃ | Cl | Cl | CH₂CH₂CH₃ | CH₂CH₂CH₃ | O | O | Br | Cl | H |
| 17 | CH₂CH₃ | Cl | Cl | CH₃ | CH₃ | O | O | Br | Cl | H |
| 18 | CH₂CH₃ | Cl | Cl | CH₃ | CH₂CH₃ | O | O | Br | Cl | H |
| 19 | CH₂CH₂CH₃ | Cl | Cl | CH₃ | CH₃ | O | O | Br | Cl | H |
| 20 | CH₂CH₂CH₃ | Cl | Cl | CH₃ | CH₂CH₂CH₃ | O | O | Br | Cl | H |
| 21 | CH₃ | Cl | Cl | CH₃ | CH₃ | S | O | Br | Cl | H |
| 22 | CH₃ | Cl | Cl | CH₃ | CH(CH₃)₂ | S | O | Br | Cl | H |
| 23 | CH₃ | Cl | Cl | CH₂CH₃ | CH₂CH₃ | S | O | Br | Cl | H |
| 24 | CH₃ | Cl | Cl | CH₂CH₃ | CH₂CH₃ | S | S | Br | Cl | H |
| 25 | CH₃ | Br | Cl | H | CH₃ | O | O | Br | Cl | H |
| 26 | CH₃ | Br | Cl | H | CH₂CH₃ | O | O | Br | Cl | H |
| 27 | CH₃ | Br | Cl | H | CH₂CH₂CH₃ | O | O | Br | Cl | H |
| 28 | CH₃ | Br | Cl | H | CH(CH₃)₂ | O | O | H | Cl | H |
| 29 | CH₃ | Br | Cl | H | 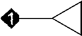 | O | O | Br | Cl | H |
| 30 | CH₃ | Br | Cl | H | CH₂CH₂CH₂CH₃ | O | O | H | Cl | H |
| 31 | CH₃ | Br | Cl | CH₃ | CH₃ | O | O | Br | Cl | H |
| 32 | CH₃ | Br | Cl | CH₃ | CH₂CH₃ | O | O | Br | Cl | H |
| 33 | CH₃ | Br | Cl | CH₃ | CH₂CH₂CH₃ | O | O | Br | Cl | H |
| 34 | CH₃ | Br | Cl | CH₃ | CH(CH₃)₂ | O | O | Br | Cl | H |
| 35 | CH₃ | Br | Cl | CH₃ | 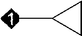 | O | O | Br | Cl | H |
| 36 | CH₃ | Br | Cl | CH₃ | C(CH₃)₃ | O | O | Br | Cl | H |
| 37 | CH₃ | Br | Cl | CH₂CH₃ | CH₂CH₃ | O | O | Br | Cl | H |
| 38 | CH₂CH₃ | Br | Cl | CH₃ | CH₃ | O | O | Br | Cl | H |
| 39 | CH₂CH₃ | Br | Cl | CH₃ | CH₂CH₃ | O | O | Br | Cl | H |
| 40 | CH₂CH₃ | Br | Cl | CH₂CH₃ | CH₂CH₃ | O | O | Br | Cl | H |
| 41 | CH₂CH₂CH₃ | Br | Cl | CH₃ | CH₃ | O | O | Br | Cl | H |
| 42 | CH₃ | CH₃ | Cl | H | CH₃ | O | O | Br | Cl | H |
| 43 | CH₃ | CH₃ | Cl | H | CH₂CH₃ | O | O | Br | Cl | H |
| 44 | CH₃ | CH₃ | Cl | CH₃ | CH₃ | O | O | Br | Cl | H |
| 45 | CH₃ | CH₃ | Cl | CH₃ | CH₂CH₃ | O | O | Br | Cl | H |
| 46 | CH₃ | CH₃ | Cl | CH₃ | CH₂CH₂CH₃ | O | O | Br | Cl | H |
| 47 | CH₃ | CH₃ | Cl | CH₃ | CH₃ | S | O | Br | Cl | H |
| 48 | CH₃ | CH₃ | Br | CH₂CH₃ | CH₂CH₃ | O | O | Br | Cl | H |
| 49 | CH₂CH₃ | CH₃ | Br | CH₂CH₃ | CH₂CH₃ | O | O | Br | Cl | H |
| 50 | CH₂CH₃ | CH₃ | Br | CH₂CH₃ | CH₂CH₃ | O | O | Cl | Cl | H |

TABLE 2

| No. | Physical Properties and ¹H NMR (ppm) |
|---|---|
| 1 | N-[2,4-dichloro-6-[(methylamino)carbonyl]phenyl]-N-methyl-1-(3-chloro-2- |
| | pyridinyl)-3-bromo-1H-pyrazole-5-carboxamide. |
| | white solid, m.p. 198.1-199.0° C. |
| | ¹H NMR (CDCl₃) δ: a first group of peaks: 2.865 (d, J = 4.5 Hz, 3H, CH₃), 3.499 (s, |
| | 3H, CH₃), 6.110 (br, 1H, NH), 6.843 (s, 1H, Pyrazole H), 7.345 (dd, J = 8.1 Hz, 4.5 |
| | Hz, 1H, Py H), 7.372 (d, J = 1.8 Hz, 1H, Ph H), 7.527 (d, J = 1.8 Hz, 1H, Ph H), 7.883 |
| | (d, J = 7.8 Hz, 1H, Py H), 8.424 (d, J = 4.8 Hz, 1H, Py H). a second group of peaks: δ: |
| | 2.962 (d, J = 4.5 Hz, 3H, CH₃), 3.233 (s, 3H, CH₃), 5.580 (s, 1H, Pyrazole H), 5.858 |
| | (br, 1H, NH), 7.387 (dd, J = 8.1 Hz, 4.5 Hz, 1H, Py H), 7.521 (d, J = 1.8 Hz, 1H, Ph H), |
| | 7.663 (d, J = 2.1 Hz, 1H, Ph H), 7.906 (d, J = 7.8 Hz 1H, Py H), 8.413 (d, J = 4.8 Hz, |
| | 1H, Py H). |
| | LC-MS (Pos M⁺) m/z) calc: 516, found: 516. |
| 2 | N-[2,4-dichloro-6-[(ethylamino)carbonyl]phenyl]-N-methyl-1-(3-chloro-2- |
| | pyridinyl)-3-bromo-1H-pyrazole-5-carboxamide. |
| | white solid, m.p. 230.8-232.1° C. |
| | ¹H NMR (CDCl₃) δ: a first group of peaks: 1.092 (t, J = 7.2 Hz, 3H, CH₃), 3.275-3.386 |
| | (m, 2H, CH₂), 3.500 (s, 3H, CH₃), 6.058 (br, 1H, NH), 6.838 (s, 1H, Pyrazole H), |
| | 7.336 (dd, J = 8.1 Hz, 4.5 Hz, 1H, Py H), 7.362 (d, J = 1.8 Hz, 1H, Ph H), 7.536 (d, |
| | J = 1.8 Hz, 1H, Ph H), 7.869 (d, J = 8.1 Hz 1H, Py H), 8.416 (d, J = 4.5 Hz, 1H, Py H). |
| | a second group of peaks: δ: 1.236 (t, J = 7.2 Hz, 3H, CH₃), 3.228 (s, 3H, CH₃), 3.275- |
| | 3.386 (m, 2H, CH₂), 5.586 (s, 1H, Pyrazole H), 5.821 (br, 1H, NH), 7.513 (s, 1H, Ph |
| | H), 7.386 (dd, J = 8.1 Hz, 5.1 Hz, 1H, Py H), 7.654 (s, 1H, Ph H), 7.896 (d, J = 8.1 Hz |
| | 1H, Py H), 8.404 (d, J = 4.5 Hz, 1H, Py H). |
| | LC-MS (Pos M⁺) m/z) calc: 530, found: 530. |
| 3 | N-[2,4-dichloro-6-[(propylamino)carbonyl]phenyl]-N-methyl-1-(3-chloro-2- |
| | pyridinyl)-3-bromo-1H-pyrazole-5-carboxamide. |
| | white solid, m.p. 175.6-178.1° C. |
| | ¹H NMR (CDCl₃) δ: 0.866 (t, J = 7.2 Hz, 3H, CH₃), 1.432-1.554 (m, 2H, CH₂), 3.132- |
| | 3.348 (m, 2H, CH₂), 3.501 (s, 3H, CH₃), 6.031 (br, 1H, NH), 6.836 (s, 1H, Pyrazole |
| | H), 7.336 (dd, J = 8.1 Hz, 4.5 Hz, 1H, Py H), 7.366 (d, J = 1.8 Hz, 1H, Ph H), 7.533 (d, |
| | J = 1.8 Hz, 1H, Ph H), 7.919 (d, J = 7.5 Hz 1H, Py H), 8.417 (s, 1H, Py H). |
| | LC-MS (Pos M⁺) m/z) calc: 544, found: 544. |
| 5 | N-[2,4-dichloro-6-[(isopropylamino)carbonyl]phenyl]-N-methyl-1-(3-chloro-2- |
| | pyridinyl)-3-bromo-1H-pyrazole-5-carboxamide. |
| | white solid, m.p. 201.3-202.7° C. |
| | ¹H NMR (CDCl₃) δ: 1.059 (d, J = 6.6 Hz, 3H, CH₃), 1.181 (d, J = 6.6 Hz, 3H, CH₃), |
| | 3.505 (s, 3H, CH₃), 4.123-4.188 (m, 1H, CH), 5.796 (d, J = 7.5 Hz, 1H, NH), 6.846 (s, |
| | 1H, Pyrazole H), 7.322 (d, J = 2.1 Hz, 1H, Ph H), 7.328 (dd, J = 8.1 Hz, 4.5 Hz, 1H, Py |
| | H), 7.500 (d, J = 2.1 Hz, 1H, Ph H), 7.856 (d, J = 7.8 Hz 1H, Py H), 8.425 (s, 1H, Py H). |
| | LC-MS (Pos M⁺) m/z) calc: 544, found: 544. |
| 6 | N-[2,4-dichloro-6-[(cyclopropylamino)carbonyl]phenyl]-N-methyl-1-(3-chloro-2- |
| | pyridinyl)-3-bromo-1H-pyrazole-5-carboxamide. |
| | white solid, m.p. 178.3-180.5° C. |
| | ¹H NMR (CDCl₃) δ: 0.421-0.851 (m, 4H, 2CH₂), 2.712 (br, 1H, CH), 3.497 (s, 3H, |
| | CH₃), 6.251 (br, 1H, NH), 6.873 (s, 1H, Pyrazole H), 7.269 (s, 1H, Ph H), 7.333 (dd, |
| | J = 8.1 Hz, 4.8 Hz, 1H, Py H), 7.495 (s, 1H, Ph H), 7.871 (d, J = 6.9 Hz 1H, Py H), |
| | 8.438 (s, 1H, Py H). |
| | LC-MS (Pos M⁺) m/z) calc: 542, found: 542. |
| 7 | N-[2,4-dichloro-6-[(butylamino)carbonyl]phenyl]-N-methyl-1-(3-chloro-2- |
| | pyridinyl)-3-bromo-1H-pyrazole-5-carboxamide. |
| | light yellow solid, m.p. 73.8-75.6° C. |
| | ¹H NMR (CDCl₃) δ: 0.864 (d, J = 7.2 Hz, 3H, CH₃), 1.255-1.380 (m, 4H, 2CH₂), |
| | 3.243-3.460 (m, 2H, CH₂), 3.503 (s, 3H, CH₃), 6.029 (br, 1H, NH), 6.836 (s, 1H, |
| | Pyrazole H), 7.332 (dd, J = 8.1 Hz, 4.5 Hz, 1H, Py H), 7.344 (d, J = 1.8 Hz, 1H, Ph H), |
| | 7.587 (d, J = 1.8 Hz, 1H, Ph H), 7.864 (d, J = 8.1 Hz, 1H, Py H), 8.416 (s, 1H, Py H). |
| | LC-MS (Pos M⁺) m/z) calc: 558, found: 558. |
| 9 | N-[2,4-dichloro-6-[(methylamino)carbonyl]phenyl]-N-ethyl-1-(3-chloro-2- |
| | pyridinyl)-3-bromo-1H-pyrazole-5-carboxamide. |
| | light yellow solid, m.p. >250° C. |
| | ¹H NMR (CDCl₃) δ: a first group of peaks: 1.100 (t, J = 7.2 Hz, 3H, CH₃), 3.283- |
| | 3.420 (m, 2H, CH₂), 3.506 (s, 3H, CH₃), 6.024 (br, 1H, NH), 6.843 (s, 1H, pyrazole |
| | H), 7.342 (dd, J = 8.1 Hz, 4.5 Hz, 1H, Py H), 7.394 (s, 1H, Ph H), 7.520(s, 1H, Ph H), |
| | 7.873 (d, J = 8.1 Hz, 1H, Py H), 8.409 (s, 1H, Py H). a second group of peaks: 1.250 |
| | (t, J = 7.2 Hz, 3H, CH₃), 3.283-3.420 (m, 2H, CH₂), 3.234 (s, 3H, CH₃), 5.583 (s, 1H, |
| | pyrazole H), 5.804 (br, 1H, NH), 7.358 (dd, J = 8.1 Hz, 4.5 Hz, 1H, Py H), 7.526 (s, |
| | 1H, Ph H), 7.659 (s, 1H, Ph H), 7.901 (d, J = 8.1 Hz, 1H, Py H), 8.422 (s, 1H, Py H). |
| | LC-MS (Pos M⁺) m/z) calc: 558, found: 530. |
| 10 | N-[2,4-dichloro-6-[(ethylamino)carbonyl]phenyl]-N-ethyl-1-(3-chloro-2- |
| | pyridinyl)-3-bromo-1H-pyrazole-5-carboxamide. |
| | white solid, m.p. 182.7-184.3° C. |
| | ¹H NMR (CDCl₃) δ: 1.090 (t, J = 7.2 Hz, 3H, CH₃), 1.196 (t, J = 7.2 Hz, 3H, CH₃), |
| | 3.489-3.448 (m, 2H, CH₂), 3.763-3.810 (m, 2H, CH₂), 6.313 (br, 1H, NH), 6.794 (s, |
| | 1H, pyrazole H), 7.357 (dd, J = 8.1 Hz, 4.5 Hz, 1H, Py H), 7.468(s, 1H, Ph H), |
| | 7.514(s, 1H, Ph H), 7.890 (d, J = 7.8 Hz, 1H, Py H), 8.400 (d, J = 4.5 Hz, 1H, Py H). |
| | LC-MS (Pos M⁺) m/z) calc: 544, found: 544. |
| 11 | N-[2,4-dichloro-6-[(dimethylamino)carbonyl]phenyl]-N-methyl-1-(3-chloro-2- |
| | pyridinyl)-3-bromo-1H-pyrazole-5-carboxamide. |
| | white solid, m.p. 233.0-233.7° C. |
| | ¹H NMR (CDCl₃) δ: a first group of peaks: 2.499 (s, 3H, CH₃), 3.090 (s, 3H, CH₃), |
| | 3.247 (s, 3H, CH₃), 5.759 (s, 1H, Pyrazole H), 7.205 (d, J = 1.8 Hz, 1H, Ph H), 7.382 |
| | (dd, J = 8.1 Hz, 4.5 Hz, 1H, Py H), 7.650 (d, J = 1.5 Hz, 1H, Ph H), 7.904 (d, J = 8.1 Hz |
| | 1H, Py H), 8.380 (d, J = 4.5 Hz, 1H, Py H). a second group of peaks: 2.729 (s, 3H, |
| | CH₃), 3.038 (s, 3H, CH₃), 3.503 (s, 3H, CH₃), 6.768 (s, 1H, Pyrazole H), 7.165 (d, |
| | J = 2.1 Hz, 1H, Ph H), 7.323 (dd, J = 8.1 Hz, 4.8 Hz, 1H, Py H), 7.489 (d, J = 2.1 Hz, |
| | 1H, Ph H), 7.853 (d, J = 8.1 Hz 1H, Py H), 8.423 (d, J = 4.8 Hz, 1H, Py H). |
| | ¹H NMR (DMSO) δ: a first group of peaks: 2.506 (s, 3H, CH₃), 3.056 (s, 3H, CH₃), |
| | 3.330 (s, 3H, CH₃), 5.911 (s, 1H, Pyrazole H), 7.697 (s, 1H, Ph H), 7.648 (d, J = 8.1 |
| | Hz, 1H, Py H), 8.003 (s, 1H, Ph H), 8.252 (s, 1H, Py H), 8.509 (s, 1H, Py H). |
| | a second group of peaks: 2.557 (s, 3H, CH₃), 2.978 (s, 3H, CH₃), 3.383 (s, 3H, CH₃), |
| | 7.198 (s, 1H, Pyrazole H), 7.546 (s, 1H, Ph H), 7.606 (d, J = 7.8 Hz, 1H, Py H), 7.846 |
| | (s, 1H, Ph H), 8.225 (s, 1H, Py H), 8.522 (s, 1H, Py H). |
| | ¹H NMR (CD₃OD) δ: a first group of peaks: 2.572 (s, 3H, CH₃), 3.133 (s, 3H, CH₃), |
| | 3.221 (s, 3H, CH₃), 5.908 (s, 1H, Pyrazole H), 7.591 (s, 1H, Ph H), 7.636 (d, J = 7.8 |
| | Hz, 1H, Py H), 7.942 (s, 1H, Ph H), 8.158 (d, J = 7.8 Hz 1H, Py H), 8.501 (d, J = 4.8 |
| | Hz, 1H, Py H). a second group of peaks: 2.736 (s, 3H, CH₃), 3.094 (s, 3H, CH₃), |
| | 3.529 (s, 3H, CH₃), 7.096 (s, 1H, Pyrazole H), 7.478 (s, 1H, Ph H), 7.619 (d, J = 7.8 |
| | Hz, 1H, Py H), 7.750 (s, 1H, Ph H), 8.132 (d, J = 7.8 Hz 1H, Py H), 8.517 (d, J = 4.8 |
| | Hz, 1H, Py H). |
| | LC-MS (Pos M⁺) m/z) calc: 530, found: 530. |
| 12 | N-[2,4-dichloro-6-[(methyl(ethyl)amino)carbonyl]phenyl]-N-methyl-1-(3-chloro- |
| | 2-pyridinyl)-3-bromo-1H-pyrazole-5-carboxamide. |
| | light yellow solid, m.p. 145.9-147.3° C. |
| | ¹H NMR(300 MHz, CDCl₃) δ: a first group of peaks: 0.963 (t, J = 7.2 Hz, 3H, CH₃), |
| | 2.994 (s, 3H, CH₃), 3.430-3.559 (m, 2H, CH₂), 3.504 (s, 3H, CH₃), 6.766 (s, 1H, |
| | Pyrazole H), 7.151 (dd, J = 5.1 Hz, 2.7 Hz, 1H, Ph H), 7.321(dd, J = 8.1 Hz, 4.8 Hz, |
| | 1H, Py H), 7.479 (dd, J = 4.8 Hz, 2.4 Hz, 1H, Ph H), 7.847 (d, J = 7.8 Hz, 1H, Py H), |
| | 8.417 (d, J = 4.5 Hz, 1H, Py H). a second group of peaks: 1.099 (t, J = 6.9 Hz, 3H, |
| | CH₃), 3.036 (s, 3H, CH₃), 3.154-3.202 (m, 2H, CH₂), 3.227 (s, 3H, CH₃), 5.699 (s, |
| | 1H, Pyrazole H), 7.186 (d, J = 7.5 Hz, 1H, Ph H), 7.369 (dd, J = 8.1 Hz, 4.8 Hz, 1H, Py |
| | H), 7.628 (d, J = 6.6 Hz, 1H, Ph H), 7.893 (d, J = 7.8 Hz, 1H, Py H), 8.370 (d, J = 4.5 |
| | Hz, 1H, Py H). |
| | LC-MS (Pos M⁺) m/z) calc: 544, found: 544. |
| 13 | N-[2,4-dichloro-6-[(methyl(isopropyl)amino)carbonyl]phenyl]-N-methyl-1-(3- |
| | chloro-2-pyridinyl)-3-bromo-1H-pyrazole-5-carboxamide. |
| | white solid, m.p. 167.6-168.8° C. |
| | ¹H NMR(300 MHz, CDCl₃) δ: 0.809 (d, J = 6.9 Hz, 3H, CH₃), 1.061 (d, J = 6.9 Hz, 3H, |
| | CH₃), 2.869 (s, 3H, CH₃), 3.503 (s, 3H, CH₃), 3.640-3.704 (m, 1H, CH), 6.763 (s, |
| | 1H, Pyrazole H), 7.139 (dd, J = 5.1 Hz, 2.4 Hz, 1H, Ph H), 7.315 (dd, J = 8.1 Hz, 4.8 |
| | Hz, 1H, Py H), 7.468 (dd, J = 5.1 Hz, 2.4 Hz, 1H, Ph H), 7.837 (d, J = 7.8 Hz, 1H, Py |
| | H), 8.407(d, J = 4.8 Hz, 1H, Py H). |
| | LC-MS (Pos M⁺) m/z) calc: 558, found: 558. |
| 14 | N-[2,4-dichloro-6-[(methyl(cyclopropyl)amino)carbonyl]phenyl]-N-methyl-1-(3- |
| | chloro-2-pyridinyl)-3-bromo-1H-pyrazole-5-carboxamide. |
| | light yellow solid, m.p. 165.5-165.8° C. |
| | ¹H NMR(300 MHz, CDCl₃) δ: 0.407-0.452 (m, 2H, CH₂), 0.813-0.852(m, 2H, CH₂), |
| | 2.412-2.596 (m, 1H, CH), 3.037 (s, 3H, CH₃), 3.515 (s, 3H, CH₃), 6.777 (s, 1H, |
| | Pyrazole H), 7.135 (d, J = 2.4 Hz, 1H, Ph H), 7.329 (dd, J = 8.1 Hz, 4.8 Hz, 1H, Py H), |
| | 7.490 (d, J = 1.8 Hz, 1H, Ph H), 7.839 (d, J = 7.8 Hz, 1H, Py H), 8.415(d, J = 4.8 Hz, |
| | 1H, Py H). |
| | LC-MS (Pos M⁺) m/z) calc: 556, found: 556. |
| 15 | N-[2,4-dichloro-6-[(diethylamino)carbonyl]phenyl]-N-methyl-1-(3-chloro-2- |
| | pyridinyl)-3-bromo-1H-pyrazole-5-carboxamide. |
| | light yellow solid, m.p. 133.2-134.8° C. |
| | ¹H NMR(300 MHz, CDCl₃) δ: a first group of peaks: 0.996 (t, J = 7.2 Hz, 3H, CH₃), |
| | 1.138 (t, J = 7.2 Hz, 3H, CH₃), 3.106-3.357 (m, 4H, 2CH₂), 3.510 (s, 3H, CH₃), 6.765 |
| | (s, 1H, Pyrazole H), 7.160 (d, J = 2.1 Hz, 1H, Ph H), 7.321(dd, J = 8.1 Hz, 4.8 Hz, 1H, |
| | Py H), 7.476 (d, J = 2.1 Hz, 1H, Ph H), 7.845 (d, J = 8.1 Hz, 1H, Py H), 8.413 (d, J = 3.6 |
| | Hz, 1H, Py H). a second group of peaks: 1.210 (t, J = 6.9 Hz, 3H, CH₃), 1.233 (t, |
| | J = 6.9 Hz, 3H, CH₃), 2.851-2.971 (m, 2H, CH₂), 3.288 (s, 3H, CH₃), 3.769-3.838 (m, |
| | 2H, CH₂), 5.750 (s, 1H, Pyrazole H), 7.225 (s, 1H, Ph H), 7.365 (dd, J = 8.1 Hz, 4.8 |
| | Hz, 1H, Py H), 7.632 (s, 1H, Ph H), 7.884 (d, J = 7.8 Hz, 1H, Py H), 8.381 (d, J = 3.6 |
| | Hz, 1H, Py H). |
| | LC-MS (Pos M⁺) m/z) calc: 558, found: 558. |
| 16 | N-[2,4-dichloro-6-[(dipropylamino)carbonyl]phenyl]-N-methyl-1-(3-chloro-2- |
| | pyridinyl)-3-bromo-1H-pyrazole-5-carboxamide. |
| | light yellow solid, m.p. 112.3-114.5° C. |
| | ¹H NMR(300 MHz, CDCl₃) δ: 0.679 (t, J = 7.2 Hz, 6H, 2CH₃), 1.334-1.629 (m, 4H, |
| | 2CH₂), 2.792-3.762 (m, 4H, 2CH₂), 3.505 (s, 3H, CH₃), 6.763 (s, 1H, Pyrazole H), |
| | 7.146 (d, J = 2.1 Hz, 1H, Ph H), 7.321(dd, J = 8.1 Hz, 4.8 Hz, 1H, Py H), 7.471 (d, |
| | J = 2.1 Hz, 1H, Ph H), 7.840(d, J = 8.1 Hz, 1H, Py H), 8.412(d, J = 4.5 Hz, 1H, Py H). |
| | LC-MS (Pos M⁺) m/z) calc: 586, found: 586. |
| 17 | N-[2,4-dichloro-6-[(dimethylamino)carbonyl]phenyl]-N-ethyl-1-(3-chloro-2- |
| | pyridinyl)-3-bromo-1H-pyrazole-5-carboxamide. |
| | light yellow solid, m.p. 150.5-151.7° C. |
| | ¹H NMR(300 MHz, CDCl₃) δ: a first group of peaks: 1.166 (t, J = 7.2 Hz, 3H, CH₃), |
| | 2.514 (s, 3H, CH₃), 2.646 (s, 3H, CH₃), 3.528-3.983 (m, 2H, CH₂), 5.797 (s, 1H, |
| | Pyrazole H), 7.222 (s, 1H, Ph H), 7.368 (dd, J = 8.1 Hz, 4.8 Hz, 1H, Py H), 7.619 (s, |
| | 1H, Ph H), 7.890 (d, J = 8.1 Hz 1H, Py H), 8.382 (d, J = 4.5 Hz, 1H, Py H). |
| | a second group of peaks: 1.208 (t, J = 7.2 Hz, 3H, CH₃), 3.064 (s, 3H, CH₃), 3.080 (s, |
| | 3H, CH₃), 3.791-4.141 (m, 2H, CH₂), 6.726 (s, 1H, Pyrazole H), 7.169 (d, J = 2.1 |
| | Hz, 1H, Ph H), 7.328 (dd, J = 8.1 Hz, 4.5 Hz, 1H, Py H), 7.481 (d, J = 2.1 Hz, 1H, Ph |
| | H), 7.858 (d, J = 8.1 Hz 1H, Py H), 8.414 (d, J = 4.8 Hz, 1H, Py H). |
| | LC-MS (Pos M⁺) m/z) calc: 544, found: 544. |
| 18 | N-[2,4-dichloro-6-[(methyl(ethyl)amino)carbonyl]phenyl]-N-ethyl-1-(3-chloro-2- |
| | pyridinyl)-3-bromo-1H-pyrazole-5-carboxamide. |
| | white solid, m.p. 138.5-141.2° C. |
| | ¹H NMR(300 MHz, CDCl₃) δ: 1.204 (t, J = 7.2 Hz, 3H, CH₃), 1.227 (t, J = 7.2 Hz, 3H, |
| | CH₃), 3.021 (s, 3H, CH₃), 3.468-3.567 (m, 2H, CH₂), 4.059-4.129 (m, 2H, CH₂), |
| | 6.715 (d, J = 2.1 Hz, 1H, Pyrazole H), 7.152 (dd, J = 5.1 Hz, 2.1 Hz, 1H, Ph H), 7.325 |
| | (dd, J = 8.1 Hz, 4.5 Hz, 1H, Py H), 7.467 (dd, J = 5.1 Hz, 2.1 Hz, 1H, Ph H), 7.852 (d, |
| | J = 8.1 Hz 1H, Py H), 8.405 (d, J = 4.8 Hz, 1H, Py H). |
| | LC-MS (Pos M⁺) m/z) calc: 558, found: 558. |
| 19 | N-[2,4-dichloro-6-[(dimethylamino)carbonyl]phenyl]-N-propyl-1-(3-chloro-2- |
| | pyridinyl)-3-bromo-1H-pyrazole-5-carboxamide. |
| | white solid, m.p. 157.3-158.5° C. |
| | ¹H NMR(300 MHz, CDCl₃) δ: 0.837 (t, J = 7.2 Hz, 3H, CH₃), 1.656-1.719 (m, 2H, |
| | CH₂), 3.065 (s, 3H, CH₃), 3.083 (s, 3H, CH₃), 3.699-3.719 (m, 2H, CH₂), 6.705 (s, |
| | 1H, Pyrazole H), 7.162 (d, J = 2.1 Hz, 1H, Ph H), 7.326 (dd, J = 8.1 Hz, 4.5 Hz, 1H, Py |
| | H), 7.471 (d, J = 2.1 Hz, 1H, Ph H), 7.857 (d, J = 8.1 Hz 1H, Py H), 8.408 (d, J = 4.8 Hz, |
| | 1H, Py H). |
| | LC-MS (Pos M⁺) m/z) calc: 558, found: 558. |
| 20 | N-[2,4-dichloro-6-[(methyl(propyl)amino)carbonyl]phenyl]-N-propyl-1-(3- |
| | chloro-2-pyridinyl)-3-bromo-1H-pyrazole-5-carboxamide. |
| | white solid, m.p. 146.1-148.3° C. |
| | ¹H NMR(300 MHz, CDCl₃) δ: 0.781 (t, J = 7.2 Hz, 3H, CH₃), 0.968 (t, J = 7.2 Hz, 3H, |
| | CH₃), 1.596-1.755 (m, 4H, 2 × CH₂), 2.938 (br, 2H, CH₂), 3.031 (s, 3H, CH₃), 3.792- |
| | 3.923 (m, 2H, CH₂), 6.706 (s, 1H, Pyrazole H), 7.133 (d, J = 2.1 Hz, 1H, Ph H), 7.322 |
| | (dd, J = 8.1 Hz, 4.5 Hz, 1H, Py H), 7.465 (d, J = 2.1 Hz, 1H, Ph H), 7.854 (d, J = 8.1 Hz |
| | 1H, Py H), 8.397 (d, J = 4.8 Hz, 1H, Py H). |
| | LC-MS (Pos M⁺) m/z) calc: 586, found: 586. |
| 21 | N-[2,4-dichloro-6-[(dimethylamino)thioxomethyl]phenyl]-N-methyl-1-(3-chloro- |
| | 2-pyridinyl)-3-bromo-1H-pyrazole-5-carboxamide. |
| | light yellow solid, m.p. 222.9-224.9° C. |
| | ¹H NMR(300 MHz, CDCl₃) δ: ¹H NMR (CDCl₃) δ: 3.004 (s, 3H, CH₃), 3.480 (s, 3H, |
| | CH₃), 3.562 (s, 3H, CH₃), 6.763 (s, 1H, Pyrazole H), 7.041 (d, J = 1.8 Hz, 1H, Ph H), |
| | 7.318 (dd, J = 8.1 Hz, 4.5 Hz, 1H, Py H), 7.423 (d, J = 2.1 Hz, 1H, Ph H), 7.853 (d, |
| | J = 8.1 Hz 1H, Py H), 8.416 (d, J = 4.8 Hz, 1H, Py H). |
| | LC-MS (Pos M⁺) m/z) calc: 546, found: 546. |
| 22 | N-[2,4-dichloro-6-[(methyl(isopropyl)amino)thioxomethyl]phenyl]-N-methyl-1- |
| | (3-chloro-2-pyridinyl)-3-bromo-1H-pyrazole-5-carboxamide. |
| | light yellow solid, m.p. 154.5-155.4° C. |
| | ¹H NMR(300 MHz, CDCl₃) δ: 1.117 (d, J = 6.6 Hz, 3H, CH₃), 1.258 (d, J = 6.6 Hz, 3H, |
| | CH₃), 1.961 (s, 3H, CH₃), 3.474 (s, 3H, CH₃), 4.022-4.132 (m, 1H, CH), 6.715 (s, |
| | 1H, Pyrazole H), 7.221 (d, J = 2.1 Hz, 1H, Ph H), 7.319 (dd, J = 7.8 Hz, 4.5 Hz, 1H, Py |
| | H), 7.468 (d, J = 2.1 Hz, 1H, Ph H), 7.852 (d, J = 8.1 Hz, 1H, Py H), 8.428 (d, J = 4.8 |
| | Hz, 1H, Py H); |
| | LC-MS (Pos M⁺) m/z) calc: 574, found: 574. |
| 23 | N-[2,4-dichloro-6-[(diethylamino)thioxomethyl]phenyl]-N-methyl-1-(3-chloro-2- |
| | pyridinyl)-3-bromo-1H-pyrazole-5-carboxamide. |
| | light yellow solid, m.p. 177.0-179.3° C. |
| | ¹H NMR(300 MHz, CDCl₃) δ: 1.041 (t, J = 7.2 Hz, 3H, CH₃), 1.202 (t, J = 7.2 Hz, 3H, |
| | CH₃), 3.037-3.109 (m, 1H, ½CH₂), 3.446-3.579 (m, 2H, CH₂), 3.550 (s, 3H, CH₃), |
| | 4.360-4.448 (m, 1H, ½CH₂), 6.763 (s, 1H, Pyrazole H), 7.056 (d, J = 1.5 Hz, 1H, Ph |
| | H), 7.318 (dd, J = 7.8 Hz, 4.5 Hz, 1H, Py H), 7.404 (d, J = 1.8 Hz, 1H, Ph H), 7.847 (d, |
| | J = 8.1 Hz, 1H, Py H), 8.408 (d, J = 4.8 Hz, 1H, Py H); |
| | LC-MS (Pos M⁺) m/z) calc: 574, found: 574. |
| 24 | N-[2,4-dichloro-6-[(diethylamino)thioxomethyl]phenyl]-N-methyl-1-(3-chloro-2- |
| | pyridinyl)-3-bromo-1H-pyrazole-5-carbothioamide. |
| | light yellow solid, m.p. 198.4-200.4° C. |
| | ¹H NMR(300 MHz, CDCl₃) δ: 1.148 (t, J = 7.2 Hz, 3H, CH₃), 1.271 (t, J = 7.2 Hz, 3H, |
| | CH₃), 3.071-3.190 (m, 1H, ½CH₂), 3.558-3.685 (m, 2H, CH₂), 3.820 (s, 3H, CH₃), |
| | 4.368-4.480 (m, 1H, ½CH₂), 6.562 (s, 1H, Pyrazole H), 7.081 (d, J = 1.8 Hz, 1H, Ph |
| | H), 7.307 (dd, J = 7.8 Hz, 4.5 Hz, 1H, Py H), 7.435 (d, J = 1.8 Hz, 1H, Ph H), 7.848 (d, |
| | J = 8.4 Hz, 1H, Py H), 8.385 (d, J = 4.8 Hz, 1H, Py H); |
| | LC-MS (Pos M⁺) m/z) calc: 590, found: 590. |
| 25 | N-[2-bromo-4-chloro-6-[(methylamino)carbonyl]phenyl]-N-methyl-1-(3-chloro- |
| | 2-pyridinyl)-3-bromo-1H-pyrazole-5-carboxamide. |
| | off-white solid, m.p. >250° C. |
| | ¹H NMR (CDCl₃) δ: δ: 2.870 (d, J = 4.5 Hz, 3H, CH₃), 3.505 (s, 3H, CH₃), 6.103 (br, |
| | 1H, NH), 6.864 (s, 1H, Pyrazole H), 7.321 (dd, J = 8.1 Hz, 4.5 Hz, 1H, Py H), 7.431 |
| | (s, 1H, Ph H), 7.687 (s, 1H, Ph H), 7.879 (d, J = 7.8 Hz 1H, Py H), 8.416 (d, J = 4.5 Hz, |
| | 1H, Py H). |
| | LC-MS (Pos M⁺) m/z) calc: 560, found: 560. |
| 26 | N-[2-bromo-4-chloro-6-[(ethylamino)carbonyl]phenyl]-N-methyl-1-(3-chloro-2- |
| | pyridinyl)-3-bromo-1H-pyrazole-5-carboxamide. |
| | white solid, m.p. 149.8-149.9° C. |
| | ¹H NMR (CDCl₃) δ: 1.089 (t, J = 7.2 Hz, 3H CH₃), 3.317-3.472 (m, 2H, CH₂), 3.512 |
| | (s, 3H, CH₃), 6.015 (br, 1H, NH), 6.862 (s, 1H, Pyrazole H), 7.341 (dd, J = 8.1 Hz, 4.8 |
| | Hz, 1H, Py H), 7.414 (d, J = 1.5 Hz, 1H, Ph H), 7.682 (d, J = 1.5 Hz, 1H, Ph H), 7.838 |
| | (d, J = 8.1 Hz 1H, Py H), 8.408 (d, J = 4.8 Hz, 1H, Py H). |
| | LC-MS (Pos M⁺) m/z) calc: 574, found: 574. |
| 27 | N-[2-bromo-4-chloro-6-[(propylamino)carbonyl]phenyl]-N-methyl-1-(3-chloro-2- |
| | pyridinyl)-3-bromo-1H-pyrazole-5-carboxamide. |
| | white solid, m.p. 230.4-234.7° C. |
| | δ: 0.885 (t, J = 7.2 Hz, 3H CH₃), 1.424-1.567 (m, 2H, CH₂), 3.255-3.461 (m, 2H, |
| | CH₂), 3.512 (s, 3H, CH₃), 6.042 (br, 1H, NH), 6.858 (s, 1H, Pyrazole H), 7.336 (dd, |
| | J = 8.1 Hz, 4.8 Hz, 1H, Py H), 7.403 (d, J = 1.5 Hz, 1H, Ph H), 7.681 (d, J = 1.5 Hz, 1H, |
| | Ph H), 7.864 (d, J = 8.1 Hz 1H, Py H), 8.417 (d, J = 4.5 Hz, 1H, Py H). |
| | LC-MS (Pos M⁺) m/z) calc: 588, found: 588. |
| 28 | N-[2-bromo-4-chloro-6-[(isopropylamino)carbonyl]phenyl]-N-methyl-1-(3- |
| | chloro-2-pyridinyl)-1H-pyrazole-5-carboxamide. |
| | white solid, m.p. >250° C. |
| | ¹H NMR (300 MHz, CDCl₃) δ: 1.250 (d, J = 6.0 Hz, 4H, CH₂), 3.233 (s, H, CH₃), |
| | 4.254 (q, J = 6.6 Hz, 1H, CH), 5.799 (d, J = 6.9 Hz, 1H, NH), 5.967 (s, 1H, Pyrazole |
| | H), 7.107 (d, J = 7.8 Hz, 1H, Ph H), 7.303 (d, J = 8.4 Hz, 1H, Ph H), 7.381-7.445 (m, |
| | 2H, py H), 7.916 (d, J = 7.5 Hz, 1H, ph H), 8.445(d, J = 3.6 Hz, 1H, py H). |
| | LC-MS (Pos M+) m/z) calc: 510, found: 510. |
| 29 | N-[2-bromo-4-chloro-6-[(cyclopropylamino)carbonyl]phenyl]-N-methyl-1-(3- |
| | chloro-2-pyridinyl)-3-bromo-1H-pyrazole-5-carboxamide. |
| | white solid, m.p. 175.1-175.5° C. |
| | δ: 0.424-0.871 (m, 4H, 2 × CH₂), 2.721-2.744 (m, 1H, CH), 3.510 (s, 3H, CH₃), 6.173 |
| | (br, 1H, NH), 6.892 (s, 1H, Pyrazole H), 7.335 (d, J = 2.4 Hz, 1H, Ph H), 7.336 (d, |
| | J = 2.4 Hz, 1H, Ph H), 7.668 (d, J = 2.4 Hz, 1H, Ph H), 7.864 (d, J = 8.1 Hz 1H, Py H), |
| | 8.443 (d, J = 4.5 Hz, 1H, Py H). |
| | LC-MS (Pos M⁺) m/z) calc: 586, found: 586. |
| 30 | N-[2-bromo-4-chloro-6-[(butylamino)carbonyl]phenyl]-N-methyl-1-(3-chloro-2- |
| | pyridinyl)-1H-pyrazole-5-carboxamide. |
| | white solid, m.p. 120.0-125.3° C. |
| | ¹H NMR (300 MHz, CDCl₃) δ: 0.974 (t, J = 6.9 Hz, 3H, CH₃), 1.323-1.419(m, 2H, |
| | CH₂), 1.461-1.572 (m, 2H, CH₂), 3.238 (s, 3H, CH₃), 5.911 (s, 1H, Pyrazole H), |
| | 5.957 (bs, 1H, NH), 6.977(d, J = 7.8 Hz, 1H, Ph H), 7.295 (d, J = 8.7 Hz, 1H, Ph H), |
| | 7.364-7.419 (m, 2H, py H), 7.913 (d, J = 7.5 Hz, 1H, ph H), 8.440 (d, J = 4.2 Hz, 1H, py H). |
| | LC-MS (Pos M+) m/z) calc: 524, found: 524 |
| 31 | N-[2-bromo-4-chloro-6-[(dimethylamino)carbonyl]phenyl]-N-methyl-1-(3- |
| | chloro-2-pyridinyl)-3-bromo-1H-pyrazole-5-carboxamide. |
| | white solid, m.p. 243.5-244.6° C. |
| | ¹H NMR (300 MHz, CDCl₃) δ: 2.712 (s, 3H, CH₃), 3.035 (s, 3H, CH₃), 3.514 (s, |
| | 3H, CH₃), 6.786 (s, 1H, Pyrazole H), 7.202 (d, J = 2.4 Hz, 1H, Ph H), 7.324 (dd, J = 8.1 |
| | Hz, 4.8 Hz, 1H, Py H), 7.656 (d, J = 2.4 Hz, 1H, Ph H), 7.846 (d, J = 8.1 Hz 1H, Py H), |
| | 8.431 (d, J = 4.5 Hz, 1H, Py H). |
| | LC-MS (Pos M⁺) m/z) calc: 574, found: 574. |
| 32 | N-[2-bromo-4-chloro-6-[(methyl(ethyl)amino)carbonyl]phenyl]-N-methyl-1-(3- |
| | chloro-2-pyridinyl)-3-bromo-1H-pyrazole-5-carboxamide. |
| | white solid, m.p. 148.5-150.4° C. |
| | ¹H NMR (300 MHz, CDCl₃) δ: 1.114 (t, J = 7.2 Hz, 3H, CH₃), 2.700 (s, 3H, CH₃), |
| | 3.028 (br, 2H, CH₂), 3.504 (s, 3H, CH₃), 6.798 (s, 1H, Pyrazole H), 7.207 (s, 1H, Ph |
| | H), 7.336 (dd, J = 7.8 Hz, 4.5 Hz, 1H, Py H), 7.661 (s, 1H, Ph H), 7.852 (d, J = 7.8 Hz, |
| | 1H, Py H), 8.435 (d, J = 4.5 Hz, 1H, Py H). |
| | LC-MS (Pos M⁺) m/z) calc: 588, found: 588. |
| 33 | N-[2-bromo-4-chloro-6-[(methyl(propyl)amino)carbonyl]phenyl]-N-methyl-1-(3- |
| | chloro-2-pyridinyl)-3-bromo-1H-pyrazole-5-carboxamide. |
| | light yellow solid, m.p. 159.6-160.1° C. |
| | ¹H NMR (300 MHz, CDCl₃) δ: 0.934 (t, J = 7.2 Hz, 3H, CH₃), 1.576-1.629 (m, 2H, |
| | CH₂), 2.703 (s, 3H, CH₃), 3.230 (br, 2H, CH₂), 3.509 (s, 3H, CH₃), 6.776 (s, 1H, |
| | Pyrazole H), 7.181 (d, J = 2.1 Hz, 1H, Ph H), 7.338 (dd, J = 7.8 Hz, 4.5 Hz, 1H, Py H), |
| | 7.649 (d, J = 2.1 Hz, 1H, Ph H), 7.846 (d, J = 7.8 Hz, 1H, Py H), 8.425 (d, J = 4.5 Hz, |
| | 1H, Py H). |
| | LC-MS (Pos M⁺) m/z) calc: 602, found: 602. |
| 34 | N-[2-bromo-4-chloro-6-[(methyl(isopropyl)amino)carbonyl]phenyl]-N-methyl-1- |
| | (3-chloro-2-pyridinyl)-3-bromo-1H-pyrazole-5-carboxamide. |
| | white solid, m.p. 167.8-170.5° C. |
| | ¹H NMR (CDCl₃) δ: 0.786 (d, J = 6.9 Hz, 3H, CH₃), 1.056 (d, J = 6.9 Hz, 3H, CH₃), |
| | 2.862 (s, 3H, CH₃), 3.527 (s, 3H, CH₃), 4.782-4.869 (m, 1H, CH), 6.785 (s, 1H, |
| | Pyrazole H), 7.169 (dd, J = 4.8 Hz, 2.1 Hz, 1H, Ph H), 7.333 (dd, J = 7.8 Hz, 4.5 Hz, |
| | 1H, Py H), 7.638 (dd, J = 4.8 Hz, 2.1 Hz, 1H, Ph H), 7.834 (d, J = 7.8 Hz, 1H, Py H), |
| | 8.412 (d, J = 4.5 Hz, 1H, Py H). |
| | LC-MS (Pos M⁺) m/z) calc: 602, found: 602. |
| 35 | N-[2-bromo-4-chloro-6-[(methyl(cyclopropyl)amino)carbonyl]phenyl]-N-methyl- |
| | 1-(3-chloro-2-pyridinyl)-3-bromo-1H-pyrazole-5-carboxamide. |
| | yellow solid, m.p. 142.8-143.8° C. |
| | ¹H NMR (CDCl₃) δ: 0.397-0.857 (m, 4H, 2 × CH₂), 2.349-2.372 (m, 1H, CH), 3.033 |
| | (s, 3H, CH₃), 3.527 (s, 3H, CH₃), 6.798 (s, 1H, Pyrazole H), 7.176 (s, 1H, Ph H), |
| | 7.332 (dd, J = 8.1 Hz, 5.1 Hz, 1H, Py H), 7.659 (s, 1H, Ph H), 7.836 (d, J = 8.1 Hz, 1H, |
| | Py H), 8.436 (d, J = 4.5 Hz, 1H, Py H). |
| | LC-MS (Pos M⁺) m/z) calc: 600, found: 600. |
| 36 | N-[2-bromo-4-chloro-6-[(methyl(tert-butyl)amino)carbonyl]phenyl]-N-methyl-1- |
| | (3-chloro-2-pyridinyl)-3-bromo-1H-pyrazole-5-carboxamide. |
| | light yellow solid, m.p. 196.3-197.7° C. |
| | ¹H NMR (CDCl₃) δ: 1.459 (s, 9H, 3 × CH₃), 2.645 (s, 3H, CH₃), 3.515 (s, 3H, CH₃), |
| | 6.776 (s, 1H, Pyrazole H), 7.156 (d, J = 1.8 Hz, 1H, Ph H), 7.320 (dd, J = 8.1 Hz, 5.1 |
| | Hz, 1H, Py H), 7.604 (d, J = 1.8 Hz, 1H, Ph H), 7.846 (d, J = 8.7 Hz, 1H, Py H), 8.418 |
| | (d, J = 4.5 Hz, 1H, Py H). |
| | LC-MS (Pos M⁺) m/z) calc: 616, found: 616. |
| 37 | N-[2-bromo-4-chloro-6-[(diethylamino)carbonyl]phenyl]-N-methyl-1-(3-chloro- |
| | 2-pyridinyl)-3-bromo-1H-pyrazole-5-carboxamide. |
| | yellow solid, m.p. 145.0-146.8° C. |
| | ¹H NMR (CDCl₃) δ: 0.954 (t, J = 7.2 Hz, 3H, CH₃), 1.128 (t, J = 7.2 Hz, 3H, CH₃), |
| | 3.117 (br, 2H, CH₂), 3.483 (br, 2H, CH₂), 3.522 (s, 3H, CH₃), 6.784 (s, 1H, Pyrazole |
| | H), 7.194 (d, J = 2.1 Hz, 1H, Ph H), 7.321 (dd, J = 7.8 Hz, 4.2 Hz, 1H, Py H), 7.641 (d, |
| | J = 2.4 Hz, 1H, Ph H), 7.836 (d, J = 8.7 Hz, 1H, Py H), 8.411 (d, J = 4.5 Hz, 1H, Py H). |
| | LC-MS (Pos M⁺) m/z) calc: 602, found: 602. |
| 38 | N-[2-bromo-4-chloro-6-[(dimethylamino)carbonyl]phenyl]-N-ethyl-1-(3-chloro- |
| | 2-pyridinyl)-3-bromo-1H-pyrazole-5-carboxamide. |
| | light yellow solid, m.p. 136.7-138.2° C. |
| | ¹H NMR (CDCl₃) δ: 0.833 (t, J = 7.2 Hz, 3H, CH₃), 3.058 (s, 3H, CH₃), 3.072 (s, 3H, |
| | CH₃), 4.074 (br, 2H, CH₂), 6.758 (s, 1H, Pyrazole H), 7.206 (d, J = 2.4 Hz, 1H, Ph H), |
| | 7.331 (dd, J = 8.1 Hz, 4.8 Hz, 1H, Py H), 7.658 (d, J = 2.1 Hz, 1H, Ph H), 7.873 (d, |
| | J = 8.7 Hz, 1H, Py H), 8.426 (d, J = 4.5 Hz, 1H, Py H). |
| | LC-MS (Pos M⁺) m/z) calc: 588, found: 588. |
| 39 | N-[2-bromo-4-chloro-6-[(methyl(ethyl)amino)carbonyl]phenyl]-N-ethyl-1-(3- |
| | chloro-2-pyridinyl)-3-bromo-1H-pyrazole-5-carboxamide. |
| | light yellow solid, m.p. 143.6-144.4° C. |
| | ¹H NMR (CDCl₃) δ: 0.903 (t, J = 7.2 Hz, 3H, CH₃), 1.165 (t, J = 7.2 Hz, 3H, CH₃), |
| | 3.098 (s, 3H, CH₃), 3.122 (br, 2H, CH₂), 4.063 (br, 2H, CH₂), 6.749 (s, 1H, Pyrazole |
| | H), 7.188 (dd, J = 2.1 Hz, 5.7 Hz, 1H, Ph H), 7.330 (dd, J = 8.1 Hz, 4.8 Hz, 1H, Py H), |
| | 7.645 (dd, J = 2.1 Hz, 5.7 Hz, 1H, Ph H), 7.851 (d, J = 8.7 Hz, 1H, Py H), 8.418 (d, |
| | J = 4.5 Hz, 1H, Py H). |
| | LC-MS (Pos M⁺) m/z) calc: 602, found: 602. |
| 40 | N-[2-bromo-4-chloro-6-[(diethylamino)carbonyl]phenyl]-N-ethyl-1-(3-chloro-2- |
| | pyridinyl)-3-bromo-1H-pyrazole-5-carboxamide. |
| | off-white solid, m.p. 159.6-160.7° C. |
| | ¹H NMR (CDCl₃) δ: a first group of peaks: 1.185 (t, J = 7.2 Hz, 9H, 3 × CH₃), 2.822 |
| | (br, 2H, CH₂), 3.073 (br, 2H, CH₂), 3.620 (br, 2H, CH₂), 5.631 (s, 1H, Pyrazole H), |
| | 7.370 (dd, J = 8.1 Hz, 4.8 Hz, 1H, Py H), 7.371 (s, 1H, Ph H), 7.846 (s, 1H, Ph H), |
| | 7.849 (d, J = 8.7 Hz 1H, Py H), 8.400 (d, J = 4.2 Hz, 1H, Py H). |
| | a second group of peaks: 1.185 (t, J = 7.2 Hz, 9H, 3 × CH₃), 3.146 (br, 2H, CH₂), 3.706 |
| | (br, 2H, CH₂), 4.063 (br, 2H, CH₂), 6.748 (s, 1H, Pyrazole H), 7.197 (d, J = 2.1 Hz, |
| | 1H, Ph H), 7.332 (dd, J = 8.1 Hz, 4.8 Hz, 1H, Py H), 7.638 (s, 1H, Ph H), 7.903 (d, |
| | J = 8.1 Hz, 1H, Py H), 8.415 (d, J = 4.2 Hz, 1H, Py H). |
| | LC-MS (Pos M⁺) m/z) calc: 616, found: 616. |
| 41 | N-[2-bromo-4-chloro-6-[(dimethylamino)carbonyl]phenyl]-N-propyl-1-(3-chloro- |
| | 2-pyridinyl)-3-bromo-1H-pyrazole-5-carboxamide. |
| | white solid, m.p. 150.0-150.1° C. |
| | ¹H NMR (CDCl₃) δ: 0.859 (t, J = 7.2 Hz, 3H, CH₃), 1.682 (br, 2H, CH₂), 3.025(s, 3H, |
| | CH₃), 3.060 (s, 3H, CH₃), 3.601 (br, 2H, CH₂), 6.737 (s, 1H, Pyrazole H), 7.199 (d, |
| | J = 2.1 Hz, 1H, Ph H), 7.331 (dd, J = 8.1 Hz, 4.8 Hz, 1H, Py H), 7.649 (d, J = 2.1 Hz, |
| | 1H, Ph H), 7.857 (d, J = 8.1 Hz, 1H, Py H), 8.425 (d, J = 4.2 Hz, 1H, Py H). |
| | LC-MS (Pos M⁺) m/z) calc: 602, found: 602. |
| 42 | N-[2-methyl-4-chloro-6-[(methylamino)carbonyl]phenyl]-N-methyl-1-(3-chloro- |
| | 2-pyridinyl)-3-bromo-1H-pyrazole-5-carboxamide. |
| | white solid, m.p. 253.2-255.1° C. |
| | ¹H NMR (CDCl₃) δ: 2.049 (s, 3H, CH₃), 2.900 (d, J = 4.5 Hz, 3H, CH₃), 3.474 (s, 3H, |
| | CH₃), 5.945 (br, 1H, NH), 6.797 (s, 1H, Pyrazole H), 7.305 (s, 1H, Ph H), 7.336 (dd, |
| | J = 8.1 Hz, 4.8 Hz, 1H, Py H), 7.887 (d, J = 7.8 Hz, 4.8 Hz, 1H, Py H), 7.992 (s, 1H, Ph |
| | H), 8.433 (br, 1H, Py H). |
| | LC-MS (Pos M⁺) m/z) calc: 496, found: 496. |
| 43 | N-[2-methyl-4-chloro-6-[(ethylamino)carbonyl]phenyl]-N-methyl-1-(3-chloro-2- |
| | pyridinyl)-3-bromo-1H-pyrazole-5-carboxamide. |
| | white solid, m.p. 237.1-241.9° C. |
| | LC-MS (Pos M⁺) m/z) calc: 510, found: 510. |
| 44 | N-[2-methyl-4-chloro-6-[(dimethylamino)carbonyl]phenyl]-N-methyl-1-(3- |
| | chloro-2-pyridinyl)-3-bromo-1H-pyrazole-5-carboxamide. |
| | light yellow solid, m.p. 191.8-193.0° C. |
| | ¹H NMR (CDCl₃) δ: 2.060 (s, 3H, CH₃), 2.540 (s, 3H, CH₃), 3.060 (s, 3H, CH₃), |
| | 3.484 (s, 3H, CH₃), 6.748 (s, 1H, Pyrazole H), 7.099 (s, 1H, Ph H), 7.332 (dd, J = 8.1 |
| | Hz, 4.8 Hz, 1H, Py H), 7.418 (s, 1H, Ph H), 7.876 (d, J = 8.7 Hz 1H, Py H), 8.394 (d, |
| | J = 4.2 Hz, 1H, Py H). |
| | LC-MS (Pos M⁺) m/z) calc: 510, found: 510. |
| 45 | N-[2-methyl-4-chloro-6-[(methyl(ethyl)amino)carbonyl]phenyl]-N-methyl-1-(3- |
| | chloro-2-pyridinyl)-3-bromo-1H-pyrazole-5-carboxamide. |
| | light yellow solid, m.p. 152.6-153.9° C. |
| | ¹H NMR (CDCl₃) δ: 1.009 (t, J = 7.2 Hz, 3H, CH₃), 2.046 (s, 3H, CH₃), 2.696 (s, 3H, |
| | CH₃), 3.180 (br, 2H, CH₂), 3.479 (s, 3H, CH₃), 6.721 (d, J = 6.9 Hz, 1H, Pyrazole H), |
| | 7.072 (d, J = 2.1 Hz, 1H, Ph H), 7.315 (dd, J = 7.8 Hz, 4.8 Hz, 1H, Py H), 7.341 (d, |
| | J = 2.1 Hz, 1H, Ph H), 7.860 (d, J = 7.8 Hz 1H, Py H), 8.374 (d, J = 4.8 Hz, 1H, Py H). |
| | LC-MS (Pos M⁺) m/z) calc: 524, found: 524. |
| 46 | N-[2-methyl-4-chloro-6-[(methyl(propyl)amino)carbonyl]phenyl]-N-methyl-1-(3- |
| | chloro-2-pyridinyl)-3-bromo-1H-pyrazole-5-carboxamide. |
| | white solid, m.p. 147.9-149.4° C. |
| | ¹H NMR (CDCl₃) δ: 0.953 (t, J = 7.2 Hz, 3H, CH₃), 1.564 (br, 2H, CH₂), 2.285 (s, 3H, |
| | CH₃), 2.676 (br, 2H, CH₂), 3.113 (s, 3H, CH₃), 3.528 (s, 3H, CH₃), 6.742 (s, 1H, |
| | Pyrazole H), 7.065 (d, J = 2.1 Hz, 1H, Ph H), 7.328 (dd, J = 7.8 Hz, 4.8 Hz, 1H, Py H), |
| | 7.404 (d, J = 2.1 Hz, 1H, Ph H), 7.860 (d, J = 7.8 Hz 1H, Py H), 8.388 (d, J = 3.6 Hz, |
| | 1H, Py H). |
| | LC-MS (Pos M⁺) m/z) calc: 538, found: 538. |
| 47 | N-[2-methyl-4-chloro-6-[(dimethylamino)thioxomethyl]phenyl]-N-methyl-1-(3- |
| | chloro-2-pyridinyl)-3-bromo-1H-pyrazole-5-carboxamide. |
| | light yellow solid, m.p. 164.9-166.8° C. |
| | ¹H NMR (CDCl₃) δ: 2.000 (s, 3H, CH₃), 2.206 (s, 3H, CH₃), 3.127 (s, 3H, CH₃), |
| | 3.431 (s, 3H, CH₃), 6.612 (s, 1H, Pyrazole H), 7.010 (s, 1H, Ph H), 7.334 (dd, J = 7.8 |
| | Hz, 4.2 Hz, 1H, Py H), 7.310 (s, 1H, Ph H), 7.870 (d, J = 7.8 Hz 1H, Py H), 8.430 (d, |
| | J = 4.8 Hz, 1H, Py H). |
| | LC-MS (Pos M⁺) m/z) calc: 526, found: 526. |
| 48 | N-[2-methyl-4-bromo-6-[(diethylamino)carbonyl]phenyl]-N-methyl-1-(3-chloro- |
| | 2-pyridinyl)-3-bromo-1H-pyrazole-5-carboxamide. |
| | light yellow solid, m.p. 152.2-153.8° C. |
| | ¹H NMR(300 MHz, CDCl₃) δ: 1.150 (t, J = 7.2 Hz, 3H, CH₃), 1.173 (t, J = 6.9 Hz, 3H, |
| | CH₃), 2.204 (s, 3H, CH₃), 3.089 (br, 2H, CH₂), 3.486 (s, 3H, CH₃), 3.679 (br, 2H, |
| | CH₂), 6.741 (s, 1H, Pyrazole H), 7.240 (s, 1H, Ph H), 7.330 (dd, J = 7.8 Hz, 4.8 Hz, |
| | 1H, Py H), 7.418 (s, 1H, Ph H), 7.855 (d, J = 7.8 Hz, H, Py H), 8.386 (d, J = 4.8 Hz, H, |
| | Py H). |
| | LC-MS (Pos M⁺) m/z) calc: 582, found: 582. |
| 49 | N-[2-methyl-4-bromo-6-[(diethylamino)carbonyl]phenyl]-N-ethyl-1-(3-chloro-2- |
| | pyridinyl)-3-bromo-1H-pyrazole-5-carboxamide. |
| | light yellow solid, m.p. 83.6-85.2° C. |
| | ¹H NMR(300 MHz, CDCl₃) δ: a first group of peaks: 0.911 (t, J = 7.2 Hz, 3H, CH₃), |
| | 1.131 (t, J = 7.2 Hz, 3H, CH₃), 1.261 (t, J = 7.2 Hz, 3H, CH₃), 2.166 (s, 3H, CH₃), |
| | 3.089 (br, 2H, CH₂), 3.113 (br, 2H, CH₂), 3.981 (br, 2H, CH₂), 6.680 (s, 1H, Pyrazole |
| | H), 7.230 (d, J = 1.8 Hz, 1H, Ph H), 7.322 (dd, J = 8.4 Hz, 4.8 Hz, 1H, Py H), 7.364 (d, |
| | J = 1.8 Hz, 1H, Ph H), 7.873(d, J = 8.4 Hz, H, Py H), 8.392(s, 1H, Py H). a |
| | second group of peaks: ¹H NMR(300 MHz, CDCl₃) δ: 0.830 (t, J = 7.2 Hz, 3H, CH₃), |
| | 1.217 (t, J = 6.9 Hz, 3H, CH₃), 1.238 (t, J = 6.9 Hz, 3H, CH₃), 2.307 (s, 3H, CH₃), |
| | 2.849 (br, 2H, CH₂), 3.137 (br, 2H, CH₂), 3.666 (br, 2H, CH₂), 6.198 (s, 1H, Pyrazole |
| | H), 7.338 (s, 1H, Ph H), 7.382 (dd, J = 8.4 Hz, 4.8 Hz, 1H, Py H), 7.464 (s, 1H, Ph H), |
| | 7.873(d, J = 8.4 Hz, H, Py H), 8.372(s, 1H, Py H). |
| | LC-MS (Pos M⁺) m/z) calc: 596, found: 596. |

TABLE 3

| | |
|---|---|
| | (II) |
| 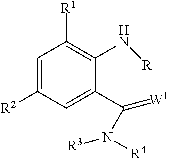 | |

| No | R | R¹ | R² | R³ | R⁴ | W¹ |
|---|---|---|---|---|---|---|
| II-1 | CH₃ | Cl | Cl | H | CH₃ | O |
| II-2 | CH₃ | Cl | Cl | H | CH₂CH₃ | O |
| II-3 | CH₃ | Cl | Cl | H | CH₂CH₂CH₃ | O |
| II-4 | CH₃ | Cl | Cl | H | CH(CH₃)₂ | S |
| II-5 | CH₃ | Cl | Cl | H | CH(CH₃)₂ | O |
| II-6 | CH₃ | Cl | Cl | H | 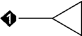 | O |
| II-7 | CH₃ | Cl | Cl | H | CH₂CH₂CH₂CH₃ | O |
| II-8 | CH₃ | Cl | Cl | H | C(CH₃)₃ | O |
| II-9 | CH₂CH₃ | Cl | Cl | H | CH₃ | O |
| II-10 | CH₂CH₃ | Cl | Cl | H | CH₂CH₃ | O |
| II-11 | CH₃ | Cl | Cl | CH₃ | CH₃ | O |
| II-12 | CH₃ | Cl | Cl | CH₃ | CH₂CH₃ | O |
| II-13 | CH₃ | Cl | Cl | CH₃ | CH(CH₃)₂ | O |
| II-14 | CH₃ | Cl | Cl | CH₃ | 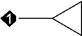 | O |
| II-15 | CH₃ | Cl | Cl | CH₂CH₃ | CH₂CH₃ | O |
| II-16 | CH₃ | Cl | Cl | CH₂CH₂CH₃ | CH₂CH₂CH₃ | O |
| II-17 | CH₂CH₃ | Cl | Cl | CH₃ | CH₃ | O |
| II-18 | CH₂CH₃ | Cl | Cl | CH₃ | CH₂CH₃ | O |
| II-19 | CH₂CH₂CH₃ | Cl | Cl | CH₃ | CH₃ | O |
| II-20 | CH₂CH₂CH₃ | Cl | Cl | CH₃ | CH₂CH₂CH₃ | O |
| II-21 | CH₃ | Cl | Cl | CH₃ | CH₃ | S |
| II-22 | CH₃ | Cl | Cl | CH₃ | CH(CH₃)₂ | S |
| II-23 | CH₃ | Cl | Cl | CH₂CH₃ | CH₂CH₃ | S |
| II-24 | CH₃ | Cl | Cl | CH₂CH₃ | CH₂CH₃ | S |
| II-25 | CH₃ | Br | Cl | H | CH₃ | O |
| II-26 | CH₃ | Br | Cl | H | CH₂CH₃ | O |
| II-27 | CH₃ | Br | Cl | H | CH₂CH₂CH₃ | O |
| II-28 | CH₃ | Br | Cl | H | CH(CH₃)₂ | O |
| II-29 | CH₃ | Br | Cl | H | 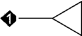 | O |
| II-30 | CH₃ | Br | Cl | H | CH₂CH₂CH₂CH₃ | O |
| II-31 | CH₃ | Br | Cl | CH₃ | CH₃ | O |
| II-32 | CH₃ | Br | Cl | CH₃ | CH₂CH₃ | O |
| II-33 | CH₃ | Br | Cl | CH₃ | CH₂CH₂CH₃ | O |
| II-34 | CH₃ | Br | Cl | CH₃ | CH(CH₃)₂ | O |
| II-35 | CH₃ | Br | Cl | CH₃ | 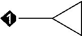 | O |
| II-36 | CH₃ | Br | Cl | CH₃ | C(CH₃)₃ | O |
| II-37 | CH₃ | Br | Cl | CH₂CH₃ | CH₂CH₃ | O |
| II-38 | CH₂CH₃ | Br | Cl | CH₃ | CH₃ | O |
| II-39 | CH₂CH₃ | Br | Cl | CH₃ | CH₂CH₃ | O |
| II-40 | CH₂CH₃ | Br | Cl | CH₂CH₃ | CH₂CH₃ | O |
| II-41 | CH₂CH₂CH₃ | Br | Cl | CH₃ | CH₃ | O |
| II-42 | CH₃ | CH₃ | Cl | H | CH₃ | O |
| II-43 | CH₃ | CH₃ | Cl | H | CH₂CH₃ | O |
| II-44 | CH₃ | CH₃ | Cl | CH₃ | CH₃ | O |
| II-45 | CH₃ | CH₃ | Cl | CH₃ | CH₂CH₃ | O |
| II-46 | CH₃ | CH₃ | Cl | CH₃ | CH₂CH₂CH₃ | O |
| II-47 | CH₃ | CH₃ | Cl | CH₃ | CH₃ | S |
| II-48 | CH₃ | CH₃ | Br | CH₂CH₃ | CH₂CH₃ | O |
| II-49 | CH₂CH₃ | CH₃ | Br | CH₂CH₃ | CH₂CH₃ | O |
| II-50 | CH₃ | CH₃ | Br | CH₃ | CH₃ | O |

TABLE 4

| No. | Physical Properties and ¹H NMR (ppm) |
|---|---|
| II-1 | N-methyl-3,5-dichloro-2-(methylamino)benzamide. light yellow solid, m.p. |
| | 109.7-111.0° C. |
| | ¹H NMR (CDCl₃) δ: 2.794 (s, 3H, CH₃), 2.997 (d, J = 4.5 Hz, 3H, CH₃), 5.898 |
| | (s, 1H, NH), 7.396 (d, J = 2.1 Hz, 1H, Ph H), 7.690 (d, J = 2.1 Hz, 1H, Ph H), |
| | 7.970 (s, 1H, NH). |
| | LC-MS (Pos M⁺) m/z) calc: 233, found: 233. |
| | GC-MS (EI, 70 Ev) (m/z) 232 (50% M⁺), 202 (40% M⁺-30), 173 (100% M⁺-59). |
| II-2 | N-ethyl-3,5-dichloro-2-(methylamino)benzamide. light yellow solid, m.p. |
| | 96.6-98.1° C. |
| | ¹H NMR (CDCl₃) δ: 1.226 (t, J = 7.2 Hz, 3H, CH₃), 2.808 (s, 3H, CH₃), 3.443- |
| | 3.534 (m, 2H, CH₂), 5.353 (bs, 1H, NH), 7.400 (d, J = 2.7 Hz, 1H, Ph H), 7.703 |
| | (d, J = 2.7 Hz, 1H, Ph H), 7.954 (s, 1H, NH). |
| | LC-MS (Pos M⁺) m/z) calc: 247, found: 247. |
| | GC-MS (EI, 70 Ev) (m/z) 246 (40% M⁺), 202 (40% M⁺-44), 173 (100% M⁺-73). |
| II-3 | N-propyl-3,5-dichloro-2-(methylamino)benzamide. |
| | LC-MS (Pos M⁺) m/z) calc: 261, found: 261. |
| II-4 | N-isopropyl-3,5-dichloro-2-(methylamino)benzothioamide. |
| | LC-MS (Pos M⁺) m/z) calc: 277, found: 277. |
| II-5 | N-isopropyl-3,5-dichloro-2-(methylamino)benzamide. |
| | LC-MS (Pos M⁺) m/z) calc: 261, found: 261. |
| II-6 | N-cyclopropyl-3,5-dichloro-2-(methylamino)benzamide. |
| | LC-MS (Pos M⁺) m/z) calc: 259, found: 259. |
| II-7 | N-butyl-3,5-dichloro-2-(methylamino)benzamide. |
| | LC-MS (Pos M⁺) m/z) calc: 275, found: 275. |
| II-8 | N-tert-butyl-3,5-dichloro-2-(methylamino)benzamide. |
| | LC-MS (Pos M⁺) m/z) calc: 275, found: 275. |
| II-9 | N-methyl-3,5-dichloro-2-(ethylamino)benzamide. |
| | LC-MS (Pos M⁺) m/z) calc: 247, found: 247. |
| II-10 | N-ethyl-3,5-dichloro-2-(ethylamino)benzamide. yellow viscous solid. |
| | ¹H NMR (CDCl₃) δ: 0.829 (t, J = 7.2 Hz, 3H, CH₃), 1.116 (t, J = 7.2 Hz, 3H, |
| | CH₃), 3.263-3.307 (m, 2H, CH₂), 3.407-3.497 (m, 2H, CH₂), 6.980 (s, 1H, |
| | NH), 7.225 (d, J = 2.1 Hz, 1H, Ph H), 7.332 (d, J = 2.1 Hz, 1H, Ph H), 7.816 (s, |
| | 1H, NH). |
| | LC-MS (Pos M⁺) m/z) calc: 261, found: 261. |
| II-11 | N,N-dimethyl-3,5-dichloro-2-(methylamino)benzamide. yellow viscous solid. |
| | ¹H NMR (CDCl₃) δ: 2.845 (s, 3H, CH₃), 2.921 (s, 3H, CH₃), 3.111 (s, 3H, |
| | CH₃), 5.306 (s, 1H, NH), 7.033 (d, J = 2.4 Hz, 1H, Ph H), 7.276 (d, J = 2.1 Hz, |
| | 1H, Ph H). |
| | LC-MS (Pos M⁺) m/z) calc: 247, found: 247. |
| | GC-MS (EI, 70 Ev) (m/z) 246 (70% M⁺), 202 (100% M⁺-44), 174 (70% M⁺-72). |
| II-12 | N-methyl-N-ethyl-3,5-dichloro-2-(methylamino)benzamide. |
| | LC-MS (Pos M⁺) m/z) calc: 261, found: 261. |
| II-13 | N-methyl-N-isopropyl-3,5-dichloro-2-(methylamino)benzamide. |
| | LC-MS (Pos M⁺) m/z) calc: 275, found: 275. |
| II-14 | N-methyl-N-cyclopropyl-3,5-dichloro-2-(methylamino)benzamide. |
| | LC-MS (Pos M⁺) m/z) calc: 273, found: 273. |
| II-15 | N,N-diethyl-3,5-dichloro-2-(methylamino)benzamide. yellow viscous solid. |
| | ¹H NMR (CDCl₃) δ: 0.828 (t, J = 7.2 Hz, 3H, CH₃), 1.075 (t, J = 7.2 Hz, 3H, |
| | CH₃), 2.893 (s, 3H, CH₃), 3.221 (q, J = 6.9 Hz, 2H, CH₂), 3.558 (bs, 2H, CH₂), |
| | 5.302 (bs, 1H, NH), 6.982 (d, J = 2.1 Hz, 1H, Ph H), 7.266 (d, J = 2.1 Hz, 1H, Ph |
| | H). |
| | LC-MS (Pos M⁺) m/z) calc: 275, found: 275. |
| | GC-MS (EI, 70 Ev) (m/z) 274 (60% M⁺), 202 (100% M⁺-72), 174 (50% M⁺-100). |
| II-16 | N,N-dipropyl-3,5-dichloro-2-(methylamino)benzamide. |
| | LC-MS (Pos M⁺) m/z) calc: 303, found: 303. |
| II-17 | N,N-dimethyl-3,5-dichloro-2-(ethylamino)benzamide. |
| | LC-MS (Pos M⁺) m/z) calc: 261, found: 261. |
| II-18 | N-methyl-N-ethyl-3,5-dichloro-2-(ethylamino)benzamide. |
| | LC-MS (Pos M⁺) m/z) calc: 275, found: 275. |
| II-19 | N,N-dimethyl-3,5-dichloro-2-(propylamino)benzamide. |
| | LC-MS (Pos M⁺) m/z) calc: 275, found: 275. |
| II-20 | N-methyl-N-propyl-3,5-dichloro-2-(propylamino)benzamide. |
| | LC-MS (Pos M⁺) m/z) calc: 303, found: 303. |
| II-21 | N,N-dimethyl-3,5-dichloro-2-(methylamino)benzothioamide. |
| | LC-MS (Pos M⁺) m/z) calc: 263, found: 263. |
| II-22 | N-methyl-N-isopropyl-3,5-dichloro-2-(methylamino)benzothioamide. |
| | LC-MS (Pos M⁺) m/z) calc: 291, found: 291. |
| II-23 | N,N-diethyl-3,5-dichloro-2-(methylamino)benzothioamide. |
| | LC-MS (Pos M⁺) m/z) calc: 291, found: 291. |
| II-24 | N,N-diethyl-3,5-dichloro-2-(ethylamino)benzothioamide. |
| | LC-MS (Pos M⁺) m/z) calc: 305, found: 305. |
| II-25 | N-methyl-3-bromo-5-chloro-2-(methylamino)benzamide. |
| | LC-MS (Pos M⁺) m/z) calc: 277, found: 277. |
| II-26 | N-ethyl-3-bromo-5-chloro-2-(methylamino)benzamide. |
| | LC-MS (Pos M⁺) m/z) calc: 291, found: 291. |
| II-27 | N-propyl-3-bromo-5-chloro-2-(methylamino)benzamide. |
| | LC-MS (Pos M⁺) m/z) calc: 305, found: 305. |
| II-28 | N-isopropyl-3-bromo-5-chloro-2-(methylamino)benzamide. |
| | LC-MS (Pos M⁺) m/z) calc: 305, found: 305. |
| II-29 | N-cyclopropyl-3-bromo-5-chloro-2-(methylamino)benzamide. |
| | LC-MS (Pos M⁺) m/z) calc: 303, found: 303. |
| II-30 | N-butyl-3-bromo-5-chloro-2-(methylamino)benzamide. |
| | LC-MS (Pos M⁺) m/z) calc: 319, found: 319. |
| II-31 | N,N-dimethyl-3-bromo-5-chloro-2-(methylamino)benzamide. |
| | LC-MS (Pos M⁺) m/z) calc: 291, found: 291. |
| II-32 | N-methyl-N-ethyl-3-bromo-5-chloro-2-(methylamino)benzamide. |
| | LC-MS (Pos M⁺) m/z) calc: 305, found: 305. |
| II-33 | N-methyl-N-propyl-3-bromo-5-chloro-2-(methylamino)benzamide. |
| | LC-MS (Pos M⁺) m/z) calc: 319, found: 319. |
| II-34 | N-methyl-N-isopropyl-3-bromo-5-chloro-2-(methylamino)benzamide. |
| | LC-MS (Pos M⁺) m/z) calc: 319, found: 319. |
| II-35 | N-methyl-N-cyclopropyl-3-bromo-5-chloro-2-(methylamino)benzamide. |
| | LC-MS (Pos M⁺) m/z) calc: 317, found: 317. |
| II-36 | N-methyl-N-tert-butyl-3-bromo-5-chloro-2-(methylamino)benzamide. |
| | LC-MS (Pos M⁺) m/z) calc: 333, found: 333. |
| II-37 | N,N-diethyl-3-bromo-5-chloro-2-(methylamino)benzamide. |
| | LC-MS (Pos M⁺) m/z) calc: 319, found: 319. |
| II-38 | N,N-dimethyl-3-bromo-5-chloro-2-(ethylamino)benzamide. |
| | LC-MS (Pos M⁺) m/z) calc: 305, found: 305. |
| II-39 | N-methyl-N-ethyl-3-bromo-5-chloro-2-(ethylamino)benzamide. |
| | LC-MS (Pos M⁺) m/z) calc: 319, found: 319. |
| II-40 | N,N-diethyl-3-bromo-5-chloro-2-(ethylamino)benzamide. |
| | LC-MS (Pos M⁺) m/z) calc: 333, found: 333. |
| II-41 | N,N-dimethyl-3-bromo-5-chloro-2-(propylamino)benzamide. |
| | LC-MS (Pos M⁺) m/z) calc: 319, found: 319. |
| II-42 | N-methyl-3-methyl-5-chloro-2-(methylamino)benzamide. |
| | LC-MS (Pos M⁺) m/z) calc: 213, found: 213. |
| II-43 | N-ethyl-3-methyl-5-chloro-2-(methylamino)benzamide. |
| | LC-MS (Pos M⁺) m/z) calc: 227, found: 227. |
| II-44 | N,N-dimethyl-3-methyl-5-chloro-2-(methylamino)benzamide. |
| | LC-MS (Pos M⁺) m/z) calc: 227, found: 227. |
| II-45 | N-methyl-N-ethyl-3-methyl-5-chloro-2-(methylamino)benzamide. |
| | LC-MS (Pos M⁺) m/z) calc: 241, found: 241. |
| II-46 | N-methyl-N-propyl-3-methyl-5-chloro-2-(methylamino)benzamide. |
| | LC-MS (Pos M⁺) m/z) calc: 255, found: 255. |
| II-47 | N,N-dimethyl-3-methyl-5-chloro-2-(methylamino)benzothioamide. |
| | LC-MS (Pos M⁺) m/z) calc: 243, found: 243. |
| II-48 | N,N-diethyl-3-methyl-5-bromo-2-(methylamino)benzamide |
| | LC-MS (Pos M⁺) m/z) calc: 299, found: 299. |
| II-49 | N,N-diethyl-3-methyl-5-bromo-2-(ethylamino)benzamide. |
| | LC-MS (Pos M⁺) m/z) calc: 313, found: 313. |
| II-50 | N,N-dimethyl-3-methyl-5-bromo-2-(methylamino)benzamide. |
| | LC-MS (Pos M⁺) m/z) calc: 271, found: 271. |

Compounds of Formula (I) of this invention can be prepared as outlined in Scheme 1; (II) in Scheme 1-1 can be prepared as outlined in Scheme 2; (III) in Scheme 1-1 can be prepared as outlined in Scheme 3; (IV) in Scheme 1-2 can be prepared as outlined in Scheme 4; (V) and (VII) in Scheme 1-2 can be commercially available or prepared with references; (VIII) in Scheme 2 can be prepared as outlined in Scheme 5; (X) in Scheme 3, (XII) in Scheme 4 and Scheme 5 can be commercially available or prepared with references; Unless specially indicated, substituents in Scheme 1 to Scheme 5 are the same as above definitions, wherein L is a leaving group, which is chlorine or bromine.

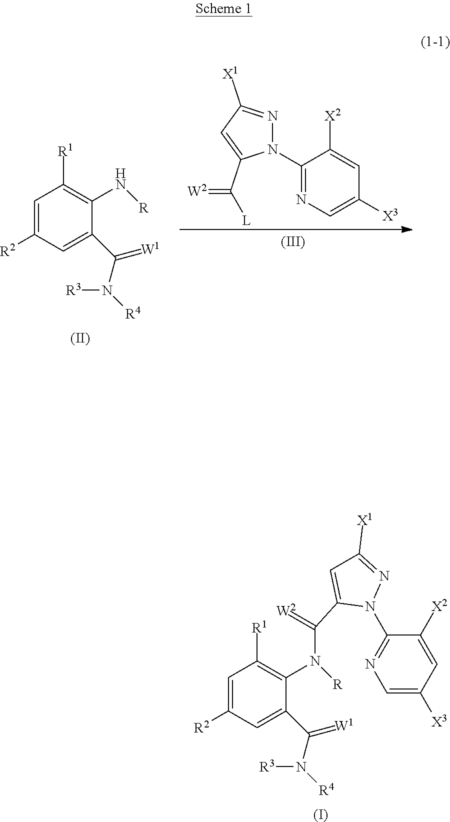

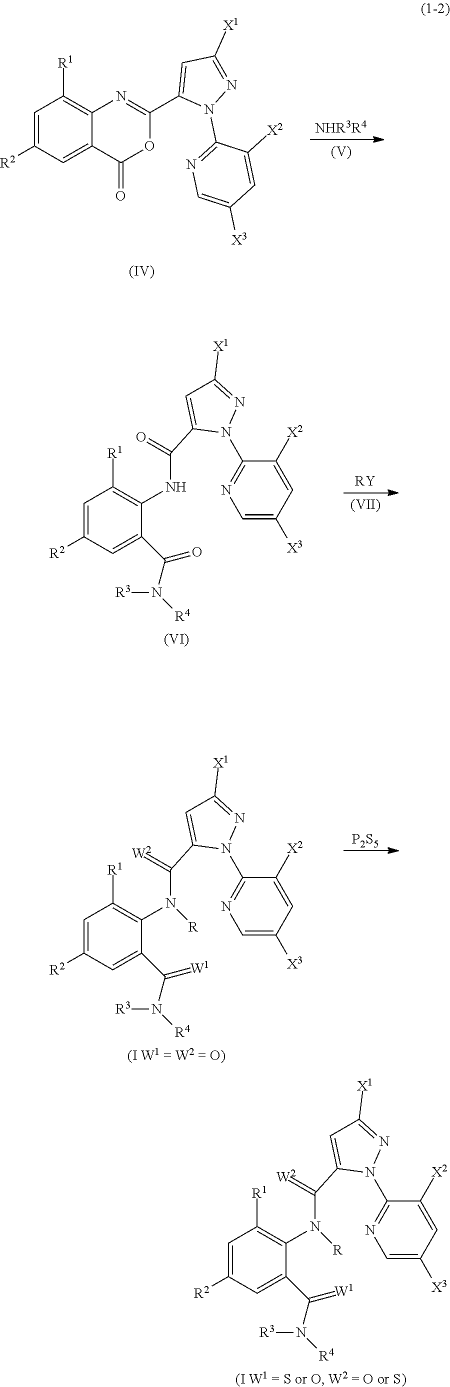

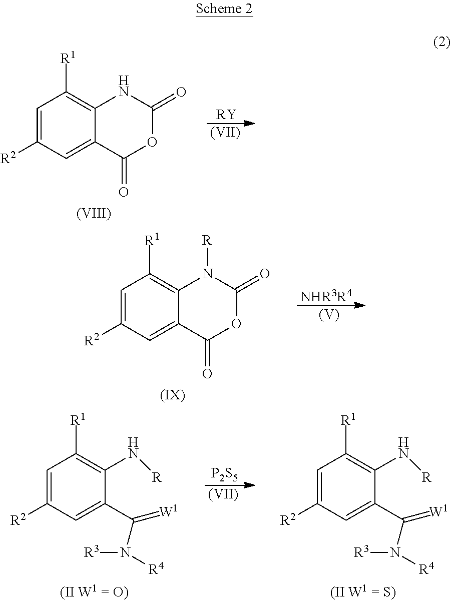

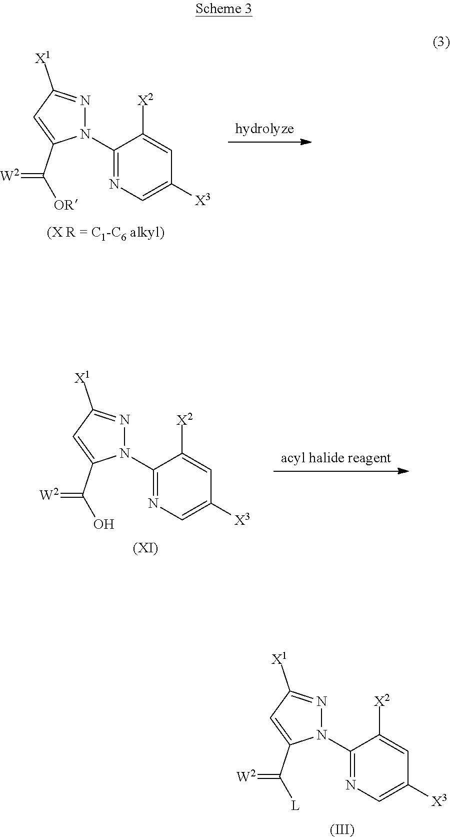

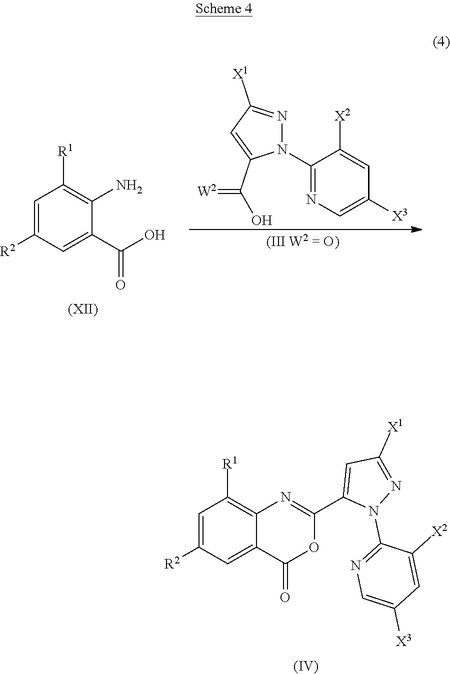

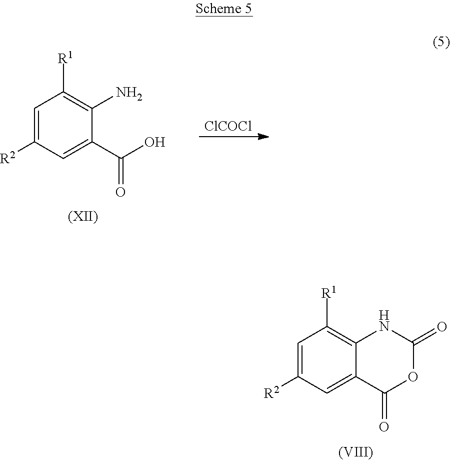

The compounds of Formula (I) can be prepared as outlined in Scheme 1-1: In an appropriate solvent or a mixture of any two appropriate solvents, at certain temperatures from −10° C. to the reflux temperature of the solvent used, reacting a compound of Formula (II) with a compound of Formula (III) to afford a compound of Formula (I), the addition of an appropriate base can accelerate or facilitate the reaction, the appropriate solvent is selected from tetrahydrofuran, 1,4-dioxane, acetone, benzene, toluene, xylene, dichloromethane, dichloroethane, chloroform, tetrachloromethane, hexane, ethyl acetate, acetonitrile, dimethyl sulfoxide or N,N-dimethylformamide, the appropriate base is selected from triethylamine, N,N-dimethylaniline, pyridine, sodium hydroxide, potassium hydroxide, sodium carbonate, potassium carbonate, sodium hydride, sodium methoxide, sodium tert-butoxide, potassium tert-butoxide or sodium bicarbonate.

The compounds of Formula (I) can also be prepared as outlined in Scheme 1-2: In an appropriate solvent or a mixture of any two appropriate solvents, at certain temperatures from −15° C. to the reflux temperature of the solvent used, reacting a compound of Formula (IV) with a compound of Formula (V) to afford a compound of Formula (VI), the appropriate solvent is selected from water, tetrahydrofuran, 1,4-dioxane, acetone, benzene, toluene, xylene, dichloromethane, dichloroethane, chloroform, tetrachloromethane, hexane, ethyl acetate, acetonitrile, dimethyl sulfoxide or N,N-dimethylformamide; In an appropriate solvent or a mixture of any two appropriate solvents, at certain temperatures from −15° C. to the reflux temperature of the solvent used, in the presence of an appropriate base, reacting a compound of Formula (VI) with a compound of Formula (VII) to afford a compound of Formula (I W¹=W²=O), the appropriate solvent is selected from tetrahydrofuran, 1,4-dioxane, acetone, benzene, toluene, xylene, dichloromethane, dichloroethane, chloroform, tetrachloromethane, hexane, ethyl acetate, acetonitrile, dimethyl sulfoxide or N,N-dimethylformamide, the appropriate base is selected from triethylamine, N,N-dimethylaniline, pyridine, methylpyridine, sodium hydroxide, potassium hydroxide, sodium carbonate, potassium carbonate, sodium hydride, sodium methoxide, sodium tert-butoxide, potassium tert-butoxide or sodium bicarbonate; In an appropriate solvent or a mixture of any two appropriate solvents, at certain temperatures from −15° C. to the reflux temperature of the solvent used, reacting a compound of Formula (I W¹=W²=O) with P₂S₅ to afford a compound of Formula (I W¹=S or O, W²=O or S), the appropriate solvent is selected from tetrahydrofuran, 1,4-dioxane, acetone, benzene, toluene, xylene, dichloromethane, dichloroethane, chloroform, tetrachloromethane, hexane, ethyl acetate, acetonitrile, dimethyl sulfoxide or N,N-dimethylformamide.

The compounds of Formula (II) can be prepared as outlined in Scheme 2: In an appropriate solvent or a mixture of any two appropriate solvents, at certain temperatures from −15° C. to the reflux temperature of the solvent used, in the presence of an appropriate base, reacting a compound of Formula (VIII) with a compound of Formula (VII) to afford a compound of Formula (IX), the appropriate solvent is selected from tetrahydrofuran, 1,4-dioxane, acetone, benzene, toluene, xylene, dichloromethane, dichloroethane, chloroform, tetrachloromethane, hexane, ethyl acetate, acetonitrile, dimethyl sulfoxide or N,N-dimethylformamide, the appropriate base is selected from triethylamine, N,N-dimethylaniline, pyridine, methylpyridine, sodium hydroxide, potassium hydroxide, sodium carbonate, potassium carbonate, sodium hydride, sodium methoxide, sodium tert-butoxide, potassium tert-butoxide or sodium bicarbonate; In an appropriate solvent or a mixture of any two appropriate solvents, at certain temperatures from −15° C. to the reflux temperature of the solvent used, in the presence of an appropriate base, reacting a compound of Formula (IX) with a compound of Formula (V) to afford a compound of Formula (II W¹=O), the appropriate solvent is selected from water, tetrahydrofuran, 1,4-dioxane, benzene, toluene, xylene, dichloromethane, dichloroethane, chloroform, tetrachloromethane, hexane, ethyl acetate, acetonitrile, dimethyl sulfoxide or N,N-dimethylformamide; In an appropriate solvent or a mixture of any two appropriate solvents, at certain temperatures from −15° C. to the reflux temperature of the solvent used, reacting a compound of Formula (II W¹=O) with P₂S₅ to afford a compound of Formula (II W¹=S), the appropriate solvent is selected from tetrahydrofuran, 1,4-dioxane, benzene, toluene, xylene, dichloromethane, dichloroethane, chloroform, tetrachloromethane, hexane, ethyl acetate, acetonitrile, dimethyl sulfoxide or N,N-dimethylformamide.

The compounds of Formula (III) can be prepared as outlined in Scheme 3: In an appropriate solvent or a mixture of any two appropriate solvents, at certain temperatures from 25° C. to the reflux temperature of the solvent used, in the presence of an appropriate base, routine hydrolysis of a compound of Formula (X) and then treating the reaction mixture with dilute acid solution such as dilute hydrochloric acid solution to afford a compound of Formula (XI), the appropriate solvent is selected from water, ethanol, methanol, tetrahydrofuran, 1,4-dioxane, benzene, toluene, xylene, dichloromethane, dichloroethane, chloroform, tetrachloromethane, hexane, ethyl acetate, acetonitrile, dimethyl sulfoxide or N,N-dimethylformamide, the appropriate base is selected from triethylamine, N,N-dimethylaniline, pyridine, methylpyridine, sodium hydroxide, potassium hydroxide, sodium carbonate, potassium carbonate, sodium hydride, sodium methoxide, sodium tert-butoxide, potassium tert-butoxide or sodium bicarbonate; In solvent-free or an appropriate solvent, at certain temperatures from 15° C. to the reflux temperature of the solvent used, reacting a compound of Formula (XI) with an appropriate acyl halide reagent to afford a compound of Formula (III), the appropriate solvent is selected from tetrahydrofuran, 1,4-dioxane, benzene, toluene, xylene, dichloromethane, dichloroethane, chloroform, tetrachloromethane, hexane, ethyl acetate or acetonitrile, the appropriate acyl halide reagent is selected from sulfoxide chloride, phosgene, phosphorus trichloride, phosphorus tribromide, phosphorus pentachloride, phosphorus pentabromide or oxalyl chloride etc.

The compounds of Formula (IV) can be prepared as outlined in Scheme 4: In an appropriate solvent or a mixture of any two appropriate solvents, at certain temperatures from −15° C. to the reflux temperature of the solvent used, in the presence of an appropriate base, treating a mixture of a compound of Formula (XII) and a compound of Formula (III W²=O) with methylsulfonyl chloride to afford a compound of Formula (IV), the appropriate solvent is selected from tetrahydrofuran, 1,4-dioxane, acetone, toluene, xylene, acetonitrile, dichloromethane, dichloroethane or N,N-dimethylformamide, the appropriate base is pyridine or methylpyridine.

The compounds of Formula (VIII) can be prepared as outlined in Scheme 5: In an appropriate solvent or a mixture of any two appropriate solvents, at −15° C. to 25° C., reacting a compound of Formula (XII) with phosgene, diphosgene or triphosgene for 2 to 10 hours, and then at certain temperatures from 15° C. to the reflux temperature for 2 to 10 hours, to afford a compound of Formula (VIII), the appropriate solvent is selected from tetrahydrofuran, acetone, 1,4-dioxane, benzene, toluene, xylene, dichloromethane, dichloroethane, chloroform, tetrachloromethane, hexane, ethyl acetate or acetonitrile.

The synthesis methods are described in more detail in the following examples.

Compounds of Formula (I) provided by this invention have broad-spectrum biological activity at a dosage of 7.5 to 2250 g of active ingredient per hectare, and can be used for controlling pests, some compounds have excellent pest control effect and can have excellent control effect at very low dosages.

Compounds of Formula (I) provided by this invention have biological activity, and some compounds have excellent biological activity, especially in the control of pests in agriculture, horticulture, flowers and health. The insect pests described herein include, but are not limited to, and in no way limit this invention.

Insect Pests and Mites:

Homoptera pests such as leafhopper (Cicadellidae), planthoppers (Delphacidae), aphids (Aphididae) and mealybugs (Hemiptera: Coccomorpha: Pseudococcidae), etc.; Lepidoptera pests such as armyworm (Mythimna separata (Walker)), stem borers, common cutworm (Spodoptera litura (Fabricius)), diamondback moth (Plutella xylostella (Linnaeus)), beet armyworm (Spodoptera exigua (Hiibner)), cabbage looper (Trichoplusia ni), cabbageworm caterpillar (Pieris rapae), etc.; Hymenoptera pests such as larva of Sawfly (Tenthredinidae), etc.; Diptera pests such as aedine mosquitoes (Aedes), Culex (Culicidae), flies (Cyclorrhapha), etc.; Hemiptera pests such as whitefly and other Aleyrodidae, etc., Thysanoptera pests such as trips (Thripidae); Acari pests such as cotton spider mites (Tetranychus cinnabarinus), Citrus Red Mite (Panonychus citri (McGregor)), Kanzawa spider mite (Tetranychus kanzawai Kishida), apple leaf mites (Tetranychus viennensis, Panonychus ulmi, Bryobia rubrioculus, etc.), citrus rust mites (Phyllocoptruta oleivora), bulb mites (Rhizoglyphus hyacinthi, etc), two-point spider mites (Tetranychus urticae Koch.), etc.

Particularly, Compounds of Formula (I) still have excellent activity at low concentrations against Homoptera pests such as aphids and mealybugs, etc., Lepidoptera pests such as armyworm, diamondback moth, stem borers, common cutworm, etc., Hemiptera pests such as whitefly, etc. and Thysanoptera pests such as trips.

Due to the positive properties, the above compounds can be advantageously used to protect important crops in agriculture and horticulture, livestock and breeding animals, as well as the environment often visited by humans from pests.

To achieve the desired effect, the dosage of the compound will vary due to such factors as the compound used, the pre-protector, the species of pest to be controlled, the degree of infection, climatic conditions, the application method, and the formulation used, etc.

This invention also relates to an insecticidal composition comprising between 0.5-99% a compound of general Formula (I) as the active ingredient. The insecticidal composition also includes acceptable carriers in agriculture, forestry and health.

When the compound of Formula (I) of this invention is used alone, it is effective for pest control, and they can also be used together with other biochemical substances, including other insecticides, fungicides, herbicides, plant growth regulators, acaricides or fertilizers, which can produce additional advantages and effects.

Formulations using the compounds of Formula (I) provided by this invention as active ingredients can be made into any kind of desired formulations, such as such as dry compressed granules, easy flowing mixtures, granules, wettable powders, water dispersible granules, emulsifiable concentrates, powders, powdery concentrates, microemulsions, suspensions, emulsions, water emulsions, soluble liquid agents, water agents, dispersible liquid agents. Suitable additives include carriers (diluents) and other auxiliary agents such as spreading agents, emulsifiers, wetting agents, dispersants, adhesive and decomposing agent. These formulations contain compounds of this invention mixed with inert, pharmacologically acceptable solid or liquid diluents.

The composition example of this invention can also be made into any desired formulations, such as dry compressed granules, easy flowing mixtures, granules, wettable powders, water dispersible granules, emulsifiable concentrates, powders, powdery concentrates, microemulsions, suspensions, emulsions, water emulsions, soluble liquid agents, water agents, dispersible liquid agents. Suitable additives include carriers (diluents) and other auxiliary agents such as spreading agents, emulsifiers, wetting agents, dispersants, adhesive and decomposing agent. These formulations contain compounds of this invention mixed with inert, pharmacologically acceptable solid or liquid diluents.

It shall be noted that variations and changes are permitted within the claimed scopes of this invention.

This invention is further explained with following examples, and the yields in the examples are not optimized.

### DESCRIPTION OF EMBODIMENTS

### Example 1 Preparation of Compound 1 in Table 1 and Compound II-1 in Table 3

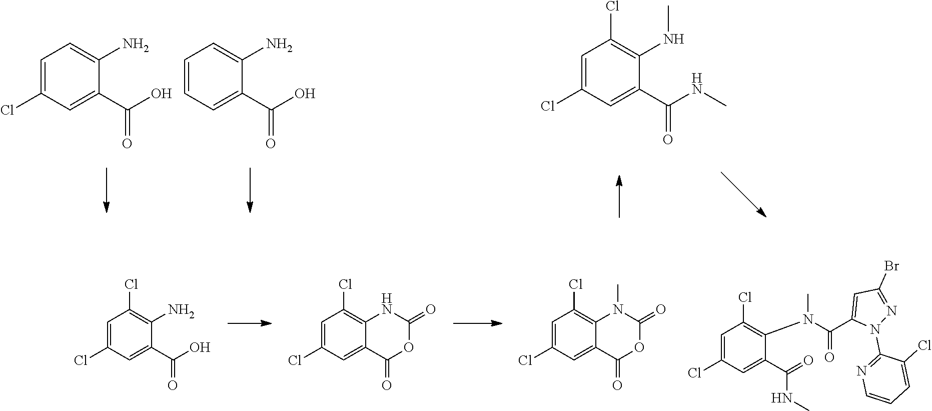

### 2-amino-3,5-dichlorobenzoic Acid (Method 1)

A mixture of 2-amino-5-chlorobenzoic acid (0.10 mol) and N-chlorosuccinimide (0.13 mol) or 2-aminobenzoic acid (0.10 mol) and N-chlorosuccinimide (0.25 mol) in glacial acetic acid (150 mL) was heated to reflux for 4-8 hours until the reaction was complete. The reaction mixture was cooled to room temperature, the title compound 2-amino-3,5-dichlorobenzoic acid in greater than 85% yield was afforded by routine post-treatments such as filtration, water washing and drying.

### 2-amino-3,5-dichlorobenzoic Acid (Method 2)

Excess chlorine was introduced into a solution of 2-amino-5-chlorobenzoic acid (0.10 mol) or 2-aminobenzoic acid (0.10 mol) in glacial acetic acid (150 mL) under room temperature and stirring conditions, the reaction mixture was then stirred at temperatures from 25° C. to the reflux temperature until the reaction was complete. the title compound 2-amino-3,5-dichlorobenzoic acid in greater than 80% yield was afforded by routine post-treatments.

### 6,8-dichloro-2H-3,1-benzooxazine-2,4(1H)-dione (Method 1)

To a solution of 2-amino-3,5-dichlorobenzoic acid (0.05 mol) in tetrahydrofuran (50 mL) was slowly added dropwise a solution of phosgene (0.07 mol) in tetrahydrofuran under −10 to −5° C. and stirring conditions. The reaction mixture was stirred at room temperature for 2-5 hours, then heated to reflux and continued stirring 2-5 hours until the reaction was complete. The reaction mixture was cooled to room temperature, filtered to afford the title compound; the filtrate was concentrated to a ⅓ of the volume, cooled to afford a second crop of the title compound, which was also collected by filtration. The combined total yield was greater than 85%.

### 6,8-dichloro-2H-3,1-benzooxazine-2,4(1H)-dione (Method 2)

To a solution of 2-amino-3,5-dichlorobenzoic acid (0.05 mol) in tetrahydrofuran (80 mL) was slowly introduced phosgene (0.07 mol) under −10 to −5° C. and stirring conditions. The reaction mixture was stirred at room temperature for 2-4 hours, then heated to reflux and continued stirring 3-5 hours. The reaction mixture was cooled to room temperature, filtered to afford the title compound; the filtrate was concentrated to a ⅓ of the volume, cooled to afford a second crop of the title compound, which was also collected by filtration. The combined total yield was greater than 85%.

### 6,8-dichloro-2H-3,1-benzooxazine-2,4(1H)-dione (Method 3)

To a solution of 2-amino-3,5-dichlorobenzoic acid (0.05 mol) in tetrahydrofuran (80 mL) was added a catalytic amount of pyridine, and then was slowly added in batches triphosgene (0.03 mol) under −10 to −5° C. and stirring conditions. The reaction mixture was stirred at room temperature for 2-5 hours, then heated to reflux and continued stirring 2-5 hours. The reaction mixture was cooled to room temperature, filtered to afford the title compound; the filtrate was concentrated to a ⅓ of the volume, cooled to afford a second crop of the title compound, which was also collected by filtration. The combined total yield was greater than 65%.

### 6,8-dichloro-1-methyl-2H-3,1-benzooxazine-2,4(1H)-dione

To a mixture of 6,8-dichloro-2H-3,1-benzooxazine-2,4(1H)-dione (0.02 mol) and sodium hydride (0.03 mol) in N,N-dimethylformamide (25 mL) was slowly added dropwise iodomethane (0.03 mol) under room temperature and stirring conditions. the reaction mixture was continued stirring 6-10 hours until the reaction was complete. the title compound in greater than 70% yield was afforded by routine post-treatments such as the reaction mixture extracted with ethyl acetate, washed with ice water, dried, concentrated and purified, etc.

### N-methyl-3,5-dichloro-2-(methylamino)benzamide

A mixture of 6,8-dichloro-1-methyl-2H-3,1-benzooxazine-2,4(1H)-dione (0.01 mol) and methylamine aqueous solution (33%, 10 mL) was stirred at room temperature for 2-6 hours until the reaction was complete. the title compound in about 70% yield was afford by routine post-treatments such as the reaction mixture extracted with ethyl acetate, washed with ice water, dried, concentrated and purified, etc.

### N-[2,4-dichloro-6-[(methylamino)carbonyl]phenyl]-N-methyl-1-(3-chloro-2-pyridinyl)-3-bromo-1H-pyrazole-5-carboxamide

A mixture of N-methyl-3,5-dichloro-2-(methylamino)benzamide (5.0 mmol) and 3-bromo-1-(3-chloro-2-pyridinyl)-1H-pyrazole-5-carbonyl chloride (5.5 mmol) in toluene (20 mL) or a mixture of toluene (5 mL) and xylene (15 mL) were refluxed for 2-6 hours with stirring until the reaction was complete. The reaction mixture was cooled to room temperature, the resulting crude products were collected by filtration and recrystallized from petroleum ether and ethyl acetate to afford the title compound in greater than 80% yield.

### Example 2 Preparation of Compound 2 in Table 1 and Compound II-2 in Table 3

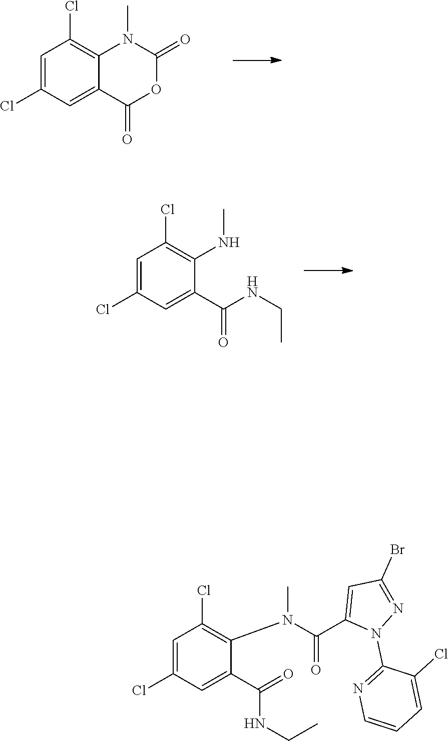 [SMILES: Cn1c(=O)oc(=O)c2cc(Cl)cc(Cl)c21 ; CCNC(=O)c1cc(Cl)cc(Cl)c1NC ; CCNC(=O)c1cc(Cl)cc(Cl)c1N(C)C(=O)c1cc(Br)nn1-c1ncccc1Cl]

### N-ethyl-3,5-dichloro-2-(methylamino)benzamide

A mixture of 6,8-dichloro-1-methyl-2H-3,1-benzooxazine-2,4(1H)-dione (0.01 mol) and ethylamine aqueous solution (65%, 10 mL) was stirred at room temperature for 2-6 hours until the reaction was complete. the title compound in about 70% yield was afford by routine post-treatments such as the reaction mixture extracted with ethyl acetate, washed with ice water, dried, concentrated and purified, etc.

### N-[2,4-dichloro-6-[(ethylamino)carbonyl]phenyl]-N-methyl-1-(3-chloro-2-pyridinyl)-3-bromo-1H-pyrazole-5-carboxamide

A mixture of N-ethyl-3,5-dichloro-2-(methylamino)benzamide (5.0 mmol) and 3-bromo-1-(3-chloro-2-pyridinyl)-1H-pyrazole-5-carbonyl chloride (5.5 mmol) in toluene (20 mL) or a mixture of toluene (5 mL) and xylene (15 mL) were refluxed for 2-6 hours with stirring until the reaction was complete. The reaction mixture was cooled to room temperature, the resulting crude products were collected by filtration and recrystallized from petroleum ether and ethyl acetate to afford the title compound in greater than 80% yield.

### Example 3 Preparation of Compound 9 in Table 1 and Compound II-9 in Table 3

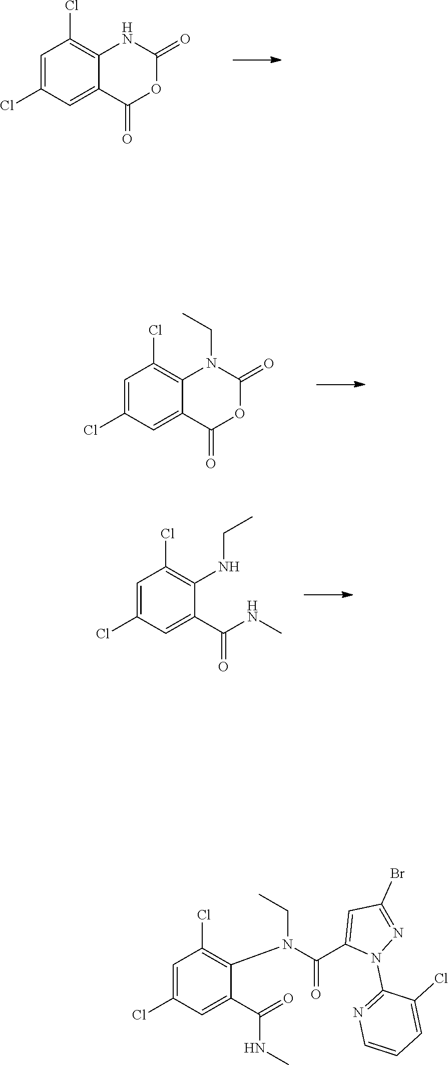 [SMILES: O=c1[nH]c2c(Cl)cc(Cl)cc2c(=O)o1 ; CCn1c(=O)oc(=O)c2cc(Cl)cc(Cl)c21 ; CCNc1c(Cl)cc(Cl)cc1C(=O)NC ; CCN(C(=O)c1cc(Br)nn1-c1ncccc1Cl)c1c(Cl)cc(Cl)cc1C(=O)NC]

### N-[2,4-dichloro-6-[(methylamino)carbonyl]phenyl]-N-ethyl-1-(3-chloro-2-pyridinyl)-3-bromo-1H-pyrazole-5-carboxamide

A mixture of N-methyl-3,5-dichloro-2-(ethylamino)benzamide (5.0 mmol) and 3-bromo-1-(3-chloro-2-pyridinyl)-1H-pyrazole-5-carbonyl chloride (5.5 mmol) in toluene (20 mL) or a mixture of toluene (5 mL) and xylene (15 mL) were refluxed for 2-6 hours with stirring until the reaction was complete. The reaction mixture was cooled to room temperature, the resulting crude products were collected by filtration and recrystallized from petroleum ether and ethyl acetate to afford the title compound in greater than 70% yield.

### Example 4 Preparation of Compound 10 in Table 1 and Compound II-10 in Table 3

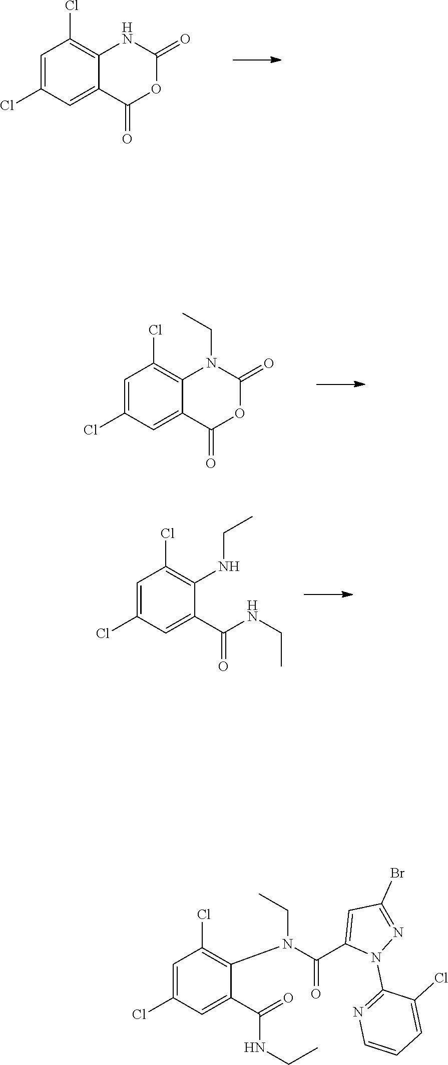 [SMILES: O=c1[nH]c2c(Cl)cc(Cl)cc2c(=O)o1 ; CCn1c(=O)oc(=O)c2cc(Cl)cc(Cl)c21 ; CCNC(=O)c1cc(Cl)cc(Cl)c1NCC ; CCNC(=O)c1cc(Cl)cc(Cl)c1N(CC)C(=O)c1cc(Br)nn1-c1ncccc1Cl]

### 6,8-dichloro-1-ethyl-2H-3,1-benzooxazine-2,4(1H)-dione

To a mixture of 6,8-dichloro-2H-3,1-benzooxazine-2,4(1H)-dione (0.02 mol) and sodium hydride (0.03 mol) in N,N-dimethylformamide (25 mL) was slowly added dropwise iodoethane (0.03 mol) under room temperature and stirring conditions. The reaction mixture was continued stirring 6-18 hours until the reaction was complete. The reaction mixture was poured into ice water and the resulting crude products were collected by filtration, washed with water and dried to afford the title compound in 80% yield.

### N-ethyl-3,5-dichloro-2-(ethylamino)benzamide

A mixture of 6,8-dichloro-1-ethyl-2H-3,1-benzooxazine-2,4(1H)-dione (0.01 mol) and ethylamine aqueous solution (65%, 10 mL) was stirred at room temperature for 2-6 hours until the reaction was complete. The reaction mixture was acidified with dilute hydrochloric acid to PH of about 7, the title compound in about 60% yield was afford by routine post-treatments such as the reaction mixture extracted with dichloromethane, washed with ice water, dried, concentrated and purified, etc.

### N-[2,4-dichloro-6-[(ethylamino)carbonyl]phenyl]-N-ethyl-1-(3-chloro-2-pyridinyl)-3-bromo-1H-pyrazole-5-carboxamide

A mixture of N-ethyl-3,5-dichloro-2-(ethylamino)benzamide (5.0 mmol) and 3-bromo-1-(3-chloro-2-pyridinyl)-1H-pyrazole-5-carbonyl chloride (5.5 mmol) in toluene (20 mL) or a mixture of toluene (5 mL) and xylene (15 mL) were refluxed for 2-6 hours with stirring until the reaction was complete. The reaction mixture was cooled to room temperature, the resulting crude products were collected by filtration and recrystallized from petroleum ether and ethyl acetate to afford the title compound in greater than 70% yield.

### Example 5 Preparation of Compound 11 in Table 1 and Compound II-11 in Table 3 (Method 1)

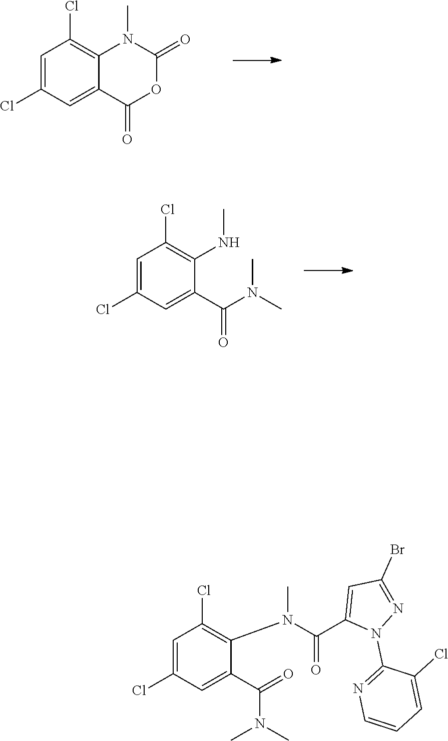 [SMILES: Cn1c(=O)oc(=O)c2cc(Cl)cc(Cl)c21 ; CNc1c(Cl)cc(Cl)cc1C(=O)N(C)C ; CN(C)C(=O)c1cc(Cl)cc(Cl)c1N(C)C(=O)c1cc(Br)nn1-c1ncccc1Cl]

### N,N-dimethyl-3,5-dichloro-2-(methylamino)benzamide

A mixture of 6,8-dichloro-1-methyl-2H-3,1-benzooxazine-2,4(1H)-dione (0.01 mol) and dimethylamine aqueous solution (30%, 10 mL) was stirred at room temperature for 2-6 hours until the reaction was complete. the title compound in about 70% yield was afford by routine post-treatments such as the reaction mixture extracted with ethyl acetate, washed with ice water, dried, concentrated and purified, etc.

### N-[2,4-dichloro-6-[(dimethylamino)carbonyl]phenyl]-N-methyl-1-(3-chloro-2-pyridinyl)-3-bromo-1H-pyrazole-5-carboxamide

A mixture of N,N-dimethyl-3,5-dichloro-2-(methylamino)benzamide (5.0 mmol) and 3-bromo-1-(3-chloro-2-pyridinyl)-1H-pyrazole-5-carbonyl chloride (5.5 mmol) in toluene (20 mL) or a mixture of toluene (5 mL) and xylene (15 mL) were refluxed for 2-6 hours with stirring until the reaction was complete. The reaction mixture was cooled to room temperature, the resulting crude products were collected by filtration and recrystallized from petroleum ether and ethyl acetate to afford the title compound in greater than 80% yield.

### Example 6 Preparation of Compound 11 in Table 1 (Method 2)

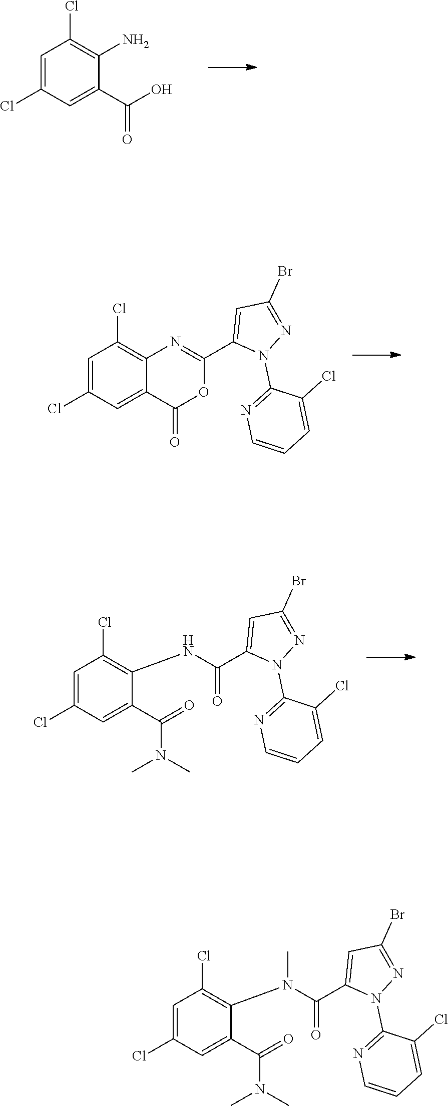 [SMILES: Nc1c(Cl)cc(Cl)cc1C(=O)O ; O=c1oc(-c2cc(Br)nn2-c2ncccc2Cl)nc2c(Cl)cc(Cl)cc12 ; CN(C)C(=O)c1cc(Cl)cc(Cl)c1NC(=O)c1cc(Br)nn1-c1ncccc1Cl ; CN(C)C(=O)c1cc(Cl)cc(Cl)c1N(C)C(=O)c1cc(Br)nn1-c1ncccc1Cl]

### 6,8-dichloro-2-[1-(3-chloro-2-pyridinyl)-3-bromo-1H-pyrazol-5-yl]-4H-3,1-benzooxazine-4-one

To a mixture of 2-amino-3,5-dichlorobenzoic acid (0.1 mol), 3-bromo-1-(3-chloro-2-pyridinyl)-1H-pyrazol-5-carboxylic acid (0.1 mol) and pyridine (0.4 mol) in acetonitrile (50 mL) was slowly added dropwise methanesulfonyl chloride (0.4 mol) under −10 to 0° C. and stirring conditions. The reaction mixture was stirred 3-5 hours at this temperature, then allowed to warm naturally to room temperature, and stirred 2-5 hours until the reaction was complete. The reaction mixture was added ice water, the resulting crude products were collected by filtration, washed with water, dried to afford the title compound in greater than 80% yield.

### N-[2,4-dichloro-6-[(dimethylamino)carbonyl]phenyl]-1-(3-chloro-2-pyridinyl)-3-bromo-1H-pyrazole-5-carboxamide

To a solution of 6,8-dichloro-2-[1-(3-chloro-2-pyridinyl)-3-bromo-1H-pyrazol-5-yl]-4H-3,1-benzooxazine-4-one (0.01 mol) in tetrahydrofuran (15 mL) was slowly added dropwise dimethylamine aqueous solution (33%, 15 mL) under −10 to 0° C. and stirring conditions. The reaction mixture was stirred 2-5 hours at this temperature, then allowed to warm naturally to room temperature, and stirred 2-3 hours until the reaction was complete. The reaction mixture was poured into ice water, the resulting title product in greater than 80% yield was collected by filtration.

### N-[2,4-dichloro-6-[(dimethylamino)carbonyl]phenyl]-N-methyl-1-(3-chloro-2-pyridinyl)-3-bromo-1H-pyrazole-5-carboxamide

To a solution of N-[2,4-dichloro-6-[(dimethylamino)carbonyl]phenyl]-1-(3-chloro-2-pyridinyl)-3-bromo-1H-pyrazole-5-carboxamide (0.015 mol) in N,N-dimethylformamide (30 mL) or a mixture of N,N-dimethylformamide (25 mL) and tetrahydrofuran (5 mL) was added in batches sodium hydride (0.025 mol) under cooling in an ice-water bath and stirring conditions. The mixture was stirred 15-30 minutes and then iodomethane (0.025 mol) was slowly added dropwise. The reaction mixture was stirred 1-3 hours at this temperature, then allowed to warm naturally to room temperature, and stirred 2-5 hours until the reaction was complete. The reaction mixture was poured slowly into saturated ice brine, the resulting crude products were collected by filtration and recrystallized from ethanol to afford the title compound in about 80% yield.

### Example 7 Preparation of Compound 15 in Table 1 and Compound II-15 in Table 3 (Method 1)

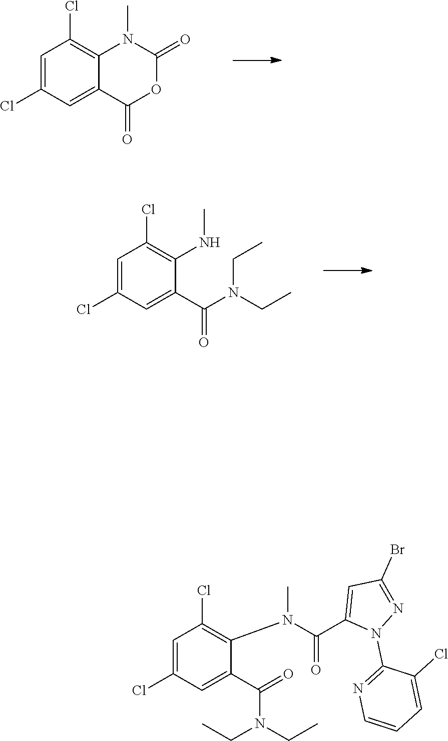 [SMILES: Cn1c(=O)oc(=O)c2cc(Cl)cc(Cl)c21 ; CCN(CC)C(=O)c1cc(Cl)cc(Cl)c1NC ; CCN(CC)C(=O)c1cc(Cl)cc(Cl)c1N(C)C(=O)c1cc(Br)nn1-c1ncccc1Cl]

### N,N-diethyl-3,5-dichloro-2-(methylamino)benzamide

A mixture of 6,8-dichloro-1-methyl-2H-3,1-benzooxazine-2,4(1H)-dione (0.01 mol), diethylamine (0.02 mol) and water (20 mL) was stirred at room temperature for 2-6 hours until the reaction was complete. the title compound in about 65% yield was afford by routine post-treatments such as the reaction mixture extracted with dichloromethane, washed with ice water, dried, concentrated and purified, etc.

### N-[2,4-dichloro-6-[(diethylamino)carbonyl]phenyl]-N-methyl-1-(3-chloro-2-pyridinyl)-3-bromo-1H-pyrazole-5-carboxamide

A mixture of N,N-diethyl-3,5-dichloro-2-(methylamino)benzamide (5.0 mmol) and 3-bromo-1-(3-chloro-2-pyridinyl)-1H-pyrazole-5-carbonyl chloride (5.5 mmol) in toluene (20 mL) or a mixture of toluene (5 mL) and xylene (15 mL) were refluxed for 2-6 hours with stirring until the reaction was complete. The reaction mixture was cooled to room temperature, the resulting crude products were collected by filtration and recrystallized from petroleum ether and ethyl acetate to afford the title compound in greater than 70% yield.

### Example 8 Preparation of Compound 15 in Table 1 (Method 2)

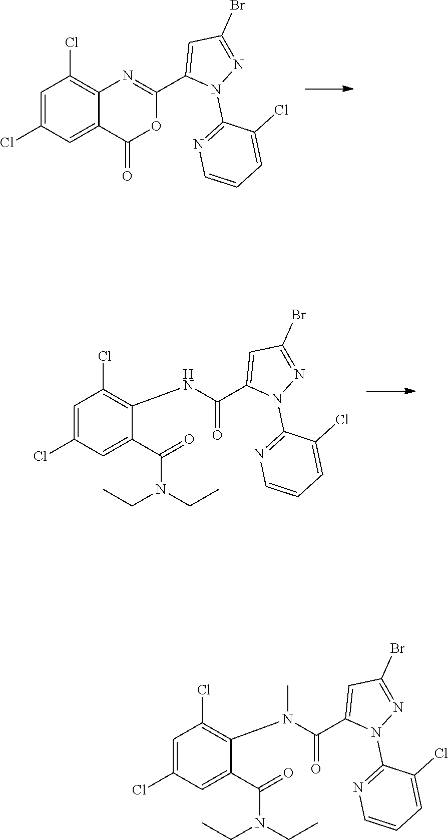 [SMILES: O=c1oc(-c2cc(Br)nn2-c2ncccc2Cl)nc2c(Cl)cc(Cl)cc12 ; CCN(CC)C(=O)c1cc(Cl)cc(Cl)c1NC(=O)c1cc(Br)nn1-c1ncccc1Cl ; CCN(CC)C(=O)c1cc(Cl)cc(Cl)c1N(C)C(=O)c1cc(Br)nn1-c1ncccc1Cl]

### N-[2,4-dichloro-6-[(diethylamino)carbonyl]phenyl]-1-(3-chloro-2-pyridinyl)-3-bromo-1H-pyrazole-5-carboxamide

To a mixture of 6,8-dichloro-2-[1-(3-chloro-2-pyridinyl)-3-bromo-1H-pyrazol-5-yl]-4H-3,1-benzooxazine-4-one (0.02 mol) in water (40 mL) was slowly added dropwise diethylamine (0.04 mol) under −10 to 0° C. and stirring conditions. The reaction mixture was stirred 2-5 hours at this temperature, then allowed to warm naturally to room temperature, and stirred 2-5 hours until the reaction was complete. The reaction mixture was added ice water, the title compound in about 70% yield was afford by routine post-treatments such as the reaction mixture extracted with dichloromethane, washed with ice water, dried, concentrated and purified, etc.

### N-[2,4-dichloro-6-[(diethylamino)carbonyl]phenyl]-N-methyl-1-(3-chloro-2-pyridinyl)-3-bromo-1H-pyrazole-5-carboxamide

To a solution of N-[2,4-dichloro-6-[(diethylamino)carbonyl]phenyl]-1-(3-chloro-2-pyridinyl)-3-bromo-1H-pyrazole-5-carboxamide (0.015 mol) in N,N-dimethylformamide (30 mL) or a mixture of N,N-dimethylformamide (25 mL) and tetrahydrofuran (5 mL) was added in batches sodium hydride (0.025 mol) under cooling in an ice-water bath and stirring conditions. The mixture was stirred 15-30 minutes and then iodomethane (0.025 mol) was slowly added dropwise. The reaction mixture was stirred 1-3 hours at this temperature, then allowed to warm naturally to room temperature, and stirred 2-5 hours until the reaction was complete. The reaction mixture was poured slowly into saturated ice brine, the resulting crude products were collected by filtration and recrystallized from ethanol to afford the title compound in greater than 70% yield.

Other compounds of this invention can be prepared by the procedures described in examples 1-8 or together with methods known in relevant references.

### BIOLOGICAL EXAMPLES OF THE INVENTION

The biological tests indicate that the compounds of this invention possess excellent insecticidal activity. Some results are as follows.

### Example 9 Test Against Armyworm (Mythimna separata (Walker))

Potter tower method: the test compound was dissolved in an appropriate solvent such as N,N-dimethylformamide, and then diluted with 0.2% aqueous solution of Tween 80 to the desired concentration, Those without the test compounds were set as blank controls. The fresh and tender corn leaves were taken and cut into fragments of basically the same size, then put into a Petri dish (diameter 90 mm) padded with filter paper in advance. Ten third-instar larvae of test insects were transferred into the dish and then placed under the Potter spray tower for being sprayed, each concentration treatment was sprayed at the amount of 1 mL and replicated 3 times. The dish was covered and then placed in a chamber for culture and observed regularly, after 72 hours, the dead insects was investigated and recorded and the mean mortality was calculated. Activity (mortality) is divided into four levels A, B, C and D as a percentage relative to the blank control, 100≥mortality (%)≥90 is level A, 90>mortality (%)≥70 is level B, 70>mortality (%)≥50 is level C, and 50>mortality (%)≥0 is level D. The results show that the compounds of this invention possess excellent activity against armyworm, and some compounds still have high activity at very low concentration. Some results are listed below:

At 500 mg/L, the following compounds of this invention as well as D1, D2 and D3 provided level A activity against armyworm: 1, 2, 3, 5, 6, 7, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, etc.

At 50 mg/L, the following compounds of this invention as well as D1, D2 and D3 provided level A activity against armyworm: 1, 2, 3, 5, 6, 7, 9, 10, 11, 12, 13, 14, 15, 16, 17, 18, 19, 20, 21, 22, 23, 24, 25, 26, 27, 28, 29, 32, 33, 34, 35, 36, 37, 38, 39, 40, 41, 42, 43, 44, 46, 47, 48, 49, etc.

At 12.5 mg/L, the following compounds of this invention as well as D1 and D2 provided level A activity and D3 provided level B activity against armyworm: 1, 2, 3, 5, 6, 10, 11, 12, 14, 15, 17, 22, 23, 25, 26, 28, 29, 32, 37, 40, 42, etc.

Further screening results showed that the compounds of this invention, such as 1, 2, 5, 9, 10, 11, 12, 14, 15, 32 and 40 etc., provided excellent activity against armyworm, and their activity against armyworm could rank with that of chlorantraniliprole (D1); The activity of some compounds of this invention against armyworm was better than that of chlorantraniliprole (D1). For example, at 1.25 mg/L, the activity of compound 1 of this invention against armyworm was 75%, and the activity of chlorantraniliprole (D1) against armyworm was less than 50% under the same conditions.

### Example 10 Test Against Spodoptera litura (Fabricius)

The test compound was dissolved in an appropriate solvent such as N,N-dimethylformamide, and then diluted with 0.2% aqueous solution of Tween 80 to the desired concentration, Those without the test compounds were set as blank controls, the treatments were replicated 3 times. The cabbage leaves were perforated to get 18 mm diameter leaf discs by a hole puncher, 5 leaf discs were dipped in the test solution for 10 seconds and then put into a Petri dish (diameter 90 mm) padded with moisturizing filter paper, after air-drying, the leaf discs were infested with no less than ten second-instar larvae of test insects which were starved for 4 hours, and then the dish was covered and placed in a chamber for culture. After 72 hours, the number of dead insects was investigated, the mortality was calculated, and the regression equation, correlation coefficient (r) and the median lethal concentration (LC₅₀) were obtained using Probit analysis method.

The results show that the compounds of this invention possess excellent activity against Spodoptera litura, and some compounds such as compounds 1, 2, 9, 10, 11, 12, 15, 32, etc. as well as chlorantraniliprole (D1) still have high activity at very low concentration. The activity against Spodoptera litura of some compounds of this invention is better than that of chlorantraniliprole (D1). For example, the median lethal concentration LC₅₀ value of compounds 1, 2, 10, 15, etc. of this invention against Spodoptera litura is lower than that of chlorantraniliprole (D1) under the same conditions.

### Example 11 Test Against Diamondback Moth (Plutella xylostella (Linnaeus))

The test compound was dissolved in an appropriate solvent such as N,N-dimethylformamide, and then diluted with 0.2% aqueous solution of Tween 80 to the desired concentration, Those without the test compounds were set as blank controls, the treatments were replicated 3 times. The cabbage leaves were perforated to get 18 mm diameter leaf discs by a hole puncher, 5 leaf discs were dipped in the test solution for 10 seconds and then put into a Petri dish (diameter 90 mm) padded with moisturizing filter paper, after air-drying, the leaf discs were infested with no less than ten second-instar larvae of test insects which were starved for 4 hours, and then the dish was covered and placed in a chamber for culture. After 72 hours, the number of dead insects was investigated, the mortality was calculated, and the regression equation, correlation coefficient (r) and the median lethal concentration (LC₅₀) were obtained using Probit analysis method.

The results show that the compounds of this invention possess excellent activity against diamondback moth, and some compounds such as compounds 1, 2, 9, 10, 11, 12, 15, 32, etc. as well as chlorantraniliprole (D1) still have high activity at very low concentration, for example, the lethal medium concentration LC₅₀ values of compounds 1, 2, 11 etc. of this invention against diamondback moth are lower than 1.00 mg/L. The activity against diamondback moth of some compounds of this invention is better than that of chlorantraniliprole (D1).

### Example 12 Test Against Aphid (Aphidoidea)

For evaluating the activity of the compounds of this invention against Homoptera pests, Aphis fabae, wheat aphids, etc. were selected as the test insects, and the activity of the compounds of this invention against aphids was measured by leaf dip method indoors.

Leaf dip method (Aphis fabae): the test compound was dissolved in an appropriate solvent such as N,N-dimethylformamide, and then diluted with 0.2% aqueous solution of Tween 80 to the desired concentration, Those without the test compounds were set as blank controls, the treatments were replicated 3 times. Horsebean seedlings with no less than twenty 3-day-old Aphis fabae per plant was cut from the stem, 5 horsebean seedlings with the insects were dipped in the test solution for 5 seconds, then inserted into a sponge filled with water after sucked dry using filter paper, then covered with a glass tube and placed in a chamber for culture. After 24 hours, the number of living and dead insects were investigated and the results were averaged. Activity (mortality) is divided into four levels A, B, C and D as a percentage relative to the blank control, 100≥mortality (%)≥90 is level A, 90>mortality (%)≥70 is level B, 70>mortality (%)≥50 is level C, and 50>mortality (%)≥0 is level D. The results show that the compounds of this invention possess excellent activity against Aphis fabae, and some compounds still have excellent activity at very low concentration. Some results are listed below:

At 500 mg/L, the following compounds of this invention as well as D1, D2 and D3 provided level A activity against Aphis fabae: 1, 2, 3, 5, 6, 7, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, etc.

At 200 mg/L, the following compounds of this invention as well as D2 and D3 provided level A activity against Aphis fabae: 1, 2, 3, 5, 6, 7, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 46, 47, 49, etc.

At 50 mg/L, the following compounds of this invention as well as D2 and D3 provided level A activity and D1 provided level D activity against Aphis fabae: 1, 2, 3, 5, 6, 7, 9, 10, 11, 12, 13, 14, 15, 16, 17, 18, 19, 21, 22, 25, 26, 27, 31, 32, 33, 34, 35, 36, 37, 38, 39, 40, 41, 42, 47, etc.

At 12.5 mg/L, the following compounds of this invention as well as D2 and D3 provided level A activity against Aphis fabae: 1, 2, 3, 5, 6, 9, 10, 11, 12, 13, 14, 15, 16, 17, 18, 19, 21, 22, 25, 26, 27, 31, 32, 33, 34, 35, 37, 38, 39, 40, 41, 42, 47, etc.

For further comparing the activity of the compounds of this invention with that of D2, D3 as well as the leading insecticide for controlling aphids imidacloprid (D4), compounds 1, 2, 5, 10, 11, 12, 15, 32, 33, 47, etc. selected as the representative compounds of this invention as well as D2, D3 and imidacloprid were further tested. The results show that the compounds of this invention possessed excellent activity against Aphis fabae, for example, the LC₅₀ values of compounds 1, 2, 10, 11, 15, 31, 34, etc. as well as imidacloprid against Aphis fabae were less than 1.0 mg/L, and the LC₅₀ values of D2 and D3 against Aphis fabae were greater than 1.0 mg/L.

Studies on the biological activity also show that the activity of some compounds of this invention against Aphis fabae is better than that of imidacloprid (D4) under the same conditions.

Studies on the biological activity against wheat aphids also show that the compounds of this invention possess excellent insecticidal activity against wheat aphids.

1. A 1-pyridyl-pyrazolyl amide compound, wherein the 1-pyridyl-pyrazolyl amide compound and its isomer thereof are represented by general Formula (I): 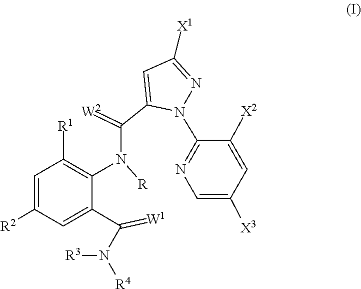
wherein:
I. R is C₁-C₁₂ alkyl, C₂-C₁₂ alkenyl, C₂-C₁₂ alkynyl, C₃-C₈ cycloalkyl or C₃-C₈ heterocyclic group;
II. R¹ is halogen, C₁-C₆ alkyl or C₁-C₆ alkoxy;
III. R² is halogen, cyano, C₁-C₆ alkyl, C₁-C₆ alkoxy or nitro;
IV. R³ is H, C₁-C₁₂ alkyl, C₂-C₁₂ alkenyl, C₂-C₁₂ alkynyl, C₃-C₈ cycloalkyl or C₃-C₈ heterocyclic group;
V. R⁴ is C₁-C₁₂ alkyl, C₂-C₁₂ alkenyl, C₂-C₁₂ alkynyl, C₃-C₈ cycloalkyl or C₃-C₈ heterocyclic group;
VI. X¹, X² and X³ are identical or different, and they represent H, halogen, C₁-C₆ alkyl or C₁-C₆ alkoxy;
VII. W¹ and W² are identical or different, and they represent oxygen or sulfur;
in above definition of Formula (I), the terms used whether alone or in compound words, represent the following substituents:
halogen: represents fluorine, chlorine, bromine or iodine;
alkyl: represents straight-chain or branched-chain alkyl;
cycloalkyl: represents saturated or unsaturated cycloalkyl;
alkenyl: represents straight-chain or branched-chain alkenyl;
alkynyl: represents straight-chain or branched-chain alkynyl;
halo-: represents the hydrogen atoms are partially or entirely substituted by halogen atoms;
heterocyclic group: represents saturated or unsaturated heterocyclic group containing one or more than one identical or different hetero-atoms selected from N, O and/or S, including furanyl, thiophenyl, pyrrolyl, piperidinyl, piperazinyl, pyrazinyl, pyridazinyl, oxazolyl, thiazolyl, pyrazolyl, imidazolyl, tetrazolyl or triazolyl.

2. The 1-pyridyl-pyrazolyl amide compound according to claim 1, wherein
I. R is C₁-C₁₂ alkyl or C₃-C₅ cycloalkyl;
II. R¹ is halogen or CH₃;
III. R² is halogen, CF₃, nitro or CN;
IV. R³ is H, C₁-C₁₂ alkyl or C₃-C₅ cycloalkyl;
V. R⁴ is C₁-C₁₂ alkyl or C₃-C₅ cycloalkyl;
VI. X¹, X² and X³ are identical or different, and they represent H, halogen or CF₃;
VII. W¹ and W² are identical or different, and they represent oxygen or sulfur.

3. The 1-pyridyl-pyrazolyl amide compounds according to claim 1, wherein the compound represented by Formula (I) is:
N-[2,4-dichloro-6-[(methylamino)carbonyl]phenyl]-N-methyl-1-(3-chloro-2-pyridinyl)-3-bromo-1H-pyrazole-5-carboxamide;
N-[2,4-dichloro-6-[(ethylamino)carbonyl]phenyl]-N-methyl-1-(3-chloro-2-pyridinyl)-3-bromo-1H-pyrazole-5-carboxamide;
N-[2,4-dichloro-6-[(propylamino)carbonyl]phenyl]-N-methyl-1-(3-chloro-2-pyridinyl)-3-bromo-1H-pyrazole-5-carboxamide;
N-[2,4-dichloro-6-[(isopropylamino)carbonyl]phenyl]-N-methyl-1-(3-chloro-2-pyridinyl)-3-bromo-1H-pyrazole-5-carboxamide;
N-[2,4-dichloro-6-[(cyclopropylamino)carbonyl]phenyl]-N-methyl-1-(3-chloro-2-pyridinyl)-3-bromo-1H-pyrazole-5-carboxamide;
N-[2,4-dichloro-6-[(butylamino)carbonyl]phenyl]-N-methyl-1-(3-chloro-2-pyridinyl)-3-bromo-1H-pyrazole-5-carboxamide;
N-[2,4-dichloro-6-[(tert-butylamino)carbonyl]phenyl]-N-methyl-1-(3-chloro-2-pyridinyl)-3-bromo-1H-pyrazole-5-carboxamide;
N-[2,4-dichloro-6-[(methylamino)carbonyl]phenyl]-N-ethyl-1-(3-chloro-2-pyridinyl)-3-bromo-1H-pyrazole-5-carboxamide;
N-[2,4-dichloro-6-[(ethylamino)carbonyl]phenyl]-N-ethyl-1-(3-chloro-2-pyridinyl)-3-bromo-1H-pyrazole-5-carboxamide;
N-[2,4-dichloro-6-[(dimethylamino)carbonyl]phenyl]-N-methyl-1-(3-chloro-2-pyridinyl)-3-bromo-1H-pyrazole-5-carboxamide;
N-[2,4-dichloro-6-[(methyl(ethyl)amino)carbonyl]phenyl]-N-methyl-1-(3-chloro-2-pyridinyl)-3-bromo-1H-pyrazole-5-carboxamide;
N-[2,4-dichloro-6-[(methyl(isopropyl)amino)carbonyl]phenyl]-N-methyl-1-(3-chloro-2-pyridinyl)-3-bromo-1H-pyrazole-5-carboxamide
N-[2,4-dichloro-6-[(methyl(cyclopropyl)amino)carbonyl]phenyl]-N-methyl-1-(3-chloro-2-pyridinyl)-3-bromo-1H-pyrazole-5-carboxamide;
N-[2,4-dichloro-6-[(diethylamino)carbonyl]phenyl]-N-methyl-1-(3-chloro-2-pyridinyl)-3-bromo-1H-pyrazole-5-carboxamide;
N-[2,4-dichloro-6-[(dipropylamino)carbonyl]phenyl]-N-methyl-1-(3-chloro-2-pyridinyl)-3-bromo-1H-pyrazole-5-carboxamide;
N-[2,4-dichloro-6-[(dimethylamino)carbonyl]phenyl]-N-ethyl-1-(3-chloro-2-pyridinyl)-3-bromo-1H-pyrazole-5-carboxamide;
N-[2,4-dichloro-6-[(methyl(ethyl)amino)carbonyl]phenyl]-N-ethyl-1-(3-chloro-2-pyridinyl)-3-bromo-1H-pyrazole-5-carboxamide;
N-[2,4-dichloro-6-[(dimethylamino)carbonyl]phenyl]-N-propyl-1-(3-chloro-2-pyridinyl)-3-bromo-1H-pyrazole-5-carboxamide;
N-[2,4-dichloro-6-[(methyl(propyl)amino)carbonyl]phenyl]-N-propyl-1-(3-chloro-2-pyridinyl)-3-bromo-1H-pyrazole-5-carboxamide;
N-[2,4-dichloro-6-[(dimethylamino)thioxomethyl]phenyl]-N-methyl-1-(3-chloro-2-pyridinyl)-3-bromo-1H-pyrazole-5-carboxamide;
N-[2,4-dichloro-6-[(methyl(isopropyl)amino)thioxomethyl]phenyl]-N-methyl-1-(3-chloro-2-pyridinyl)-3-bromo-1H-pyrazole-5-carboxamide;
N-[2,4-dichloro-6-[(diethylamino)thioxomethyl]phenyl]-N-methyl-1-(3-chloro-2-pyridinyl)-3-bromo-1H-pyrazole-5-carboxamide;
N-[2,4-dichloro-6-[(diethylamino)thioxomethyl]phenyl]-N-methyl-1-(3-chloro-2-pyridinyl)-3-bromo-1H-pyrazole-5-carbothioamide;
N-[2-bromo-4-chloro-6-[(methylamino)carbonyl]phenyl]-N-methyl-1-(3-chloro-2-pyridinyl)-3-bromo-1H-pyrazole-5-carboxamide;
N-[2-bromo-4-chloro-6-[(ethylamino)carbonyl]phenyl]-N-methyl-1-(3-chloro-2-pyridinyl)-3-bromo-1H-pyrazole-5-carboxamide;
N-[2-bromo-4-chloro-6-[(propylamino)carbonyl]phenyl]-N-methyl-1-(3-chloro-2-pyridinyl)-3-bromo-1H-pyrazole-5-carboxamide;
N-[2-bromo-4-chloro-6-[(isopropylamino)carbonyl]phenyl]-N-methyl-1-(3-chloro-2-pyridinyl)-1H-pyrazole-5-carboxamide;
N-[2-bromo-4-chloro-6-[(isopropylamino)carbonyl]phenyl]-N-methyl-1-(3-chloro-2-pyridinyl)-3-bromo-1H-pyrazole-5-carboxamide;
N-[2-bromo-4-chloro-6-[(cyclopropylamino)carbonyl]phenyl]-N-methyl-1-(3-chloro-2-pyridinyl)-3-bromo-1H-pyrazole-5-carboxamide;
N-[2-bromo-4-chloro-6-[(butylamino)carbonyl]phenyl]-N-methyl-1-(3-chloro-2-pyridinyl)-1H-pyrazole-5-carboxamide;
N-[2-bromo-4-chloro-6-[(butylamino)carbonyl]phenyl]-N-methyl-1-(3-chloro-2-pyridinyl)-3-bromo-1H-pyrazole-5-carboxamide;
N-[2-bromo-4-chloro-6-[(dimethylamino)carbonyl]phenyl]-N-methyl-1-(3-chloro-2-pyridinyl)-3-bromo-1H-pyrazole-5-carboxamide;
N-[2-bromo-4-chloro-6-[(methyl(ethyl)amino)carbonyl]phenyl]-N-methyl-1-(3-chloro-2-pyridinyl)-3-bromo-1H-pyrazole-5-carboxamide;
N-[2-bromo-4-chloro-6-[(methyl(propyl)amino)carbonyl]phenyl]-N-methyl-1-(3-chloro-2-pyridinyl)-3-bromo-1H-pyrazole-5-carboxamide;
N-[2-bromo-4-chloro-6-[(methyl(isopropyl)amino)carbonyl]phenyl]-N-methyl-1-(3-chloro-2-pyridinyl)-3-bromo-1H-pyrazole-5-carboxamide;
N-[2-bromo-4-chloro-6-[(methyl(cyclopropyl)amino)carbonyl]phenyl]-N-methyl-1-(3-chloro-2-pyridinyl)-3-bromo-1H-pyrazole-5-carboxamide;
N-[2-bromo-4-chloro-6-[(methyl(tert-butyl)amino)carbonyl]phenyl]-N-methyl-1-(3-chloro-2-pyridinyl)-3-bromo-1H-pyrazole-5-carboxamide;
N-[2-bromo-4-chloro-6-[(diethylamino)carbonyl]phenyl]-N-methyl-1-(3-chloro-2-pyridinyl)-3-bromo-1H-pyrazole-5-carboxamide;
N-[2-bromo-4-chloro-6-[(dimethylamino)carbonyl]phenyl]-N-ethyl-1-(3-chloro-2-pyridinyl)-3-bromo-1H-pyrazole-5-carboxamide;
N-[2-bromo-4-chloro-6-[(methyl(ethyl)amino)carbonyl]phenyl]-N-ethyl-1-(3-chloro-2-pyridinyl)-3-bromo-1H-pyrazole-5-carboxamide;
N-[2-bromo-4-chloro-6-[(diethylamino)carbonyl]phenyl]-N-ethyl-1-(3-chloro-2-pyridinyl)-3-bromo-1H-pyrazole-5-carboxamide;
N-[2-bromo-4-chloro-6-[(dimethylamino)carbonyl]phenyl]-N-propyl-1-(3-chloro-2-pyridinyl)-3-bromo-1H-pyrazole-5-carboxamide;
N-[2-methyl-4-chloro-6-[(methylamino)carbonyl]phenyl]-N-methyl-1-(3-chloro-2-pyridinyl)-3-bromo-1H-pyrazole-5-carboxamide;
N-[2-methyl-4-chloro-6-[(ethylamino)carbonyl]phenyl]-N-methyl-1-(3-chloro-2-pyridinyl)-3-bromo-1H-pyrazole-5-carboxamide;
N-[2-methyl-4-chloro-6-[(dimethylamino)carbonyl]phenyl]-N-methyl-1-(3-chloro-2-pyridinyl)-3-bromo-1H-pyrazole-5-carboxamide;
N-[2-methyl-4-chloro-6-[(methyl(ethyl)amino)carbonyl]phenyl]-N-methyl-1-(3-chloro-2-pyridinyl)-3-bromo-1H-pyrazole-5-carboxamide;
N-[2-methyl-4-chloro-6-[(methyl(propyl)amino)carbonyl]phenyl]-N-methyl-1-(3-chloro-2-pyridinyl)-3-bromo-1H-pyrazole-5-carboxamide;
N-[2-methyl-4-chloro-6-[(dimethylamino)thioxomethyl]phenyl]-N-methyl-1-(3-chloro-2-pyridinyl)-3-bromo-1H-pyrazole-5-carboxamide;
N-[2-methyl-4-bromo-6-[(diethylamino)carbonyl]phenyl]-N-methyl-1-(3-chloro-2-pyridinyl)-3-bromo-1H-pyrazole-5-carboxamide;
N-[2-methyl-4-bromo-6-[(diethylamino)carbonyl]phenyl]-N-ethyl-1-(3-chloro-2-pyridinyl)-3-bromo-1H-pyrazole-5-carboxamide.

4. A compound of formula (II) as a synthetic intermediate for preparing the 1-pyridyl-pyrazolyl amide compound represented by Formula (I) of claim 1, 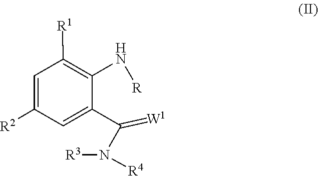
wherein:
R is C₁-C₁₂ alkyl, C₂-C₁₂ alkenyl, C₂-C₁₂ alkynyl, C₃-C₈ cycloalkyl or C₃-C₈ heterocyclic group;
R¹ is halogen, C₁-C₆ alkyl or C₁-C₆ alkoxy;
R² is halogen, cyano, C₁-C₆ alkyl, C₁-C₆ alkoxy or nitro;
R³ is H, C₁-C₁₂ alkyl, C₂-C₁₂ alkenyl, C₂-C₁₂ alkynyl, C₃-C₈ cycloalkyl or C₃-C₈ heterocyclic group;
R⁴ is C₁-C₁₂ alkyl, C₂-C₁₂ alkenyl, C₂-C₁₂ alkynyl, C₃-C₈ cycloalkyl or C₃-C₈ heterocyclic group;
W¹ represents oxygen or sulfur;
in above definition of Formula (II), the terms used whether alone or in compound words, represent the following substituents:
halogen: represents fluorine, chlorine, bromine or iodine;
alkyl: represents straight-chain or branched-chain alkyl;
cycloalkyl: represents saturated or unsaturated cycloalkyl;
alkenyl: represents straight-chain or branched-chain alkenyl;
alkynyl: represents straight-chain or branched-chain alkynyl;
halo-: represents the hydrogen atoms are partially or entirely substituted by halogen atoms;
heterocyclic group: represents saturated or unsaturated heterocyclic group containing one or more than one identical or different hetero-atoms selected from N, O and/or S, including furanyl, thiophenyl, pyrrolyl, piperidinyl, piperazinyl, pyrazinyl, pyridazinyl, oxazolyl, thiazolyl, pyrazolyl, imidazolyl, tetrazolyl or triazolyl.

5. A compound of formula (II) as a synthetic intermediate for preparing the 1-pyridyl-pyrazolyl amide compound represented by Formula (I) of claim 1, 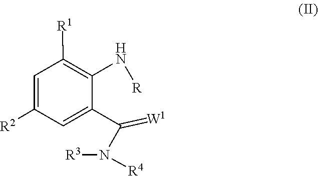
wherein the compound of formula (II) represents:
N-methyl-3,5-dichloro-2-(methylamino)benzamide;
N-ethyl-3,5-dichloro-2-(methylamino)benzamide;
N-propyl-3,5-dichloro-2-(methylamino)benzamide;
N-isopropyl-3,5-dichloro-2-(methylamino)benzamide;
N-cyclopropyl-3,5-dichloro-2-(methylamino)benzamide;
N-butyl-3,5-dichloro-2-(methylamino)benzamide;
N-methyl-3,5-dichloro-2-(ethylamino)benzamide;
N-ethyl-3,5-dichloro-2-(ethylamino)benzamide;
N,N-dimethyl-3,5-dichloro-2-(methylamino)benzamide;
N-methyl-N-ethyl-3,5-dichloro-2-(methylamino)benzamide;
N-methyl-N-isopropyl-3,5-dichloro-2-(methylamino)benzamide;
N-methyl-N-cyclopropyl-3,5-dichloro-2-(methylamino)benzamide;
N,N-diethyl-3,5-dichloro-2-(methylamino)benzamide;
N,N-dipropyl-3,5-dichloro-2-(methylamino)benzamide;
N,N-dimethyl-3,5-dichloro-2-(ethylamino)benzamide;
N-methyl-N-ethyl-3,5-dichloro-2-(ethylamino)benzamide;
N,N-dimethyl-3,5-dichloro-2-(propylamino)benzamide;
N-methyl-N-propyl-3,5-dichloro-2-(propylamino)benzamide;
N,N-dimethyl-3,5-dichloro-2-(methylamino)benzothioamide;
N-methyl-N-isopropyl-3,5-dichloro-2-(methylamino)benzothioamide;
N,N-diethyl-3,5-dichloro-2-(methylamino)benzothioamide;
N-methyl-3-bromo-5-chloro-2-(methylamino)benzamide;
N-ethyl-3-bromo-5-chloro-2-(methylamino)benzamide;
N-propyl-3-bromo-5-chloro-2-(methylamino)benzamide;
N-isopropyl-3-bromo-5-chloro-2-(methylamino)benzamide;
N-cyclopropyl-3-bromo-5-chloro-2-(methylamino)benzamide;
N-butyl-3-bromo-5-chloro-2-(methylamino)benzamide;
N,N-dimethyl-3-bromo-5-chloro-2-(methylamino)benzamide;
N-methyl-N-ethyl-3-bromo-5-chloro-2-(methylamino)benzamide;
N-methyl-N-propyl-3-bromo-5-chloro-2-(methylamino)benzamide;
N-methyl-N-isopropyl-3-bromo-5-chloro-2-(methylamino)benzamide;
N-methyl-N-cyclopropyl-3-bromo-5-chloro-2-(methylamino)benzamide;
N-methyl-N-tert-butyl-3-bromo-5-chloro-2-(methylamino)benzamide;
N,N-diethyl-3-bromo-5-chloro-2-(methylamino)benzamide;
N,N-dimethyl-3-bromo-5-chloro-2-(ethylamino)benzamide;
N-methyl-N-ethyl-3-bromo-5-chloro-2-(ethylamino)benzamide;
N,N-diethyl-3-bromo-5-chloro-2-(ethylamino)benzamide;
N,N-dimethyl-3-bromo-5-chloro-2-(propylamino)benzamide;
N-methyl-3-methyl-5-chloro-2-(methylamino)benzamide;
N-ethyl-3-methyl-5-chloro-2-(methylamino)benzamide;
N,N-dimethyl-3-methyl-5-chloro-2-(methylamino)benzamide;
N-methyl-N-ethyl-3-methyl-5-chloro-2-(methylamino)benzamide;
N,N-dimethyl-3-methyl-5-chloro-2-(methylamino)benzothioamide;
N,N-diethyl-3-methyl-5-bromo-2-(methylamino)benzamide
N,N-diethyl-3-methyl-5-bromo-2-(ethylamino)benzamide;
N-methyl-N-propyl-3-methyl-5-chloro-2-(methylamino)benzamide.

6. The 1-pyridyl-pyrazolyl amide compound according to claim 1, wherein the 1-pyridyl-pyrazolyl amide compound represented by Formula (I) exists as one or more isomers, the isomers include enantiomers, diastereomers, geometric isomers, rotational isomers and tautomers;
the 1-pyridyl-pyrazolyl amide compound represented by Formula (I) exists as geometric isomers, and the geometric isomer is represented by Z-isomer or E-isomer, the compound includes the Z-isomer, the E-isomer or their mixture in any proportion;
the 1-pyridyl-pyrazolyl amide compound represented by Formula (I) exists as stereoisomers, and the stereoisomer is represented by R-isomer or S-isomer, the compound includes the R-isomer, the S-isomer or their mixture in any proportion;
the 1-pyridyl-pyrazolyl amide compound represented by Formula (I) exists as rotational isomers, and the rotational isomer is represented by I-isomer or I′-isomer, the compound includes the I-isomer, the I′-isomer or their mixture in any proportion; or
the 1-pyridyl-pyrazolyl amide compound represented by Formula (I) exists as keto-enol tautomers, and the keto-enol tautomer is represented by I-isomer or I″-isomer, the compound includes the I-isomer, the I″ isomers or their mixture in any proportion.

7. The 1-pyridyl pyrazole amide compound according to claim 1, wherein a method for preparing the 1-pyridyl-pyrazolyl amide compound represented by Formula (I) comprises the following steps: 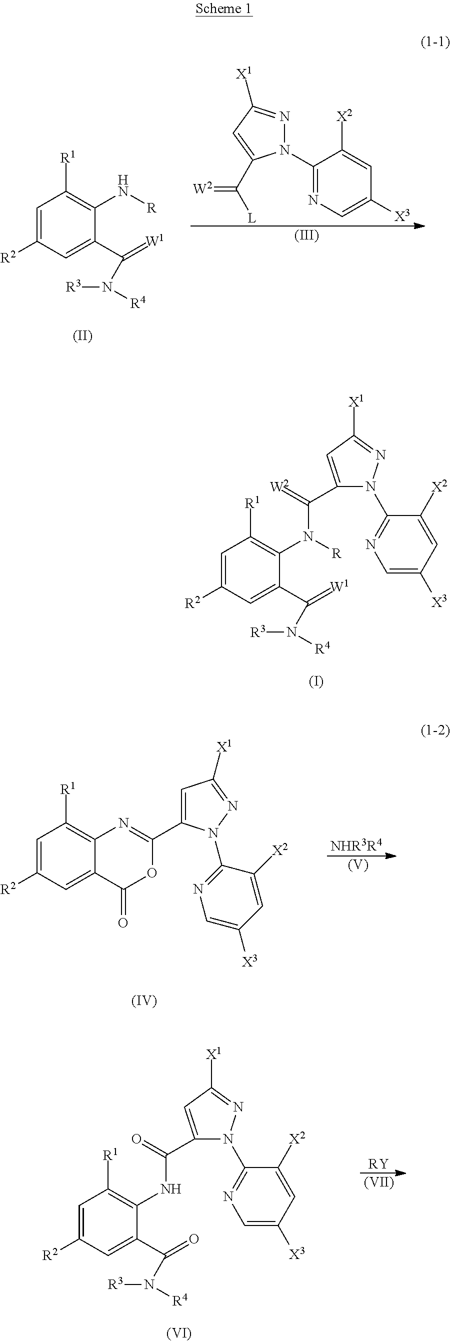 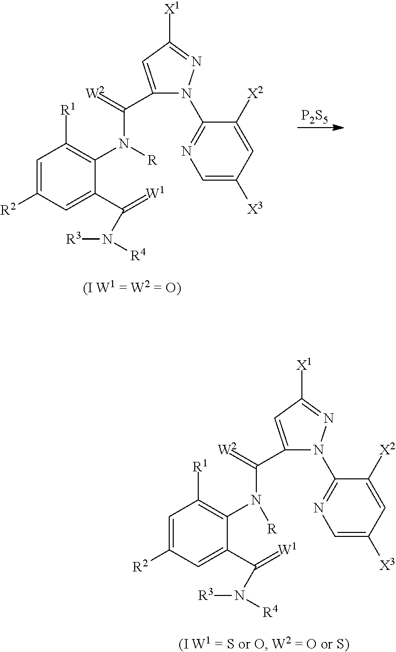 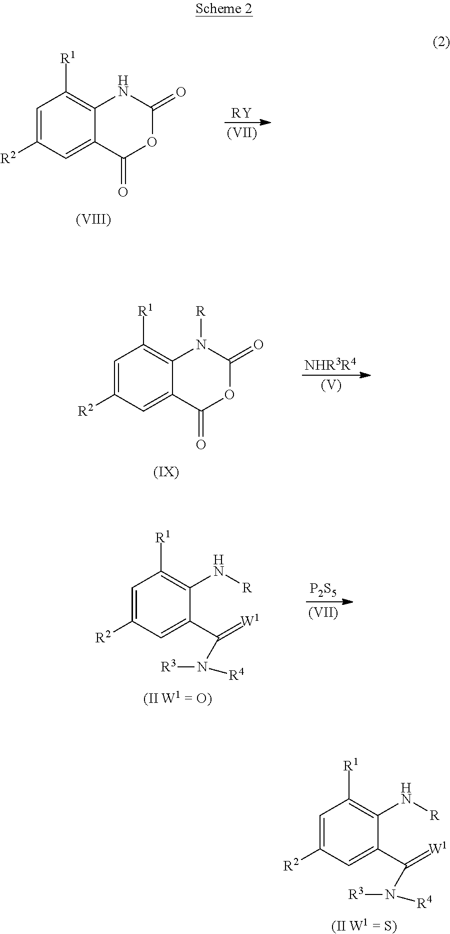 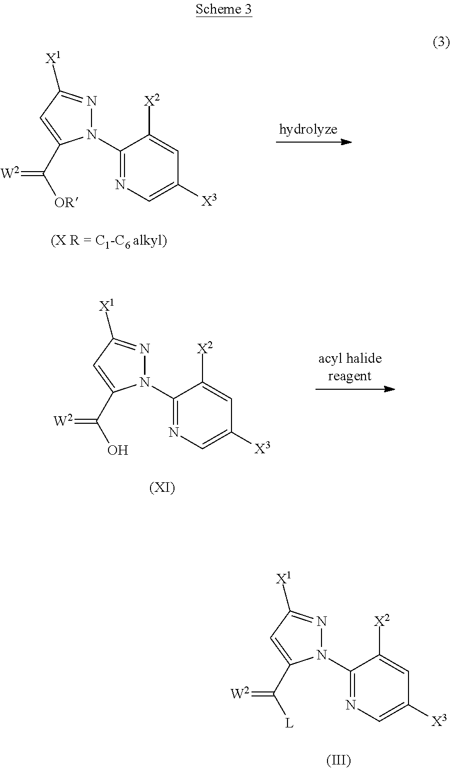 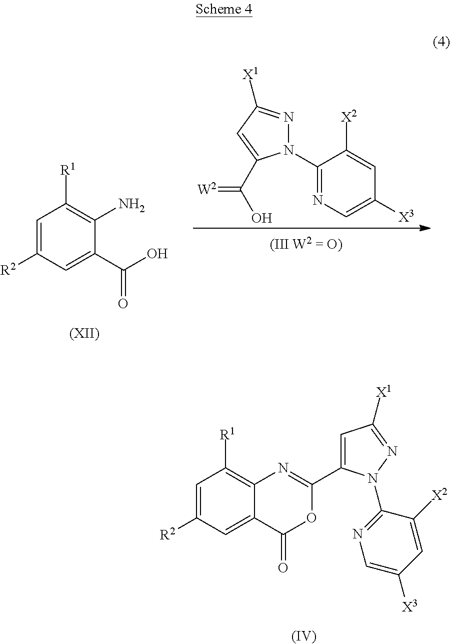 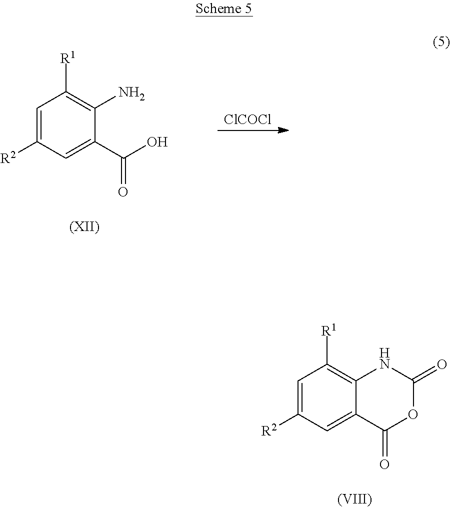
in a solvent or a mixture of any two solvents, at certain temperatures from −10° C. to the reflux temperature of the solvent used, reacting the compound of Formula (II) with a compound of Formula (III) to afford the compound of Formula (I), the addition of a base accelerates or facilitates the reaction, the solvent is selected from tetrahydrofuran, 1,4-dioxane, acetone, benzene, toluene, xylene, dichloromethane, dichloroethane, chloroform, tetrachloromethane, hexane, ethyl acetate, acetonitrile, dimethyl sulfoxide or N,N-dimethylformamide, the base is selected from triethylamine, N,N-dimethylaniline, pyridine, sodium hydroxide, potassium hydroxide, sodium carbonate, potassium carbonate, sodium hydride, sodium methoxide, sodium tert-butoxide, potassium tert-butoxide or sodium bicarbonate;
in a solvent or a mixture of any two solvents, at certain temperatures from −15° C. to the reflux temperature of the solvent used, reacting a compound of Formula (IV) with a compound of Formula (V) to afford a compound of Formula (VI), the solvent is selected from water, tetrahydrofuran, 1,4-dioxane, acetone, benzene, toluene, xylene, dichloromethane, dichloroethane, chloroform, tetrachloromethane, hexane, ethyl acetate, acetonitrile, dimethyl sulfoxide or N,N-dimethylformamide; in a solvent or a mixture of any two solvents, at certain temperatures from −15° C. to the reflux temperature of the solvent used, in the presence of a base, reacting the compound of Formula (VI) with a compound of Formula (VII) to afford a compound of Formula (I W¹=W²=0), the solvent is selected from tetrahydrofuran, 1,4-dioxane, acetone, benzene, toluene, xylene, dichloromethane, dichloroethane, chloroform, tetrachloromethane, hexane, ethyl acetate, acetonitrile, dimethyl sulfoxide or N,N-dimethylformamide, the base is selected from triethylamine, N,N-dimethylaniline, pyridine, methylpyridine, sodium hydroxide, potassium hydroxide, sodium carbonate, potassium carbonate, sodium hydride, sodium methoxide, sodium tert-butoxide, potassium tert-butoxide or sodium bicarbonate; in a solvent or a mixture of any two solvents, at certain temperatures from −15° C. to the reflux temperature of the solvent used, reacting a compound of Formula (I W¹=W²=O) with P₂S₅ to afford a compound of Formula (I W¹=S or O, W²=O or S), the solvent is selected from tetrahydrofuran, 1,4-dioxane, acetone, benzene, toluene, xylene, dichloromethane, dichloroethane, chloroform, tetrachloromethane, hexane, ethyl acetate, acetonitrile, dimethyl sulfoxide or N,N-dimethylformamide;
in a solvent or a mixture of any two solvents, at certain temperatures from −15° C. to the reflux temperature of the solvent used, in the presence of a base, reacting a compound of Formula (VIII) with the compound of Formula (VII) to afford the compound of Formula (IX), the solvent is selected from tetrahydrofuran, 1,4-dioxane, acetone, benzene, toluene, xylene, dichloromethane, dichloroethane, chloroform, tetrachloromethane, hexane, ethyl acetate, acetonitrile, dimethyl sulfoxide or N,N-dimethylformamide, the base is selected from triethylamine, N,N-dimethylaniline, pyridine, methylpyridine, sodium hydroxide, potassium hydroxide, sodium carbonate, potassium carbonate, sodium hydride, sodium methoxide, sodium tert-butoxide, potassium tert-butoxide or sodium bicarbonate; in a solvent or a mixture of any two solvents, at certain temperatures from −15° C. to the reflux temperature of the solvent used, in the presence of a base, reacting the compound of Formula (IX) with the compound of Formula (V) to afford a compound of Formula (II W¹=O), the solvent is selected from water, tetrahydrofuran, 1,4-dioxane, benzene, toluene, xylene, dichloromethane, dichloroethane, chloroform, tetrachloromethane, hexane, ethyl acetate, acetonitrile, dimethyl sulfoxide or N,N-dimethylformamide; in a solvent or a mixture of any two solvents, at certain temperatures from −15° C. to the reflux temperature of the solvent used, reacting the compound of Formula (II W¹=O) with P₂S₅ to afford a compound of Formula (II W¹=S), the solvent is selected from tetrahydrofuran, 1,4-dioxane, benzene, toluene, xylene, dichloromethane, dichloroethane, chloroform, tetrachloromethane, hexane, ethyl acetate, acetonitrile, dimethyl sulfoxide or N,N-dimethylformamide;
in a solvent or a mixture of any two solvents, at certain temperatures from 25° C. to the reflux temperature of the solvent used, in the presence of a base, routine hydrolysis of a compound of Formula (X) and then treating the reaction mixture with dilute acid solution such as dilute hydrochloric acid solution to afford a compound of Formula (XI), the solvent is selected from water, ethanol, methanol, tetrahydrofuran, 1,4-dioxane, benzene, toluene, xylene, dichloromethane, dichloroethane, chloroform, tetrachloromethane, hexane, ethyl acetate, acetonitrile, dimethyl sulfoxide or N,N-dimethylformamide, the base is selected from triethylamine, N,N-dimethylaniline, pyridine, methylpyridine, sodium hydroxide, potassium hydroxide, sodium carbonate, potassium carbonate, sodium hydride, sodium methoxide, sodium tert-butoxide, potassium tert-butoxide or sodium bicarbonate; in solvent-free or a solvent, at certain temperatures from 15° C. to the reflux temperature of the solvent used, reacting the compound of Formula (XI) with an acyl halide reagent to afford the compound of Formula (III), the solvent is selected from tetrahydrofuran, 1,4-dioxane, benzene, toluene, xylene, dichloromethane, dichloroethane, chloroform, tetrachloromethane, hexane, ethyl acetate or acetonitrile, the acyl halide reagent is selected from sulfoxide chloride, phosgene, phosphorus trichloride, phosphorus tribromide, phosphorus pentachloride, phosphorus pentabromide or oxalyl chloride;
in a solvent or a mixture of any two solvents, at certain temperatures from −15° C. to the reflux temperature of the solvent used, in the presence of a base, treating a mixture of a compound of Formula (XII) and a compound of Formula (III W²=O) with methylsulfonyl chloride to afford the compound of Formula (IV), the solvent is selected from tetrahydrofuran, 1,4-dioxane, acetone, toluene, xylene, acetonitrile, dichloromethane, dichloroethane or N,N-dimethylformamide, the base is pyridine or methylpyridine;
in a solvent or a mixture of any two solvents, at −15° C. to 25° C., reacting the compound of Formula (XII) with phosgene, diphosgene or triphosgene for 2 to 10 hours, and then at certain temperatures from 15° C. to the reflux temperature for 2 to 10 hours, to afford the compound of Formula (VIII), the solvent is selected from tetrahydrofuran, acetone, 1,4-dioxane, benzene, toluene, xylene, dichloromethane, dichloroethane, chloroform, tetrachloromethane, hexane, ethyl acetate or acetonitrile;
wherein R, R¹, R², R³, R⁴, X¹, X², X³, W¹ and W² are the same as defined in claim 1, L is a leaving group including chlorine or bromine.

8. An use of the 1-pyridyl pyrazole amide compound according to claim 1, wherein the compound possesses insecticidal activity comprising contacting the pests or their environment with the compound of Formula (I) at a dosage of 15 to 5000 g per hectare.

9. (canceled)

10. An insecticidal composition, comprising a biologically effective amount of a compound according to claim 1 and at least one additional component selected from the group consisting of surfactants, solid diluents and liquid diluents, in which the weight percentage of the active ingredient is in the range of 0.5-99%.

11. The 1-pyridyl-pyrazolyl amide compound according to claim 1, wherein: the hydrogen atoms in alkyl, alkenyl, alkynyl, cycloalkyl or heterocyclic group defined in I, II, III, IV, V and VI are partially or entirely substituted by identical or different substituting groups, selected from the following: halogen, cyano, hydroxyl, amino, mercapto, C₁-C₆ alkyl, C₁-C₆ alkoxy, C₁-C₆ alkylcarbonyl, C₁-C₆ alkoxycarbonyl, C₁-C₆ alkylthio, C₁-C₆ alkylamino, C₁-C₆ alkylsulfinyl, C₁-C₆ alkylsulfonyl or C₃-C₈ heterocyclic group.

12. The compound of formula (II) as the synthetic intermediate for preparing the 1-pyridyl-pyrazolyl amide compound represented by Formula (I) of claim 4, wherein:
the hydrogen atoms in alkyl, alkenyl, alkynyl, cycloalkyl or heterocyclic group defined in R, R¹, R², R³ and R⁴ are partially or entirely substituted by identical or different substituting groups selected from the following: halogen, cyano, hydroxyl, amino, mercapto, C₁-C₆ alkyl, C₁-C₆ alkoxy, C₁-C₆ alkylcarbonyl, C₁-C₆ alkoxycarbonyl, C₁-C₆ alkylthio, C₁-C₆ alkylamino, C₁-C₆ alkylsulfinyl, C₁-C₆ alkylsulfonyl or C₃-C₈ heterocyclic group.

13. The compound of formula (II) as the synthetic intermediate for preparing the 1-pyridyl-pyrazolyl amide compound represented by Formula (I) of claim 4, wherein a method for preparing the compound of formula (II) comprises the following steps: 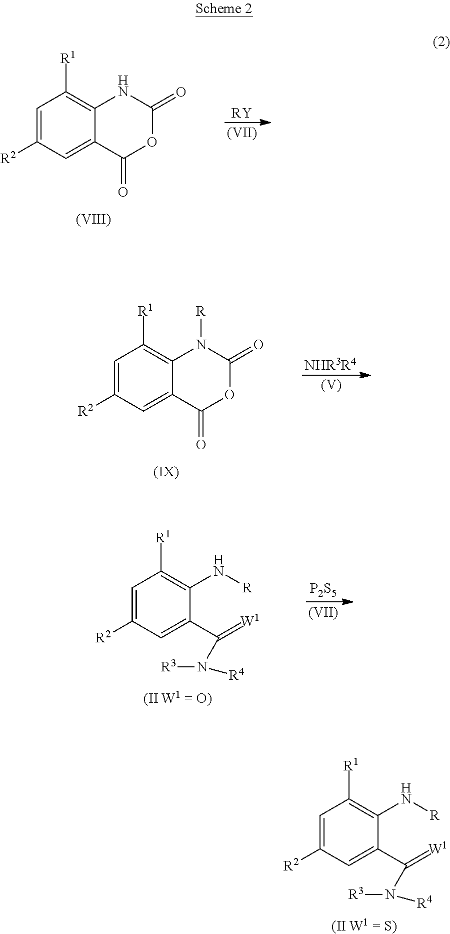 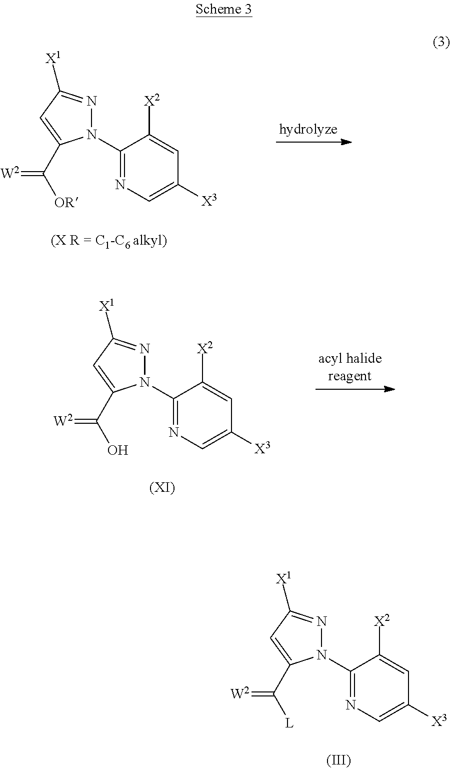 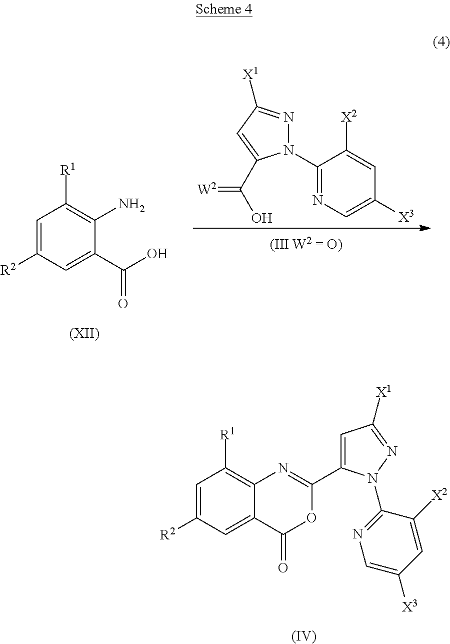 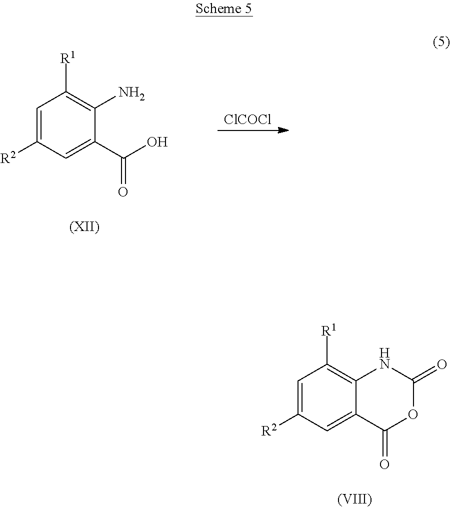
in a solvent or a mixture of any two solvents, at certain temperatures from −15° C. to the reflux temperature of the solvent used, in the presence of a base, reacting a compound of Formula (VIII) with the compound of Formula (VII) to afford the compound of Formula (IX), the solvent is selected from tetrahydrofuran, 1,4-dioxane, acetone, benzene, toluene, xylene, dichloromethane, dichloroethane, chloroform, tetrachloromethane, hexane, ethyl acetate, acetonitrile, dimethyl sulfoxide or N,N-dimethylformamide, the base is selected from triethylamine, N,N-dimethylaniline, pyridine, methylpyridine, sodium hydroxide, potassium hydroxide, sodium carbonate, potassium carbonate, sodium hydride, sodium methoxide, sodium tert-butoxide, potassium tert-butoxide or sodium bicarbonate; in a solvent or a mixture of any two solvents, at certain temperatures from −15° C. to the reflux temperature of the solvent used, in the presence of a base, reacting the compound of Formula (IX) with the compound of Formula (V) to afford a compound of Formula (II W¹=O), the solvent is selected from water, tetrahydrofuran, 1,4-dioxane, benzene, toluene, xylene, dichloromethane, dichloroethane, chloroform, tetrachloromethane, hexane, ethyl acetate, acetonitrile, dimethyl sulfoxide or N,N-dimethylformamide; in a solvent or a mixture of any two solvents, at certain temperatures from −15° C. to the reflux temperature of the solvent used, reacting the compound of Formula (II W¹=O) with P₂S₅ to afford a compound of Formula (II W¹=S), the solvent is selected from tetrahydrofuran, 1,4-dioxane, benzene, toluene, xylene, dichloromethane, dichloroethane, chloroform, tetrachloromethane, hexane, ethyl acetate, acetonitrile, dimethyl sulfoxide or N,N-dimethylformamide;
in a solvent or a mixture of any two solvents, at certain temperatures from 25° C. to the reflux temperature of the solvent used, in the presence of a base, routine hydrolysis of a compound of Formula (X) and then treating the reaction mixture with dilute acid solution such as dilute hydrochloric acid solution to afford a compound of Formula (XI), the solvent is selected from water, ethanol, methanol, tetrahydrofuran, 1,4-dioxane, benzene, toluene, xylene, dichloromethane, dichloroethane, chloroform, tetrachloromethane, hexane, ethyl acetate, acetonitrile, dimethyl sulfoxide or N,N-dimethylformamide, the base is selected from triethylamine, N,N-dimethylaniline, pyridine, methylpyridine, sodium hydroxide, potassium hydroxide, sodium carbonate, potassium carbonate, sodium hydride, sodium methoxide, sodium tert-butoxide, potassium tert-butoxide or sodium bicarbonate; in solvent-free or a solvent, at certain temperatures from 15° C. to the reflux temperature of the solvent used, reacting the compound of Formula (XI) with an acyl halide reagent to afford the compound of Formula (III), the solvent is selected from tetrahydrofuran, 1,4-dioxane, benzene, toluene, xylene, dichloromethane, dichloroethane, chloroform, tetrachloromethane, hexane, ethyl acetate or acetonitrile, the acyl halide reagent is selected from sulfoxide chloride, phosgene, phosphorus trichloride, phosphorus tribromide, phosphorus pentachloride, phosphorus pentabromide or oxalyl chloride;
in a solvent or a mixture of any two solvents, at certain temperatures from −15° C. to the reflux temperature of the solvent used, in the presence of a base, treating a mixture of a compound of Formula (XII) and a compound of Formula (III W²=O) with methylsulfonyl chloride to afford the compound of Formula (IV), the solvent is selected from tetrahydrofuran, 1,4-dioxane, acetone, toluene, xylene, acetonitrile, dichloromethane, dichloroethane or N,N-dimethylformamide, the base is pyridine or methylpyridine;
in a solvent or a mixture of any two solvents, at −15° C. to 25° C., reacting the compound of Formula (XII) with phosgene, diphosgene or triphosgene for 2 to 10 hours, and then at certain temperatures from 15° C. to the reflux temperature for 2 to 10 hours, to afford the compound of Formula (VIII), the solvent is selected from tetrahydrofuran, acetone, 1,4-dioxane, benzene, toluene, xylene, dichloromethane, dichloroethane, chloroform, tetrachloromethane, hexane, ethyl acetate or acetonitrile;
wherein R, R¹, R², R³, R⁴, X¹, X², X³, W¹ and W² are the same as defined in claim 4, L is a leaving group including chlorine or bromine.

14. An use of the 1-pyridyl pyrazole amide compound according to claim 2, wherein the compound possesses insecticidal activity comprising contacting the pests or their environment with the compound of Formula (I) at a dosage of 15 to 5000 g per hectare.

15. An use of the 1-pyridyl pyrazole amide compound according to claim 3, wherein the compound possesses insecticidal activity comprising contacting the pests or their environment with the compound of Formula (I) at a dosage of 15 to 5000 g per hectare.

16. An insecticidal composition, comprising a biologically effective amount of a compound according to claim 2 and at least one additional component selected from the group consisting of surfactants, solid diluents and liquid diluents, in which the weight percentage of the active ingredient is in the range of 0.5-99%.

17. An insecticidal composition, comprising a biologically effective amount of a compound according to claim 3 and at least one additional component selected from the group consisting of surfactants, solid diluents and liquid diluents, in which the weight percentage of the active ingredient is in the range of 0.5-99%.
